(19) United States
(12) Patent Application Publication
(10) Pub. No.: US 2026/0034130 A1
(43) Pub. Date: Feb. 5, 2026

(54) USE OF MILVEXIAN FOR TREATING OR PREVENTING ISCHEMIC STROKE

(71) Applicants: BRISTOL-MYERS SQUIBB COMPANY, Princeton, NJ (US); JANSSSEN PHARMACEUTICA NV, Beerse (BE)

(72) Inventors: Alexei PLOTNIKOV, Bridgewater, NJ (US); Christopher NESSEL, Bridgewater, NJ (US); Vidya Liyanage PERERA, Princeton, NJ (US); Danshi LI, Princeton, NJ (US); Charlotte JONES-BURTON, Princeton, NJ (US); Anja KAHL, Princeton, NJ (US); Samira J. MERALI, Princeton, NJ (US); Puneet MOHAN, Princeton, NJ (US); Yshai YAVIN, Princeton, NJ (US)

(21) Appl. No.: 19/100,198

(22) PCT Filed: Aug. 1, 2023
(86) PCT No.: PCT/US2023/029178

§ 371 Date: Jan. 31, 2025

Related U.S. Application Data
(60) Provisional application No. 63/370,031, filed on Aug. 1, 2022.
(60) Provisional application No. 63/401,361, filed on Aug. 26, 2022.
(60) Provisional application No. 63/379,848, filed on Oct. 17, 2022.
(60) Provisional application No. 63/498,945, filed on Apr. 28, 2023.

(51) Int. Cl. A61K 31/513; A61K 31/4365; A61K 31/519; A61K 31/616; A61K 31/7076; A61P 7/02; A61P 9/10

(52) U.S. Cl. CPC A61K 31/513; A61K 31/4365; A61K 31/519; A61K 31/616; A61K 31/7076; A61P 7/02; A61P 9/10

(57) ABSTRACT

A Factor XIa inhibitor having therapeutic properties useful in methods for treating or preventing ischemic stroke.

### CROSS-REFERENCE TO RELATED APPLICATIONS

This application claims the benefit of U.S. Provisional Application No. 63/370,031, filed Aug. 1, 2022; U.S. Provisional Application No. 63/401,361, filed Aug. 26, 2022; U.S. Provisional Application No. 63/379,848, filed Oct. 17, 2022; and U.S. Provisional Application No. 63/498,945, filed Apr. 28, 2023. The disclosure of each aforementioned application is incorporated by reference herein in its entirety.

### TECHNICAL FIELD

The disclosure pertains to the use of milvexian for treating or preventing ischemic stroke in patients with acute ischemic stroke or transient ischemic attack (TIA) while preserving hemostasis.

### BACKGROUND

Ischemic stroke is a devastating and common condition. Data from the Global Burden of Disease study estimated that the 2019 global prevalence of ischemic stroke was 77 million people, with the highest age-standardized incidence rates in southeast/east/central Asia, central/eastern Europe, North Africa, and the Middle East. Across Europe, stroke accounts for over 1 in 10 of all deaths each year and is the third leading cause of disability-adjusted years lost (Feigin et al., Global Burden of Stroke. Circ Res. 2017; 120:439-448). In the US, an estimated 7.6 million Americans >20 years of age self-report having had a stroke, with an additional 3.4 million US adults projected to experience a stroke by the year 2030 (Tsao et al., Heart Disease and Stroke Statistics-2022 Update: A Report From the American Heart Association. Circulation. 2022; 145 (8): e153-e639). In patients with coronary, peripheral, or cerebrovascular atherosclerosis, thromboembolic complications are the main causes of mortality worldwide.

Atherosclerosis is a chronic inflammatory disease of the arterial wall and is characterized by endothelial dysfunction leading to lipid deposition, calcium accumulation in the subendothelial space, followed by leukocyte infiltration and smooth muscle proliferation. Vessel injury, such as occurring during erosion or rupture of atherosclerotic lesions triggers blood coagulation, in attempt to maintain hemostasis (protect against bleeding). This results in thrombus formation and acute coronary syndromes (ACS). Atherothrombosis occurs as a consequence of the production of a thrombus on a rupture or erosive atherosclerotic plaque and clinically manifested as myocardial infarction (MI), peripheral artery disease (PAD), or ischemic stroke (not due to embolization from the heart).

The link between coagulation and atherothrombosis has been demonstrated in numerous animal and clinical studies (Ngo et al., Pharmacological targeting of coagulation factor XI mitigates the development of experimental atherosclerosis in low-density lipoprotein receptor-deficient mice, J Thromb Haemost. 2021; 19:1001-1017). Numerous coagulation components are present in human atherosclerotic lesions, strongly indicative of roles of coagulation activity during early plaque development. Furthermore, thrombin, tissue factor pathway inhibitor, coagulation factor (F) VIII, and activated FX (FXa) have all been shown to contribute to plaque development in mice. Components of the intrinsic pathway, such as factor XII (FXII) and more recently factor XI (FXI), have also been shown to play a role in atherogenesis in ApoE−/− mice through unclear mechanisms.

The etiology of ischemic stroke is due to either a thrombotic or embolic event that causes a decrease in blood flow to the brain. In a thrombotic event, the blood flow to the brain is obstructed within the blood vessel due to dysfunction within the vessel itself, usually secondary to atherosclerotic disease, arterial dissection, fibromuscular dysplasia, or inflammatory condition. In an embolic event, debris from elsewhere in the body blocks blood flow through the affected vessel. The etiology of stroke affects both prognosis and outcomes (Ntaios G. Embolic Stroke of Undetermined Source: JACC Review Topic of the Week. J Am Coll Cardiol. 2020 Jan. 28; 75(3):333-340; Pierik et al., Distribution of Cardioembolic Stroke: A Cohort Study. Cerebrovasc Dis. 2020; 49(1):97-104).

A key element in the pathophysiology of cerebrovascular ischemic stroke events is the activated platelet (Field et al., Antiplatelet Therapy for Secondary Prevention of Stroke, Stroke, 4^{th} Ed., Vancouver, 2015, Chapter 61, pp. 992-1013). Correspondingly, antiplatelet therapy is a cornerstone in the prevention of such events. The risk of recurrent ischemic stroke is high after an initial ischemic stroke (See Tsao et al. supra). The current standard-of-care for secondary prevention cerebrovascular events after a non-cardioembolic ischemic stroke or TIA is single antiplatelet therapy (SAPT) or dual antiplatelet therapy (DAPT), along with modification of patient level risk factors by increasing physical activity and tobacco cessation, and management of vascular risk factors including arterial hypertension, hyperlipidemia, hypertriglyceridemia, and glycemic control (Rothwell et al., Effects of aspirin on risk and severity of early recurrent stroke after transient ischemic attack and ischemic stroke: time-course analysis of randomized trials. Lancet. 2016; 388(10042):365-375. Epub 2016 May 18; Dawson et al., European Stroke Organization. European Stroke Organization expedited recommendation for the use of short-term dual antiplatelet therapy early after minor stroke and high-risk TIA. Eur Stroke J. 2021 June; 6(2): VI. Epub 2021 Jun. 18). Although the risk of recurrent ischemic stroke can be lowered by implementation of these guideline-directed approaches, the residual risk of recurrent stroke remains high, even when patients are treated with combination of aspirin and clopidogrel (See Field et al., supra) and antiplatelet therapy intensification or extended treatment duration is limited by an increased risk of bleeding.

Researchers tested in patients various strategies for achieving better efficacy, for example, using more potent or longer duration antiplatelet therapy, or combination of antiplatelet and anticoagulation therapy. However, long-term use of DAPT has not been supported by evidence from randomized studies. The MATCH study compared clopidogrel plus aspirin to clopidogrel alone as chronic therapy, initiated up to 3 months after an ischemic stroke, and did not show an efficacy benefit during 18-month treatment. The CHARISMA study compared clopidogrel plus aspirin to aspirin alone for chronic therapy, initiated any time up to 5 years after an ischemic stroke or TIA, and did not show an overall efficacy benefit during 28 months (median) of follow-up.

The risk of recurrence of stroke represents a significant and persistent unmet need for a more effective therapeutic option (Johnston et al., Ticagrelor and Aspirin or Aspirin Alone in Acute Ischemic Stroke or TIA. N Engl J Med. 2020; 383(3):207-217).

Activated coagulation factor XI (FXIa) is a key serine protease involved in the amplification of thrombin production. Increased thrombin level brings the risk of thrombosis. Inhibition of FXIa reduces thrombin generation and prevents thrombosis, while preserving normal hemostasis. While FXIa makes relatively modest contributions to hemostasis, it is involved in thrombus growth and stabilization (see FIG. 1). (Fredenburgh J C, et al. Factor XI as a target for new anticoagulants. Hamostaseologie 2021; 41(2):104-10). Epidemiologic and Bayesian randomization studies in individuals born with FXI deficiency suggest that FXI(a) inhibition is a promising strategy for secondary stroke prevention. (Gill D et al. Genetically determined FXI (factor XI) levels and risk of stroke. Stroke 2018; 49(11):2761-3; Salomon O, Steinberg D M, Koren-Morag N, et al. Reduced incidence of ischemic stroke in patients with severe factor XI deficiency. Blood 2008; 111(8):4113-7). Patients with FXI deficiency have a significantly lower risk for stroke than matched controls with normal FXI levels, whereas patients with high FXI levels have an increased risk for recurrent stroke (Rohmann J L, et al. Coagulation factor XII, XI, and VIII activity levels and secondary events after first ischemic stroke. J Thromb Haemost 2020; 18(12):3316-24).

An analysis of genetically determined FXI has shown the highest levels of FXI are associated with a heightened risk of ischemic stroke. (Gill et al., Genetically Determined FXI (Factor XI) Levels and Risk of Stroke. Stroke. 2018; 49(11):2761-2763). It has been shown that patients with congenital FXI deficiency (hemophilia C) have lower risk of stroke and venous thromboembolism (Gailani et al., Factor XI as a therapeutic target. Arterioscler Thromb Vasc Biol. 2016; 36(7):1316-1322; Peyvandi, et al; European Network of Rare Bleeding Disorders Group. Coagulation factor activity and clinical bleeding severity in rare bleeding disorders: results from the European Network of Rare Bleeding Disorders. J Thromb Haemost. 2012 April; 10(4):615-621). Studies of deep venous thrombosis prophylaxis have demonstrated a reduction in venography-defined thrombosis and favorable bleeding profiles with reduction in FXI levels or inhibition of FXIa (Buller et al. Factor XI antisense oligonucleotide for prevention of venous thrombosis. N Engl J Med 2015; 372(3):232-40; Weitz et al. Effect of osocimab in preventing venous thromboembolism among patients undergoing knee arthroplasty: the FOXTROT randomized clinical trial. JAMA 2020; 323(2):130-9; Weitz et al. Milvexian for the prevention of venous thromboembolism. N Engl J Med 2021; 385(23):2161-72). The potential for a therapeutic effect with low risk of bleeding makes FXI and FXIa promising targets for secondary stroke prevention.

The high human and financial cost of thromboembolic events underscore the need for newer and better therapeutic options for the management of thrombotic disorders. The challenge is in developing an agent that has potent antithrombotic effects but minimal bleeding risk, as it requires a very fine balancing act in modulating the hemostatic processes. Dual pathway inhibition with antiplatelet therapy and an anticoagulant is another strategy to potentially improve efficacy in patients after a recent ischemic stroke or TIA. While data from the WARSS study did not show a benefit for the use of anticoagulation with warfarin versus antiplatelet therapy with aspirin alone, the exploratory outcomes in the COMPASS trial in subjects with stable atherosclerotic vascular disease demonstrated that a dual pathway mechanism with antiplatelet therapy and an anticoagulant improves efficacy by reducing the risk of ischemic stroke with an added major bleeding liability (Eikelboom et al.; COMPASS Investigators. Rivaroxaban with or without Aspirin in Stable Cardiovascular Disease. N Engl J Med 2017; 377:1319-30). Although many treatments are available for ischemic stroke, new therapeutic options such as oral formulation of FXIa inhibitors are needed for preventing the recurrent adverse vascular events in patients who have or have had an ischemic stroke or transient ischemic attack (TIA).

Milvexian (BMS-986177/JNJ-70033093) is a direct-acting, high-affinity inhibitor of human coagulation FXIa (Dilger et al. Discovery of milvexian, a high-affinity, orally bioavailable inhibitor of factor XIa in clinical studies for antithrombotic therapy. J Med Chem 2022; 65(3):1770-85). Milvexian is a macrocyclic compound having the structure of Formula (I):

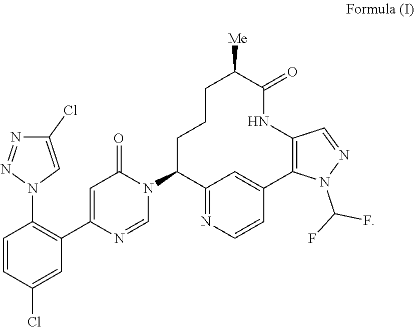 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

Milvexian is also known by its chemical name (5R,9S)-9-(4-(5-chloro-2-(4-chloro-1H-1,2,3-triazol-1-yl)phenyl)-6-oxopyrimidin-1(6H)-yl)-21-(difluoromethyl)-5-methyl-21H-3-aza-1(4,2)-pyridina-2(5,4)-pyrazolacyclonaphan-4-one.

Milvexian and a method of preparing milvexian are described in U.S. Pat. No. 9,453,018, which is hereby incorporated by reference in its entirety. Solvates, crystalline forms, and amorphous forms of milvexian are also known in the art. See, e.g., WO2021207659 and WO2022081473. An amorphous solid dispersion composition of milvexian in one or more polymers has been described in WO2020210629, which is hereby incorporated by reference in its entirety.

4,114 participants have been included and exposed to the study intervention (milvexian, placebo, or comparator) in the milvexian clinical program. Of the 4,114 participants, 3,229 received milvexian, of which 660 participants were exposed to milvexian in the Phase 1 studies and 2,569 participants were exposed to milvexian in the Phase 2 studies. Four types of serious bleeding in the milvexian clinical development program were assessed as adverse drug reactions: gastrointestinal bleeding, procedural hemorrhage, nervous system disorder bleeding (hemorrhagic transformation of ischemic stroke and subdural hematoma) and hematuria. The result of Phase II clinical trial of milvexian in patients undergoing TKR (total knee replacement) was published in 2021 (Weitz, J. I.; et al. supra).

A number of other small molecule FXIa inhibitors have been investigated in a number of studies in human for secondary prevention of ischemic stroke in patient who have or have had an ischemic stroke or transient ischemic attack (TIA). For example, in the PACIFIC-Stroke Phase II trial, safety and efficacy of three doses of asundexian (10 mg, 20 mg, or 50 mg once daily dosed for 26 to 52 weeks) when added to standard of care antiplatelet therapy was evaluated in patients after acute non-cardioembolic ischaemic stroke. Asundexian in addition to antiplatelet therapy did not reduce the composite of covert brain infarction or ischaemic stroke and did not increase the composite of major or clinically relevant non-major bleeding compared with placebo. (Shoamanesh et al., Factor XIa inhibition with asundexian after acute non-cardioembolic ischaemic stroke (PACIFIC-Stroke): an international, randomized, double-blind, placebo-controlled, phase 2b trial, Lancet, Vol. 400, Issue 10357, pp. 997-1007, DOI: https://doi.org/10.1016/S0140-6736(22)01588-4). However, the PACIFIC-AMI and PACIFIC-Stroke trials were not statistically powered to test the clinical efficacy of asundexian at a dose of 50 mg administered once daily to reduce thrombotic events such as ischemic vascular events in patients after an acute heart attack or acute non-cardioembolic ischemic stroke, in particularly by virtue of its ability to reduce the FXIa activity in the blood.

Small molecule FXIa inhibitors have been investigated in a number of studies in human. The result of Phase II clinical trial of milvexian in patients undergoing TKR (total knee replacement) was published in 2021 (Weitz, J. I.; et al, supra). In the PACIFIC-AF Phase II trial, safety of asundexian (20 mg, 50 mg QD dosed for 12 weeks) was evaluated in patients with atrial fibrillation in comparison with apixaban Piccini, J. P.; et al., Safety of the Oral Factor XIa Inhibitor Asundexian Compared with Apixaban in Patients with Atrial Fibrillation (PACIFIC-AF): a Multicentre, Randomised, Double-blind, Double-dummy, Dose-finding Phase 2 Study. Lancet, 2022, 399, 1383-1390).

Thus, vascular and thromboembolic diseases remain the leading causes of death and disability worldwide. Despite significant advances in antithrombotic treatment, anticoagulation therapy has been limited by bleeding and high thrombotic risk remains for many patients.

### SUMMARY

This disclosure provides treatment regimen comprising an oral formulation of milvexian or pharmaceutically acceptable salt or solvate thereof, a potent and highly selective FXIa inhibitor as described above, for targeted modulation of coagulation through inhibition of the contact pathway inhibiting activated factor XI (FXIa), and one or more antiplatelet therapy to treat and prevent thrombus formation and embolism, thus reducing the risks of adverse cerebrovascular events such as recurrent ischemic stroke in patients who have or have had an ischemic stroke or transient ischemic attack (TIA), preserving hemostasis while reducing thrombin generation as the result of FXIa inhibition hand-in hand with inhibition of platelet activation. The treatment regimen as described herein provides a more favorable balance between ischemia and bleeding risk as compared with antiplatelet therapy alone.

The present disclosure generally relates to methods of treating or preventing ischemic stroke in patients who have or have had an ischemic stroke or transient ischemic attack (TIA). More specifically, the present disclosure relates to the use of milvexian (or pharmaceutically acceptable salt or solvate thereof) for reducing the risk of ischemic stroke without a clinically important increase in bleeding risk compared with placebo in patients who have or have had an ischemic stroke or TIA.

In a first aspect, disclosed herein is a method comprising, in a randomized, double blind clinical trial, administering, in an experimental arm, to a patient who has or has had an ischemic stroke or TIA, an amount of milvexian (or pharmaceutically acceptable salt or solvate thereof) and DAPT effectively reducing the risk of ischemic stroke in the patient, wherein the risk in the experimental arm being numerically reduced compared, in a placebo comparator arm, with the reduction of risk in a patient who has or has had an ischemic stroke or TIA, resulting from administering to the patient a placebo and DAPT. In some embodiments of the first aspect, a pharmaceutical composition comprising 25 mg, 50 mg, 100 mg or 200 mg of milvexian (or pharmaceutically acceptable salt or solvate thereof) and a pharmaceutically acceptable carrier is administered twice daily in the experimental arm. In some embodiments of the first aspect, a pharmaceutical composition comprising 25 mg of milvexian (or pharmaceutically acceptable salt or solvate thereof) and a pharmaceutically acceptable carrier is administered once daily in the experimental arm. In some embodiments of the first aspect, a pharmaceutical composition comprising 25 mg to 200 mg of milvexian (or pharmaceutically acceptable salt or solvate thereof) and a pharmaceutically acceptable carrier is administered twice daily in the experimental arm.

In some aspects, the present disclosure provides methods of preventing ischemic stroke in a human patient who has or has had an ischemic stroke or transient ischemic attack, wherein the methods comprise administering to the human patient milvexian (or pharmaceutically acceptable salt or solvate thereof) wherein the milvexian (or pharmaceutically acceptable salt or solvate thereof) is administered in an amount of 25 mg once daily, or in an amount of 25 mg to 200 mg twice daily, and wherein the milvexian (or pharmaceutically acceptable salt or solvate thereof) is administered in addition to the standard of care.

In other aspects, the disclosure provides methods of reducing the risk of ischemic stroke in a human patient who has or has had an ischemic stroke or transient ischemic attack, the methods comprising administering to the human patient milvexian (or pharmaceutically acceptable salt or solvate thereof), wherein the milvexian (or pharmaceutically acceptable salt or solvate thereof) is administered in an amount of 25 mg once daily, or in an amount of 20 mg to 200 mg twice daily; and wherein the milvexian (or pharmaceutically acceptable salt or solvate thereof) is administered in addition to the standard of care.

In some aspects, the present disclosure provides methods of preventing ischemic stroke in a human patient who has or has had an ischemic stroke or transient ischemic attack, wherein the methods comprise administering to the human patient a regimen comprising milvexian (or pharmaceutically acceptable salt or solvate thereof), in an amount of 25 mg once daily, or in an amount of 25 mg to 200 mg twice daily, and the standard of care, wherein the regimen is effective in preventing ischemic stroke in a human patient who has or has had an ischemic stroke or transient ischemic attack, relative to the standard of care alone.

In some aspects, the disclosure provides methods of reducing the risk of ischemic stroke in a human patient who has or has had an ischemic stroke or transient ischemic attack, the methods comprising administering to the human patient a regimen comprising milvexian (or pharmaceutically acceptable salt or solvate thereof), in an amount of 25 mg once daily, or in an amount of 25 to 200 mg twice daily, and the standard of care, wherein the regimen is effective in reducing the risk of ischemic stroke in a human patient who has or has had an ischemic stroke or transient ischemic attack, relative to the standard of care alone.

In some aspects, the disclosure provides methods of treating a human patient who has or has had an ischemic stroke or transient ischemic attack, wherein the methods comprise administering to the human patient a regimen comprising milvexian (or pharmaceutically acceptable salt or solvate thereof) in an amount of 25 mg once daily, or 25 mg to 200 mg twice daily and the standard of care; wherein the regimen is effective in reducing the risk of ischemic stroke in a human patient who has or has had an ischemic stroke or transient ischemic attack and wherein the human patient does not suffer an ischemic stroke during the duration of the regimen.

In other aspects, the disclosure provides methods of treating a patient population wherein the patients in the population who have or have had an ischemic stroke or a transient ischemic attack, the methods comprising administering to the patients in the population a regimen comprising milvexian (or pharmaceutically acceptable salt or solvate thereof) in an amount of 25 mg once daily or in an amount of 25 mg to 200 mg twice daily; and the standard of care; wherein the percentage of patients in the population who experience an ischemic stroke within 90 days of beginning the regimen is less than 5%.

In other aspects, the disclosure provides milvexian for use in a method of preventing ischemic stroke in a human patient who has or has had an ischemic stroke or transient ischemic attack, the method comprising administering to the human patient a regimen comprising milvexian (or pharmaceutically acceptable salt or solvate thereof) in an amount of 25 mg once daily or in an amount of 25 mg to 200 mg twice daily; and the standard of care.

In other aspects, the disclosure provides milvexian (or pharmaceutically acceptable salt or solvate thereof) for use in preventing ischemic stroke in a human patient who has or has had an ischemic stroke or transient ischemic attack, wherein the milvexian is administered in a regimen comprising an amount of 25 mg once daily or in an amount of 25 mg to 200 mg twice daily; together with the standard of care.

In other aspects, the disclosure provides a composition comprising milvexian (or pharmaceutically acceptable salt or solvate thereof) for use in a method of preventing ischemic stroke in a human patient who has or has had an ischemic stroke or transient ischemic attack, wherein the composition is administered in a regimen comprising a unit dosage of milvexian (or pharmaceutically acceptable salt or solvate thereof) of 25 mg once daily or at a unit dosage of milvexian of 25 mg to 200 mg twice daily; together with the standard of care.

In other aspects, the disclosure provides milvexian (or pharmaceutically acceptable salt or solvate thereof) for use in a method of reducing the risk of ischemic stroke in a human patient who has or has had an ischemic stroke or transient ischemic attack, the method comprising administering to the human patient a regimen comprising milvexian (or pharmaceutically acceptable salt or solvate thereof) in an amount of 25 mg once daily or in an amount of 25 mg to 200 mg twice daily, and the standard of care; wherein the regimen is effective in reducing the risk of ischemic stroke in a human patient who has or has had an ischemic stroke or transient ischemic attack, relative to the standard of care alone.

In other aspects, the disclosure provides milvexian (or pharmaceutically acceptable salt or solvate thereof) for use in reducing the risk of ischemic stroke in a human patient who has or has had an ischemic stroke or transient ischemic attack, wherein the milvexian is administered in a regimen comprising an amount of 25 mg once daily or in an amount of 25 mg to 200 mg twice daily; together with the standard of care; wherein the regimen is effective in reducing the risk of ischemic stroke in a human patient who has or has had an ischemic stroke or transient ischemic attack, relative to the standard of care alone.

In other aspects, the disclosure provides a composition comprising milvexian (or pharmaceutically acceptable salt or solvate thereof) for use in a method of reducing the risk of ischemic stroke in a human patient who has or has had an ischemic stroke or transient ischemic attack, wherein the composition is administered in a regimen comprising a unit dosage of milvexian (or pharmaceutically acceptable salt or solvate thereof) of 25 mg once daily or at a unit dosage of milvexian of 25 mg to 200 mg twice daily; together with the standard of care, wherein the regimen is effective in reducing the risk of ischemic stroke in a human patient who has or has had an ischemic stroke or transient ischemic attack, relative to the standard of care alone.

In other aspects, the disclosure provides milvexian (or pharmaceutically acceptable salt or solvate thereof) for use in a method of treating a human patient who has or has had an ischemic stroke or transient ischemic attack, comprising administering to the human patient a regimen comprising milvexian (or pharmaceutically acceptable salt or solvate thereof) in an amount of 25 mg once daily, or 25 mg-200 mg twice daily and the standard of care; wherein the human patient does not suffer an ischemic stroke during the duration of the regimen.

In other aspects, the disclosure provides milvexian (or pharmaceutically acceptable salt or solvate thereof) for use in treating a human patient who has or has had an ischemic stroke or transient ischemic attack, wherein the milvexian is administered in a regimen comprising an amount of 25 mg once daily or in an amount of 25 mg to 200 mg twice daily, together with the standard of care, wherein the human patient does not suffer an ischemic stroke during the duration of the regimen.

In other aspects, the disclosure provides a composition comprising milvexian (or pharmaceutically acceptable salt or solvate thereof) for use in a method of treating a human patient who has or has had an ischemic stroke or transient ischemic attack, wherein the composition is administered in a regimen comprising a unit dosage of milvexian (or pharmaceutically acceptable salt or solvate thereof) of 25 mg once daily or at a unit dosage of milvexian of 25 mg to 200 mg twice daily, together with the standard of care, wherein the human patient does not suffer an ischemic stroke during the duration of the regimen.

In other aspects, the disclosure provides milvexian (or pharmaceutically acceptable salt or solvate thereof) for use in a method of treating a patient population wherein the patients have or have had an ischemic stroke or a transient ischemic attack, comprising administering to the patients in the population for a regimen comprising milvexian (or pharmaceutically acceptable salt or solvate thereof) in an amount of 25 mg once daily or in an amount of 25 mg-200 mg twice daily, and the standard of care; wherein the percentage of patients in the population who experience an ischemic stroke within 90 days of beginning the regimen is less than 5%.

In other aspects, the disclosure provides milvexian (or pharmaceutically acceptable salt or solvate thereof) for use in treating a human patient who has or has had an ischemic stroke or transient ischemic attack, wherein the milvexian is administered in a regimen comprising an amount of 25 mg once daily or in an amount of 25 mg to 200 mg twice daily, together with the standard of care, wherein the percentage of patients in the population who experience an ischemic stroke within 90 days of beginning the regimen is less than 5%.

In other aspects, the disclosure provides a composition comprising milvexian (or pharmaceutically acceptable salt or solvate thereof) for use in a method of treating a human patient who has or has had an ischemic stroke or transient ischemic attack, wherein the composition is administered in a regimen comprising a unit dosage of milvexian (or pharmaceutically acceptable salt or solvate thereof) of 25 mg once daily or at a unit dosage of milvexian of 25 mg to 200 mg twice daily, together with the standard of care, wherein the percentage of patients in the population who experience an ischemic stroke within 90 days of beginning the regimen is less than 5%.

### BRIEF DESCRIPTION OF THE DRAWINGS

FIG. 1 shows coagulation pathways. Legend: FXII, factor XII; FXIIa, activated factor XII; FXI, factor XI; FXIa, activated factor XI; FIX, factor IX; FIXa, activated factor IX; FVIIa, activated factor VII; FVII, factor VII; FX, factor X; FXa, activated factor X. See, Kakkar et al., FXI inhibition: The Holy Grail of Haemostasis-Sparing Anticoaulation, EMJ, 2021, vol. 6, pp. 12-20; and Fredenburgh et al., FXIa as a Target for New Anticoagulants, Hamostaseologie, 2021, vol. 41, pp. 104-110.

FIG. 2 shows the AXIOMATIC-SSP study design set forth in Example 1.

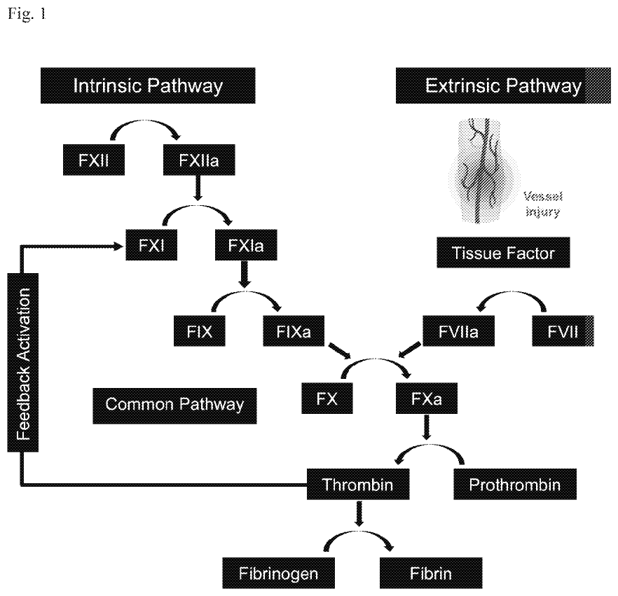

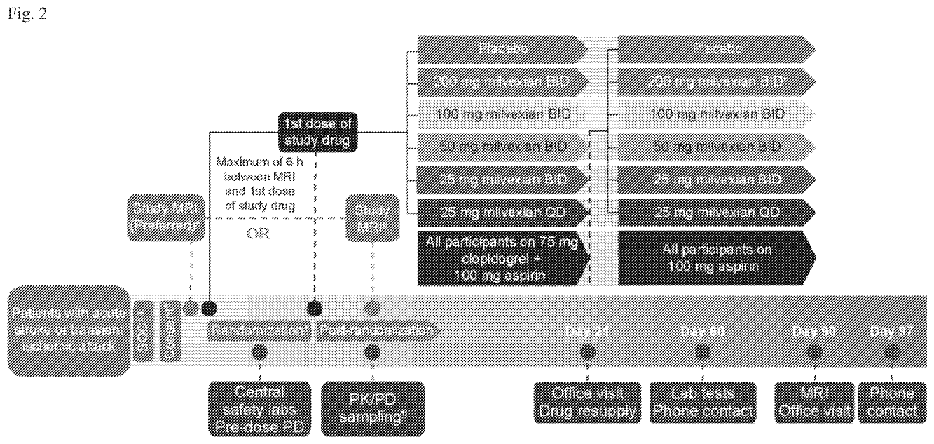

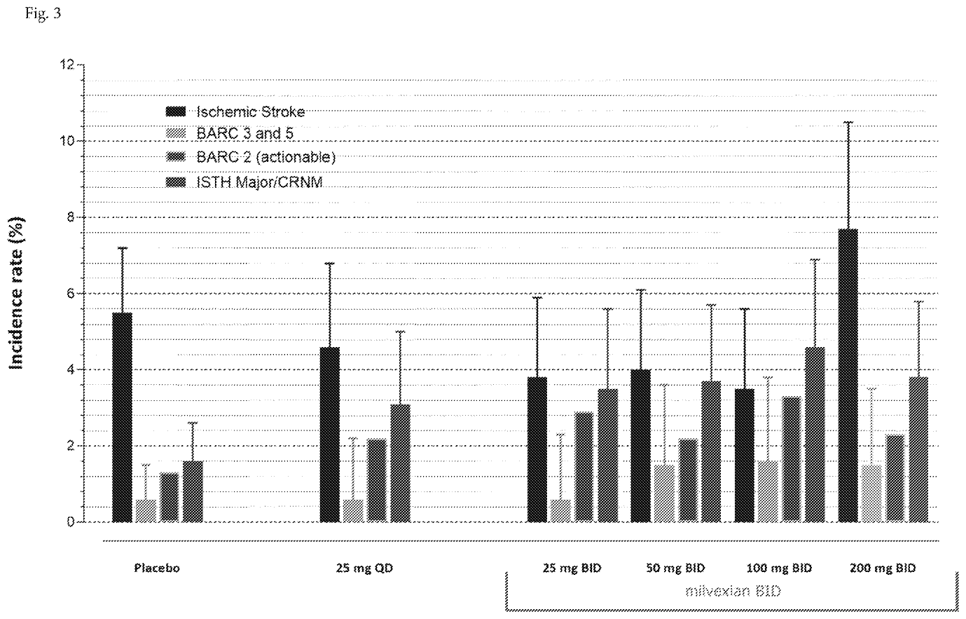

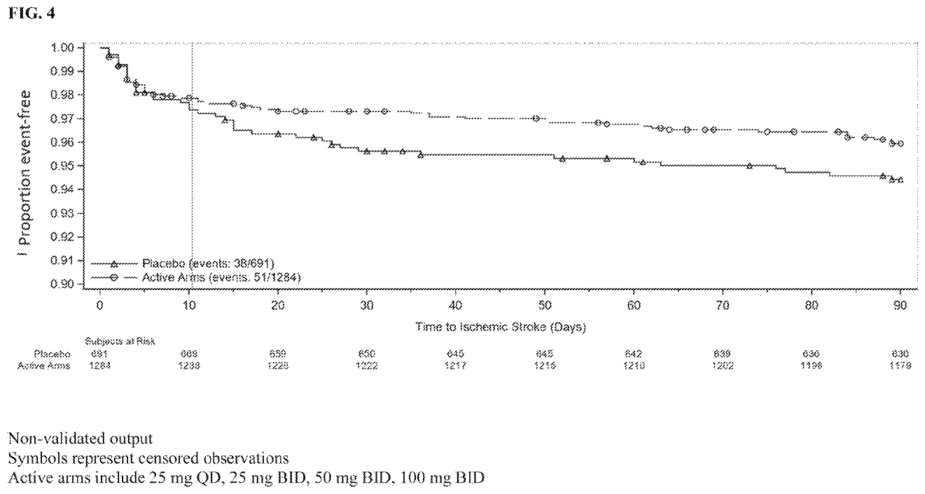

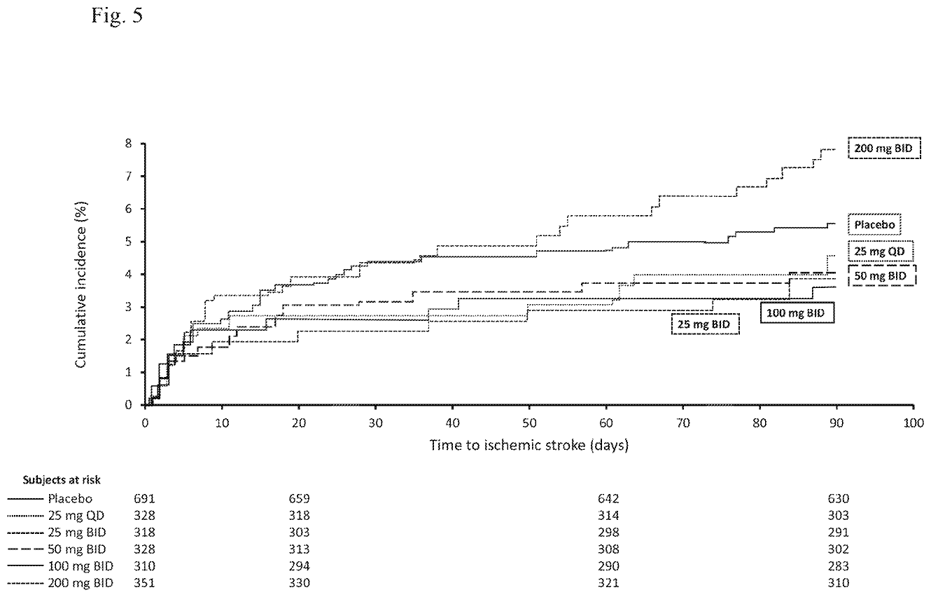

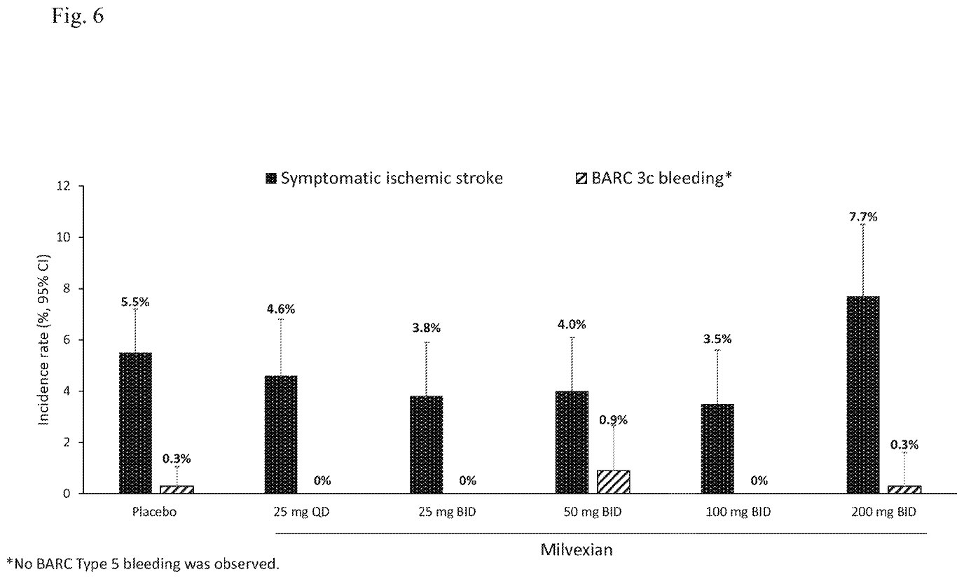

Legend: BID, twice daily; MRI, magnetic resonance imaging; QD, once daily; SOC, standard of care; PD, pharmacodynamic; PK, pharmacokinetic; LD, loading dose. *Within 48 hours of index event and prior to randomization. ^{†}300 mg clopidogrel LD+100 mg aspirin. ^{‡}600 mg clopidogrel LD permitted. ^{§} Within 72 hours of index event and up to 24 hours after randomization; clinical neuroimaging is recommended for eligibility assessment if MRI is done post-randomization (to minimize protocol deviation in case of negative imaging findings). ^{¶}Relative to the 1st or 2nd dose of study drug. ^{∥}The 200-mg BID cohort will be added after 450 participants from the lower doses completed the day 21 visit.

FIG. 3 shows the incidence rates of ischemic stroke and major bleeding by group in the clinical study described in Example 1. FIG. 3 depicts that milvexian numerically reduced the incidence rate of clinical ischemic stroke in the patients who have had an ischemic stroke or high risk TIA of the treatment arm at all doses except 200 mg BID. Doses from 25-100 mg BID showed similar relative risk reduction (RRR) of ischemic stroke at approximately 30% as compared with placebo, Similar trend observed within 21 days (the intended treatment period with DAPT). FIG. 3 also depicts that the incidence rates of major bleeding (BARC Type 3 and 5) for milvexian at 25 mg QD and BID doses was the same as placebo (0.6%).

FIG. 4 depicts the Kaplan-Meier plot of time to ischemic stroke and undetermined stroke in all randomized subjects in placebo, 25 mg QD, 25 mg BID, 50 mg BID, and 100 mg BID dose arms. The Kaplan-Meier plot depicts the time from randomization to the first occurrence of any event of ischemic stroke for pooled doses of milvexian. The vertical line indicates the time point (about 10 days, early apparent benefit) for apparent onset of the clinical benefit of milvexian treatment vs. the participants taken placebo. The reduction in the occurrence rate of ischemic stroke events took place relatively early after treatment initiation. The benefits imparted by milvexian as compared with placebo was maintained throughout the treatment period (e.g., 90 days).

FIG. 5 depicts a Kaplan-Meier plot of time course of symptomatic ischemic stroke in the intent-to-treat population in the placebo, 25 mg QD, 25 mg BID, 50 mg BID, 100 mg BID, and 200 mg BID dose arms.

FIG. 6 depicts the incidence rates (%, 95% CI) for symptomatic ischemic stroke and BARC Type 3c bleeding in the placebo, 25 mg QD, 25 mg BID, 50 mg BID, 100 mg BID, and 200 mg BID dose arms.

FIG. 7 depicts the incidence rates (%, 95% CI) for the composite of ischemic stroke (IS) and transient ischemic attack (TIA) in the placebo, 25 mg QD, 25 mg BID, 50 mg BID, 100 mg BID, and 200 mg BID dose arms. These data show a lower rate of recurrent symptomatic ischemic strokes and TIA with milvexian at all doses except 200 mg BID. Doses from 25 mg QD to 100 mg BID numerically reduced symptomatic ischemic stroke and TIA by 27% to 40% vs placebo (Wald 95% CI within group. 95% CIs for RR are constructed using Wald Confidence Limits. Clinical events are included up to Day 90.).

FIG. 8A shows the Day 1 milvexian plasma concentration as a function of time after BID administration of a film-coated direct compression tablet (2×100 mg) of the disclosure compared to the Day 1 milvexian plasma concentration as a function of time after BID administration of a milvexian-containing capsule (2×100 mg). See Example 2.

FIG. 8B shows the Day 5 milvexian plasma concentration as a function of time after BID administration of a film-coated direct compression tablet (2×100 mg) of the disclosure compared to the Day 5 milvexian plasma concentration as a function of time after BID administration of a milvexian-containing capsule (2×100 mg). See Example 2.

FIG. 8C shows the Day 1 milvexian plasma concentration as a function of time after BID administration of a film-coated direct compression tablet (1×25 mg) of the disclosure compared to the Day 1 milvexian plasma concentration as a function of time after BID administration of a milvexian-containing capsule (1×25 mg). See Example 2.

FIG. 8D shows the Day 5 milvexian plasma concentration as a function of time after BID administration of a film-coated direct compression tablet (1×25 mg) of the disclosure compared to the Day 5 milvexian plasma concentration as a function of time after BID administration of a milvexian-containing capsule (1×25 mg). See Example 2.

FIG. 9 shows the benefit-risk profile of milvexian in the Phase II AXIOMATIC-SSP study. See Example 1.

### DETAILED DESCRIPTION OF ILLUSTRATIVE EMBODIMENTS

As used herein, “preventing” refers to reducing the risk of occurrence. In some embodiments, preventing encompasses eliminating the risk of occurrence (i.e., reducing the risk of occurrence to zero). As such, “prevention” covers the preventive treatment aimed at reducing the probability of the occurrence of a clinical disease-state. In some embodiments, for any of the method described herein, “preventing” covers the reduction of recurrence of ischemic stroke or TIA.

In some embodiments, “preventing” is synonymous with “reducing the risk” or “reducing the incidence rate” of an adverse cerebrovascular event (e.g., non-cardioembolic ischemic stroke) occurring. Reducing the risk or reducing the incidence rate means that there is numerical and/or a statistically-significant reduction or lowering in occurrence of the adverse cerebrovascular event by at least 1% or greater. Preferably, this reduction is by 2% or greater, 3% or greater, 4% or greater, 5% or greater, 6% or greater, 7% or greater, 10% or greater, 20% or greater, 26% or greater, 34% or greater, 50% or greater, 64% or greater and 74% or greater. These reductions include confidence intervals equals or greater than 50%, equals or greater than 75%, equals or greater than 80%, equals or greater than 90%, equals or greater than 95%, equals or greater than 98% and equals or greater than 99%. Confidence intervals of equals or greater than 95% are preferred.

Within the scope of this disclosure, “prophylaxis” is the protective treatment of a disease state to reduce and/or minimize the risk and/or reduction in the risk of recurrence of a disease state by administering to a patient a therapeutically effective amount of milvexian or a pharmaceutically acceptable salt, or a solvate thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, the term “treating” refers to ameliorating the signs or symptoms of a disease or condition in a patient and/or preventing a disease or condition in a patient. As used herein, unless otherwise noted, the terms “treating”, “treatment” and the like, shall include the management and care of a patient for the purpose of combating a disease, condition, or disorder and includes the administration of milvexian to prevent the onset of the symptoms or complications, alleviate the symptoms or complications, or eliminate the disease, condition, or disorder. As such, “treating” or “treatment” cover the treatment of a disease-state in a human, and include: (a) inhibiting the disease-state, i.e., arresting its development; and/or (b) relieving the disease-state, i.e., causing regression of the disease state.

As used herein, the term “patient population” refers to a specific group of individual patients suffering from an indication, such as, for example, the group of patients in a particular treatment arm of a clinical trial.

As used herein, a “risk factor” is a demographic factor that influences the underlying risk of an event independently of any drug treatment.

As used herein, the term “cerebrovascular disorders” or “cerebrovascular disease” refers to the neurologic problems resulting from the disruption of the blood flow to the brain that leads to damage or death of brain cells from the lack of oxygen. In some embodiments, the cerebrovascular disorders include transient ischemic attack. In some embodiments, the cerebrovascular disorders include stroke. How a stroke or transient ischemic attack affects the body depends on precisely where in the brain the blood supply was cut off.

As used herein, the term “stroke” (also called a cerebrovascular accident) refers to an acute episode of neurologic dysfunction that caused the death of brain tissue (cerebral infarction) resulting from lack of blood flow and insufficient oxygen to the brain. A stroke can either be ischemic or hemorrhagic. In an ischemic stroke, the blood supply to part of the brain is cut off because a blood clot has blocked a blood vessel due to either a thrombus formed locally in an abnormal artery (e.g., atherosclerosis) or an embolus that formed upstream and traveled through the bloodstream. In a hemorrhagic stroke, a blood vessel bursts, preventing normal blood flow and allowing blood to leak into an area of the brain and destroy it. Most strokes begin suddenly, develop rapidly and cause brain damage within minutes (complete stroke). Less commonly, strokes may continue to worsen for several hours to days as a steadily enlarging area of brain tissue dies (stroke in evolution). Ischemic stroke may be lacunar or non-lacunar in nature. Ischemic stroke may be cardioembolic ischemic stroke if the ischemic stroke is attributable to arterial occlusion from embolus that presumably arose from the blood clots formed in the heart or on one of its valves. In some embodiments, the ischemic stroke is non-cardioembolic ischemic stroke. In some embodiments, the term “ischemic stroke” and “symptomatic ischemic stroke” may be used interchangeably.

In some embodiments, ischemic stroke refers to a neurological deficit attributable to a non-lacunar, acute brain infarction detected by neuroimaging (CT or MRI) and relevant to the clinical symptoms.

In some embodiments, the ischemic stroke is further characterized by a National Institutes of Health Stroke Score (NIHSS)≤7.

In other embodiments, the ischemic stroke is further characterized by a National Institutes of Health Stroke Score (NIHSS) of 8-15.

In other embodiments, the ischemic stroke is further characterized by a National Institutes of Health Stroke Score (NIHSS)≤15.

In other embodiments, an ischemic stroke is further characterized by evidence of relevant intracranial or cervical arterial atherosclerotic plaque, ulceration or thrombus in a feeding artery documented by imaging (either Doppler ultrasound or CTA or MRA or catheter angiography).

In still other embodiments, an ischemic stroke is further characterized by a Modified Rankin Score. For example, in some embodiments an ischemic stroke is further characterized by a Modified Rankin Score (mRS) of ≤3, of ≤4, of ≤5, or of ≤6.

As used herein, the term “transient ischemic attack (TIA)” (also known as ministroke) refers to a temporary period of focal neurological symptoms similar to those in a stroke. A TIA doesn't cause permanent damage. A TIA is caused by a temporary decrease in blood supply to part of the brain, which may last for a few minutes. Like an ischemic stroke, a TIA occurs when a clot or debris reduces or blocks blood flow to part of the nervous system. (e.g., high-risk TIA).

In some embodiments, TIA may be “high-risk transient ischemic attack” that is an acute onset neurological deficit attributable to focal ischemia of the brain by history or examination, with complete resolution of the deficit and no brain infarction on neuroimaging (CT or MRI)

In some embodiments, a high-risk transient ischemic attack is further characterized by an ABCD2 SCORE ≥6 or is characterized by motor symptoms. See Rothwell P M, et al. A simple score (ABCD) to identify individuals at high early risk of stroke after transient ischaemic attack. Lancet. 2005 Jul. 2-8; 366(9479):29-36.

In other embodiments, a high-risk transient ischemic attack is further characterized evidence of relevant intracranial or cervical arterial atherosclerotic plaque, ulceration or thrombus in a feeding artery documented by imaging (either Doppler ultrasound or CTA or MRA or catheter angiography).

As used herein, “pharmaceutically acceptable salt” refers to derivatives wherein a compound is modified by making an acid or a basic salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of milvexian can be synthesized using conventional chemical methods. Generally, such salts can be prepared by reacting t milvexian with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.

As used herein, the term “standard of care” refers to a treatment process that is generally accepted by medical experts as a proper treatment for a certain type of disease (e.g., stroke) and that is widely used by healthcare professionals. The current standard-of-care for secondary prevention of recurrent cerebrovascular events after a noncardioembolic ischemic stroke or TIA is SAPT or DAPT, along with modification of patient level risk factors by increasing physical activity and tobacco cessation, and management of vascular risk factors including arterial hypertension, hyperlipidemia, hypertriglyceridemia, and glycemic control (Antithrombotic Trialists' Collaboration 2002, Antithrombotic Trialists' Collaboration 2009, Rothwell 2016, Kleindorfer 2021, Dawson 2021, Fonseca 2021). Current AHA/ASA guidelines recommend early initiation (ideally within 12-24 hours of symptom onset and at least within 7 days of onset) of short term (21 to 90 days) DAPT with ASA plus clopidogrel, followed by antiplatelet monotherapy in patients with recent minor (NIHSS score ≤3) non-cardioembolic ischemic stroke or high-risk TIA (ABCD2 score ≥4) (Kleindorfer 2021). For patients with recent (<24 hours) minor to moderate stroke (NIHSS score ≤5), high-risk TIA (ABCD2 score ≥6), or symptomatic intracranial or extracranial stenoses as specified in the guideline, DAPT with ticagrelor plus ASA for 30 days may be considered (Kleindorfer 2021). ESO guidelines recommend DAPT with ASA and clopidogrel for 21 days followed by SAPT in minor noncardioembolic ischemic stroke (NIHSS score ≤3) or high-risk TIAs (ABCD2 score ≥4), or 30 days of DAPT with ASA plus ticagrelor followed by SAPT for patients with mild to moderate noncardioembolic ischemic stroke (NIHSS score of ≤5) or high-risk TIA (ABCD2 score of ≥6 or other high-risk features) (Dawson 2021). In some embodiments, stroke standard of care is one that a clinician generally follows for treatment of ischemic stroke in a human patient who has had a recent (within 0-48 hours) ischemic stroke or transient ischemic attack. See, e.g., AHA/ASA Guidelines, i.e., 2021 Guideline for the Prevention of Stroke in Patients With Stroke and Transient Ischemic Attack: A Guideline From the American Heart Association/American Stroke Association, Stroke, 2021; 52:e364-e467. DOI: 10.1161/STR.0000000000000375.

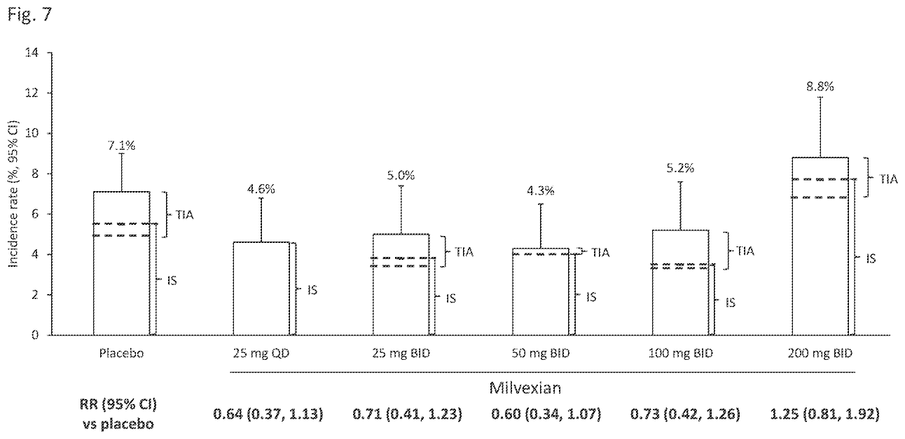

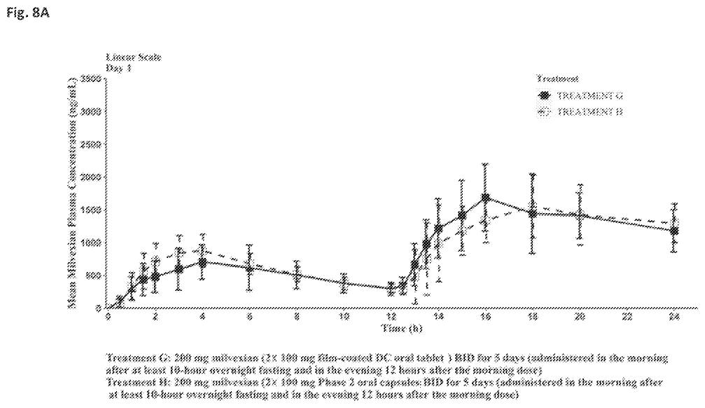

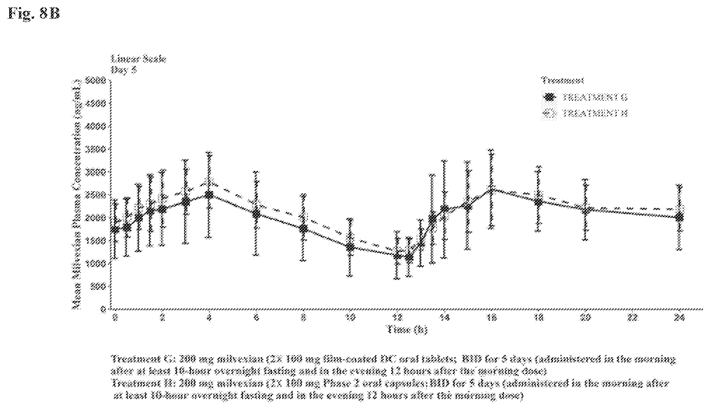

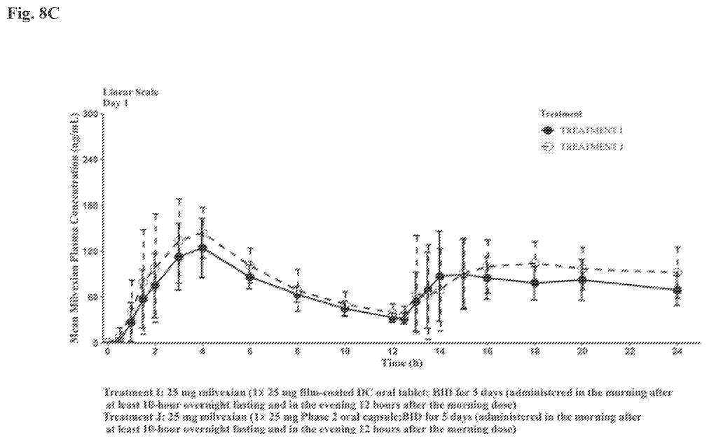

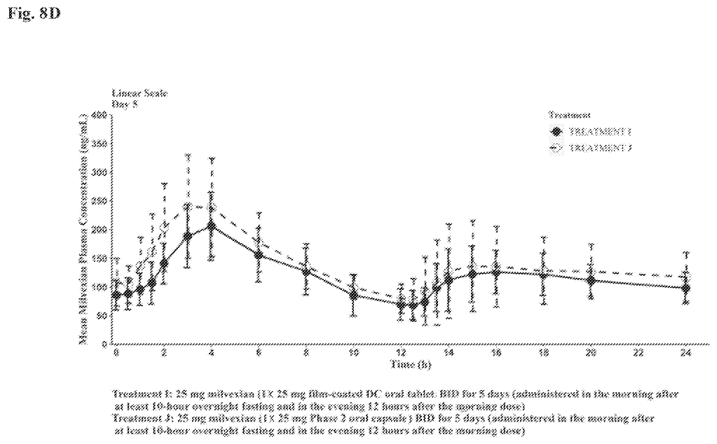

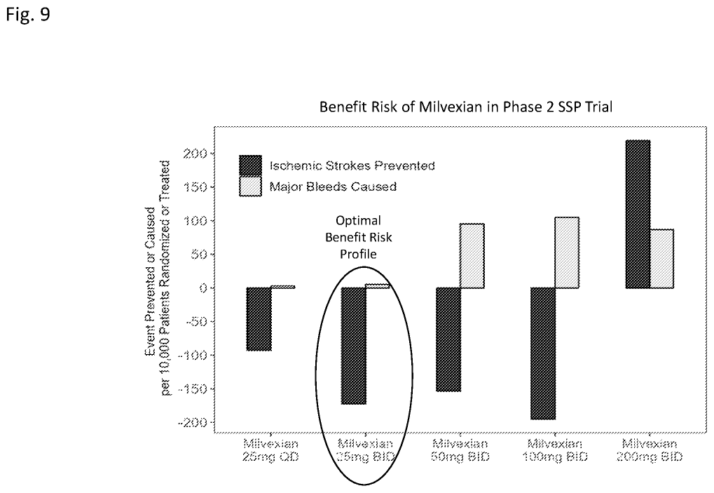

As used herein, the term “placebo dosing” or “placebo” refers to standard of care as defined above. In some embodiments, “placebo dosing” or “placebo” refers to a antiplatelet dose regimen comprising dual antiplatelet therapy (DAPT) or single antiplatelet therapy (SAPT). In some embodiments, “placebo dosing” or “placebo” refers to DAPT comprising a daily dose of 75 mg clopidogrel and 100 mg aspirin (see FIG. 2). In some aspects, the placebo is referred to as the “control.”

As used herein “safe” means that the regimen provides a net clinical benefit such that the risk reduction for ischemic stroke (or cardiovascular death, or myocardial infarction) in a human outweighs the increased risk of an adverse event, for example, serious bleeding, as compared to antiplatelet therapy alone, as determined by a regulatory agency.

As used herein “effective” means that the regimen provides risk reduction and treatment for ischemic stroke (or cardiovascular death, or myocardial infarction) as compared to antiplatelet therapy alone.

As used herein, the term “clinically proven” means that the safety and/or efficacy of the regimen is demonstrated by clinical investigation such as, for example, at least one clinical trial.

Absolute risk reduction (ARR) is the percentage of patients in the control group (i.e., the group not receiving the regimen) who have a bad outcome minus the percentage of patients in the treatment group (i.e., the group receiving the regimen) who have a bad outcome. For example, if the incidence rate in the control group is 20% (i.e., if 20% of the patients in the control group experience a bad outcome), and the incidence rate in the treatment group is 12% (i.e. 12% of the patients in experience a bad outcome), then the absolute risk reduction (ARR) is 8% (i.e., 20%-12%).

“Relative Risk” (RR) is the proportion of bad outcomes (e.g., suffering an ischemic stroke) in the treatment group (i.e., the group receiving the regimen) divided by the proportion of bad outcomes in the control group (i.e., the group not receiving the regimen). For example, if 20% of the patients in the control group experience a bad outcome, and 12% of the patients in the treatment group experience a bad outcome, then the RR=0.12/0.20=0.6. Thus, an RR of less than 1 indicates a reduction in risk.

As used herein, the term “Relative Risk Reduction” (RRR) refers to how much the regimen reduced the risk of bad outcomes relative to the control group who did not receive the regimen. For example, if 20% of the patients in the control group experience a bad outcome, and 12% of the patients in the treatment group experience a bad outcome, then the RRR=(1−RR)*100%=[1−(0.12/0.20)]*100%=0.4*100%=40%. Thus, an RRR of greater than 0% indicates a reduction in risk.

As used herein, the term “dose-dependent manner” means that the risk of occurrence is inversely proportional to the quantity of milvexian administered.

As used herein, the term “duration of the regimen” refers to the time period during which the patient is administered the regimen.

As used herein, the “Bleeding Academic Research Consortium (BARC) Type 3 criteria” are: a. Overt bleeding plus hemoglobin drop of 3 to <5 g/dL (provided hemoglobin drop is related to bleed); transfusion with overt bleeding; b. Overt bleeding plus hemoglobin drop ≥5 g/dL (provided hemoglobin drop is related to bleed); cardiac tamponade; bleeding requiring surgical intervention for control; bleeding requiring IV vasoactive agents; or c. Intracranial hemorrhage confirmed by autopsy, imaging, or lumbar puncture; intraocular bleed compromising vision. See, e.g., Roxana Mehran, et al. Standardized Bleeding Definitions for Cardiovascular Clinical Trials, Circulation. 2011; 123:2736-2747.

As used herein, the “Bleeding Academic Research Consortium (BARC) Type 5 criteria” are: a. Probable fatal bleeding; or b. Definite fatal bleeding (overt or autopsy or imaging confirmation). See, e.g., Roxana Mehran, et al. Standardized Bleeding Definitions for Cardiovascular Clinical Trials, Circulation. 2011; 123:2736-2747.

As used herein, the “Bleeding Academic Research Consortium (BARC) Type 2 criteria” are: any clinically overt sign of hemorrhage that “is actionable” and requires diagnostic studies, hospitalization, or treatment by a health care professional. See, e.g., Roxana Mehran, et al. Standardized Bleeding Definitions for Cardiovascular Clinical Trials, Circulation. 2011; 123:2736-2747.

As used herein, the “ISTH criteria (major bleeding or clinically-relevant non-major bleeding (CRNM)) criteria” are as follows: ISTH major bleeding in non-surgical patients is defined as having a symptomatic presentation and:
i. Fatal bleeding, and/or
ii. Bleeding in a critical area or organ, such as intracranial, intraspinal, intraocular, retroperitoneal, intra-articular or pericardial, or intramuscular with compartment syndrome, and/or
iii. Bleeding causing a fall in hemoglobin level of 20 g L⁻¹ (1.24 mmol L⁻¹) or more, or leading to transfusion of two or more units of whole blood or red cell

ISTH CRNM bleeding is any sign or symptom of hemorrhage (e.g., more bleeding than would be expected for a clinical circumstance, including bleeding found by imaging alone) that does not fit the criteria for the ISTH definition of major bleeding but does meet at least one of the following criteria:
i. requiring medical intervention by a healthcare professional
ii. leading to hospitalization or increased level of care
iii. prompting a face to face (i.e., not just a telephone or electronic communication) evaluation

It will be understood that the use of terms of approximation, such as “approximately” or “about,” when referring to, for example, amounts, durations of time, extents of effects, and the like, may be a number that is within 5% (greater or less than), or within 7.5% (greater or less than), or within 10% (greater or less than), or within 12.5% (greater or less than), or within 15% (greater or less than), or within 17.5% (greater or less than), or within 20% (greater or less than) of the specified number.

It will be understood that the use of the term “comparable”, when referring to, for example, amounts, durations of time, extents of effects, and the like, may be a number that is within 0% (greater or less than), or within 0.5% (greater or less than); or within 1.0% (greater or less than); or within 2.0% (greater or less than); or within 3.0% (greater or less than); or within 4.0% (greater or less than); or within 5.0% (greater or less than) of the specific number. In some embodiments, “comparable” refers to an extent of effects measured by a numeric value that is within 0% (greater or less than) of the specific number, for example, the incidence rate (%) of major bleeding (BARC Type 3 and 5) for milvexian 25 mg QD and BID doses was the same as placebo (0.6%). In some embodiments, “comparable” means statistically not different. Statistics can be interpreted at a 2-sided significance level of 0.05, and all confidence intervals will be calculated at a 2-sided confidence level of 95%.

As used herein, the term “intent-to-treat population” (ITT population) refers to all Randomized Participants who (i) were randomized to a treatment, regardless of whether they received study drug or not. For the primary efficacy endpoint analysis, the population includes all Randomized Participants who: have a primary endpoint event (a new ischemic stroke during the study period or new covert brain infarction detected by MRI on day 90); or (ii) have an evaluable MRI image on day 90. All other efficacy analyses are performed on the ITT population during Double-Blind Treatment Period. This population will be analyzed according to the treatment assigned at randomization. All treated participants include all participants who receives at least one dose of study medication. This will be the primary population used in safety analyses and will be analyzed according to the treatment assigned at randomization.

As used herein, the terms “patient who has or has had an ischemic stroke or transient ischemic attack” or “patients who have or have had an ischemic stroke or transient ischemic attack” refers to patients who have an ischemic stroke or transient ischemic attack prior to beginning to receive the treatment regimen comprising milvexian (or pharmaceutically acceptable salt or solvate thereof).

As used herein, the term “activated partial thromboplastin time (aPTT)” refers to a measure of the intrinsic and final common pathways of the coagulation cascade. It represents the time, in seconds, for plasma to clot after addition of phospholipid, an intrinsic pathway activator, and calcium. The name ‘Activated Partial Thromboplastin Time’ comes from the original form of the test in which only the phospholipid concentration of the test was controlled (as opposed to the phospholipid and the surface activator concentrations) and the name ‘partial thromboplastin’ was applied at the time to phospholipid preparations that accelerated clotting but did not correct the prolonged clotting times of hemophilic plasma. The term ‘partial’ means phospholipid is present but no tissue factor. The normal and reference ranges vary depending on reagent and instrument combinations, particularly with the phospholipid composition.

In some embodiments, aPTT is measured as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT).

As used herein, “Factor XI Clotting Activity” is determined utilizing an aPTT-based 1-stage clotting time assay. Serial dilutions of normal pooled plasma are mixed with FXI-depleted plasma and the clotting times are measured according to standard aPTT protocol, to establish a reference range. Subject test plasma is treated in the same way and compared with the reference plasma.

In some embodiments, the Factor XI Clotting Activity is measured as follows: Factor XI (FXI) activity is measured using a modification of the activated partial thromboplastin time (aPTT) using Actin FS (Siemens Healthcare) on the Siemens BCS®XP analyzer. A 6-point calibration curve (˜5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS®XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FM and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS®XP in saline.

In some aspects, the methods of the disclosure are directed to preventing ischemic stroke in adult patients after an acute ischemic stroke or high risk transient ischemic attack. In other aspects, the methods of the disclosure are directed to reducing the risk of ischemic stroke in adult patients after an acute ischemic stroke or high risk transient ischemic attack. As used here, “after” is synonymous with “has or has had” and “have or have had.”

In other aspects, the methods of the disclosure are directed to preventing ischemic stroke in a human patient who has or has had an ischemic stroke or transient ischemic attack.

In some aspects, the methods of the disclosure are directed to reducing the risk of ischemic stroke in a human patient who has or has had an ischemic stroke or transient ischemic attack.

In some aspects, the disclosure provides methods of treating a human patient who has or has had an ischemic stroke or transient ischemic attack.

In other aspects, the disclosure provides methods of treating a patient population wherein the patients have or have had an ischemic stroke or a transient ischemic attack.

The methods of the disclosure are performed on human patients. In some embodiments, the human patient is a female. In other embodiments, the human patient is a male.

In some embodiments, the human patient is at least 40 years old.

In other embodiments, the human patient is at least 50 years old.

In other embodiments, the human patient is at least 60 years old.

In other embodiments, the human patient is at least 70 years old.

In some aspects of the disclosed methods, the patient is administered a regimen comprising milvexian, or a pharmaceutically acceptable salt or solvate thereof.

In some embodiments, the regimen comprises milvexian.

In some embodiments, the milvexian in the regimen comprises a solvate of milvexian.

In other embodiments, the regimen comprises a pharmaceutically acceptable salt of milvexian.

In embodiments of the disclosed methods in which the regimen comprises a solvate or a pharmaceutically acceptable salt of milvexian, then the amount specified is on a milvexian basis. That is, an amount of the solvate or the pharmaceutically acceptable salt in the regimen contains the specified amount of milvexian. For example, a regimen comprising 50 mg of milvexian (or pharmaceutically acceptable salt or solvate thereof) refers to a regimen comprising either 50 mg of milvexian, or an amount of a pharmaceutically acceptable salt or solvate of milvexian equivalent to 50 mg of milvexian.

In some aspects, the methods of the disclosure comprise administering to the human patient (or the patients in the patent population) a regimen comprising milvexian (or pharmaceutically acceptable salt or solvate thereof) in an amount of 12.5 mg to 200 mg twice daily; and the standard of care.

In some embodiments, the methods of the disclosure comprise administering to the human patient (or the patients in the patent population) a regimen comprising milvexian (or pharmaceutically acceptable salt or solvate thereof) in an amount of 25 mg once daily or in an amount of 25 to 200 mg twice daily; and the standard of care.

In some embodiments, the regimen comprises milvexian (or pharmaceutically acceptable salt or solvate thereof) in an amount of 12.5 mg twice daily and the standard of care.

In some embodiments, the regimen comprises milvexian (or pharmaceutically acceptable salt or solvate thereof) in an amount of 25 mg once daily and the standard of care.

In some embodiments, the regimen comprises milvexian (or pharmaceutically acceptable salt or solvate thereof) in an amount of 25 mg twice daily and standard of care, wherein the standard of care allows a loading dose of antiplatelet agents.

In other embodiments, the regimen comprises milvexian (or pharmaceutically acceptable salt or solvate thereof) in an amount of 12.5 mg to 200 mg twice daily and the standard of care. Thus, in some embodiments, the regimen comprises milvexian in an amount of, for example, one of 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 21 mg, 22 mg, 23 mg, 24 mg, 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg, twice daily, and the standard of care.

In other embodiments, the regimen comprises milvexian (or pharmaceutically acceptable salt or solvate thereof) in an amount of 25 mg to 200 mg twice daily and the standard of care. Thus, in some embodiments, the regimen comprises milvexian in an amount of, for example, one of 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg, twice daily, and the standard of care.

In other embodiments, the regimen comprises milvexian (or pharmaceutically acceptable salt or solvate thereof) in an amount of 12.5 mg to less than 200 mg twice daily and the standard of care. Thus, in some embodiments, the regimen comprises milvexian in an amount of, for example, one of 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 21 mg, 22 mg, 23 mg, 24 mg, 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, or 190 mg, twice daily, and the standard of care.

In other embodiments, the regimen comprises milvexian (or pharmaceutically acceptable salt or solvate thereof) in an amount of 25 mg to less than 200 mg twice daily and the standard of care. Thus, in some embodiments, the regimen comprises milvexian in an amount of, for example, one of 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, or 190 mg, twice daily, and the standard of care.

In some embodiments of the disclosed methods, the milvexian is administered in an amount of 25 mg once daily.

In some embodiments of the disclosed methods, the milvexian is administered in an amount of 12.5 mg twice daily.

In some embodiments of the disclosed methods, the milvexian is administered in an amount of 25 mg twice daily.

In some embodiments of the disclosed methods, the milvexian is administered in an amount of 50 mg twice daily.

In some embodiments of the disclosed methods, the milvexian is administered in an amount of 100 mg twice daily.

In some embodiments of the disclosed methods, the milvexian is administered in an amount of 200 mg twice daily.

In some embodiments of the disclosed methods, the milvexian is administered in an amount of less than 200 mg twice daily.

In some aspects, the disclosure is directed to methods of preventing, or reducing the risk of, ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT).

In other aspects, the disclosure is directed to milvexian for use in preventing, or reducing the risk of, ischemic stroke in a human patient who has had a previous ischemic stroke or transient ischemic attack, which is administered in an amount of 25 mg twice daily, with a single antiplatelet therapy (SAPT) or dual antiplatelet therapy (DAPT).

In some aspects, the regimen used in the disclosed methods comprises the standard of care.

In some embodiments, the standard of care comprises antiplatelet therapy.

In some embodiments, for any of the methods disclosed herein, individualized standard of care, antiplatelet therapy (DAPT or SAPT) and the specific P2Y12 inhibitor (clopidogrel, ticagrelor, ticlopidine, cangrelor, or prasugrel), is administered to the patient.

In some embodiments, for any of the methods disclosed herein, the standard of care comprises clopidogrel and aspirin for 21 days then clopidogrel alone or aspirin alone from day 22 through day 90.

In some embodiments, for any of the methods disclosed herein, the standard of care comprises clopidogrel and aspirin for 90 days or aspirin alone for 90 days.

In some embodiments, for any of the methods disclosed herein, the standard of care comprises clopidogrel and aspirin for 90 days.

In some embodiments, for any of the methods disclosed herein, the standard of care comprises ticagrelor alone for 90 days, or aspirin alone for 90 days.

In some embodiments, for any of the methods disclosed herein, the standard of care comprises ticagrelor alone for 90 days.

In some embodiments, for any of the methods disclosed herein, the standard of care comprises ticagrelor and aspirin for 30 days.

In some embodiments, for any of the methods disclosed herein, the antiplatelet therapy comprises a selective inhibitor of phosphodiesterase type 3 (PDE3) that inhibits blood clot formation by increasing cAMP. In some embodiments, the selective PDE3 inhibitor is cilostazol.

In some embodiments, for any of the methods disclosed herein, the antiplatelet therapy comprises a dual inhibitor of nucleoside transport and PDE3 that inhibits blood clot formation. In some embodiments, the dual inhibitor of nucleoside transport and PDE3 is dypiradamole.

In some embodiments, for any of the methods disclosed herein, the antiplatelet therapy comprises cilostazol or dypiradamole alone. In some embodiments, for any of the methods disclosed herein, the antiplatelet therapy comprises cilostazol and a P2Y12 inhibitor. In some embodiments, for any of the methods disclosed herein, the antiplatelet therapy comprises cilostazol and clopidogrel alone.

In some embodiments, the standard of care comprises single antiplatelet therapy (SAPT).

In some embodiments, the standard of care comprises dual antiplatelet therapy (DAPT).

In some embodiments, the standard of care comprises dual antiplatelet therapy (DAPT) for 21 days, followed by single antiplatelet therapy (SAPT) thereafter.

In some aspects, the regimen used in the disclosed methods comprises milvexian and antiplatelet therapy.

In some embodiments, the antiplatelet therapy comprises single antiplatelet therapy.

In some embodiments, the single antiplatelet therapy is aspirin.

In some embodiments of the single antiplatelet therapy, aspirin is administered in an amount of 50-150 mg daily, such as, for example, one of: 50 mg, 55 mg, 60 mg, 65 mg, 70 mg, 75 mg, 80 mg, 81 mg, 85 mg, 90 mg, 95 mg, 100 mg, 105 mg, 110 mg, 115 mg, 120 mg, 125 mg, 130 mg, 135 mg, 140 mg, 145 mg, or 150 mg, daily.

In some embodiments of the single antiplatelet therapy, the aspirin is administered in an amount of 75-100 mg daily, such as, for example, one of: 75 mg, 80 mg, 81 mg, 85 mg, 90 mg, 95 mg, or 100 mg, daily.

In some embodiments of the single antiplatelet therapy, the aspirin is administered in an amount of 75 mg daily.

In some embodiments of the single antiplatelet therapy, the aspirin is administered in an amount of 81 mg daily.

In some embodiments of the single antiplatelet therapy, the aspirin is administered in an amount of 100 mg daily.

In other embodiments, the single antiplatelet therapy is a P2Y12 inhibitor.

In some embodiments of the single antiplatelet therapy, the P2Y12 inhibitor is clopidogrel, ticagrelor, ticlopidine, cangrelor, or prasugrel.

In some embodiments of the single antiplatelet therapy, the P2Y12 inhibitor is clopidogrel, ticagrelor, or prasugrel.

In some embodiments of the single antiplatelet therapy, the P2Y12 inhibitor is clopidogrel.

In some embodiments of the single antiplatelet therapy, the clopidogrel is administered in an amount of 75 mg to 600 mg daily, such as, for example, one of: 75 mg, 150 mg, 225 mg, 300 mg, 375 mg, 450 mg, 525 mg, or 600 mg daily.

In some embodiments of the single antiplatelet therapy, the P2Y12 inhibitor is ticagrelor.

In some embodiments of the single antiplatelet therapy, the P2Y12 inhibitor is ticlopidine.

In some embodiments of the single antiplatelet therapy, the P2Y12 inhibitor is cangrelor.

In some embodiments, the single antiplatelet therapy is ticlopidine.

In some embodiments, the ticlopidine is administered in amounts an amount of 250-500 mg daily, such as, for example, 150 mg, or 500 mg daily.

In other embodiments of the disclosed methods, the antiplatelet therapy comprises dual antiplatelet therapy.

In some embodiments, the dual antiplatelet therapy is aspirin and ticlopidine.

In some embodiments, the dual antiplatelet therapy is aspirin and a P2Y12 inhibitor.

In some embodiments of the dual antiplatelet therapy, the aspirin is administered in an amount of 50-150 mg daily, such as, for example, one of: 50 mg, 55 mg, 60 mg, 65 mg, 70 mg, 75 mg, 80 mg, 81 mg, 85 mg, 90 mg, 95 mg, 100 mg, 105 mg, 110 mg, 115 mg, 120 mg, 125 mg, 130 mg, 135 mg, 140 mg, 145 mg, or 150 mg, daily.

In some embodiments of the dual antiplatelet therapy, the aspirin is administered in an amount of 75-100 mg daily, such as, for example, one of 75 mg, 80 mg, 81 mg, 85 mg, 90 mg, 95 mg, or 100 mg, daily.

In some embodiments of the dual antiplatelet therapy, the aspirin is administered in an amount of 75 mg daily.

In some embodiments of the dual antiplatelet therapy, the aspirin is administered in an amount of 81 mg daily.

In some embodiments of the dual antiplatelet therapy, the aspirin is administered in an amount of 100 mg daily.

In some embodiments of the dual antiplatelet therapy, the P2Y12 inhibitor is clopidogrel, ticagrelor, ticlopidine, cangrelor, or prasugrel.

In some embodiments of the dual antiplatelet therapy, the P2Y12 inhibitor is clopidogrel, ticagrelor, or prasugrel.

In some embodiments of the dual antiplatelet therapy, the P2Y12 inhibitor is clopidogrel.

In some embodiments of the dual antiplatelet therapy, the clopidogrel is administered in an amount of 75 mg to 600 mg daily, such as, for example, one of: 75 mg, 150 mg, 225 mg, 300 mg, 375 mg, 450 mg, 525 mg, or 600 mg daily.

In some embodiments of the dual antiplatelet therapy, the clopidogrel is administered as a loading dose.

In some embodiments, the loading dose is between 300-600 mg daily.

In some embodiments of the dual antiplatelet therapy, the clopidogrel is administered in an amount of 75 mg daily.

In some embodiments of the dual antiplatelet therapy, the clopidogrel is administered in an amount of 150 mg daily.

In some embodiments of the dual antiplatelet therapy, the clopidogrel is administered in an amount of 225 mg daily.

In some embodiments of the dual antiplatelet therapy, the clopidogrel is administered in an amount of 300 mg daily.

In some embodiments of the dual antiplatelet therapy, the clopidogrel is administered in an amount of 375 mg daily.

In some embodiments of the dual antiplatelet therapy, the clopidogrel is administered in an amount of 450 mg daily.

In some embodiments of the dual antiplatelet therapy, the clopidogrel is administered in an amount of 525 mg daily.

In some embodiments of the dual antiplatelet therapy, the clopidogrel is administered in an amount of 600 mg daily.

In some embodiments, antiplatelet therapy comprises Aggrenox, i.e., a combination of aspirin and dipyridamole (persantine).

In some embodiments, the antiplatelet therapy comprises a combination of single antiplatelet therapy and dual antiplatelet therapy.

In some embodiments of the disclosed methods, the dual antiplatelet therapy is administered for up to 21 consecutive days, and single antiplatelet therapy is administered thereafter.

In some aspects, the regimen used in the methods of the disclosure are effective in preventing ischemic stroke in a human patient who has or has had an ischemic stroke or transient ischemic attack.

In some aspects, the regimen used in the methods of the disclosure are effective in reducing the risk of ischemic stroke in a human patient who has or has had an ischemic stroke or transient ischemic attack.

In some aspects, the regimen used in the methods of the disclosure are safe and effective in preventing ischemic stroke in a human patient who has or has had an ischemic stroke or transient ischemic attack.

In some aspects, the regimen used in the methods of the disclosure are safe and effective in reducing the risk of ischemic stroke in a human patient who has or has had an ischemic stroke or transient ischemic attack.

In some aspects, the regimen used in the methods of the disclosure are clinically proven safe and effective in preventing ischemic stroke in a human patient who has or has had an ischemic stroke or transient ischemic attack.

In some aspects, the regimen used in the methods of the disclosure are clinically proven safe and effective in reducing the risk of ischemic stroke in a human patient who has or has had an ischemic stroke or transient ischemic attack.

In some embodiments of the methods of the disclosure, the risk of ischemic stroke is reduced by the regimen as compared to administering placebo with the standard of care.

In some embodiments of the disclosed methods, the ischemic stroke relative risk by the regimen is about 0.9-0.6, such as, for example, about 0.9-0.85, about 0.85-0.8, about 0.85-0.75, about 0.85-0.7, about 0.85-0.65, about 0.8-0.75, about 0.8-0.7, about 0.8-0.65, about 0.8-0.6, about 0.75-0.7, about 0.75-0.65, about 0.75-0.6, about 0.7-0.65, about 0.7-0.6, about 0.65-0.6, or one of about 0.9, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.7, about 0.69, about 0.68, about 0.67, about 0.66, about 0.65, about 0.64, about 0.63, about 0.62, about 0.61, or about 0.6, compared to placebo with standard of care.

In some embodiments of the disclosed methods, the ischemic stroke relative risk reduction by the regimen is about 10-40%, such as, for example, about 10-15%, about 15-20%, about 15-25%, about 15-30%, about 15-35%, about 20-25%, about 20-30%, about 20-35%, about 20-40%, about 25-30%, about 25-35%, about 25-40%, about 30-35%, about 30-40%, about 35-40%, or one of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, compared to placebo with standard of care.

In some embodiments of the disclosed methods, the ischemic stroke absolute risk reduction by the regimen is about 1-10%, such as, for example, about 1-2%, about 1-3%, about 1-4%, about 1-5%, about 1-6%, about 1-7%, about 1-8%, about 1-9%, about 2-3%, about 2-4%, about 2-5%, about 2-6%, about 2-7%, about 2-8%, about 2-9%, about 2-10%, about 3-4%, about 3-5%, about 3-6%, about 3-7%, about 3-8%, about 3-9%, about 3-10%, about 4-5%, about 4-6%, about 4-7%, about 4-8%, about 4-9%, about 4-10%, about 5-6%, about 5-7%, about 5-8%, about 5-9%, about 5-10%, about 6-7%, about 6-8%, about 6-9%, about 6-10%, about 7-8%, about 7-9%, about 7-10%, about 8-9%, about 8-10%, or about 9-10%, or one of about 1%, about 2%, about 3%, about 4%, about 5%, about 6%, about 7%, about 8%, about 9%, or about 10%, compared to placebo with standard of care.

In some embodiments of the methods of the disclosure, the risk of ischemic stroke is reduced by the regimen compared to administering placebo with antiplatelet therapy.

In some embodiments of the disclosed methods, the ischemic stroke relative risk by the regimen is about 0.9-0.6, such as, for example, about 0.9-0.85, about 0.85-0.8, about 0.85-0.75, about 0.85-0.7, about 0.85-0.65, about 0.8-0.75, about 0.8-0.7, about 0.8-0.65, about 0.8-0.6, about 0.75-0.7, about 0.75-0.65, about 0.75-0.6, about 0.7-0.65, about 0.7-0.6, about 0.65-0.6, or one of about 0.9, about 0.89, about 0.88, about 0.87 about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.7, about 0.69, about 0.68, about 0.67, about 0.66, about 0.65, about 0.64, about 0.63, about 0.62, about 0.61, or about 0.6, compared to placebo with antiplatelet therapy.

In some embodiments of the disclosed methods, the ischemic stroke relative risk reduction by the regimen is about 10-40%, such as, for example, about 10-15%, about 15-20%, about 15-25%, about 15-30%, about 15-35%, about 20-25%, about 20-30%, about 20-35%, about 20-40%, about 25-30%, about 25-35%, about 25-40%, about 30-35%, about 30-40%, about 35-40%, or one of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, compared to placebo with antiplatelet therapy.

In some embodiments of the disclosed methods, the ischemic stroke absolute risk reduction by the regimen is about 1-10%, such as, for example, about 1-2%, about 1-3%, about 1-4%, about 1-5%, about 1-6%, about 1-7%, about 1-8%, about 1-9%, about 2-3%, about 2-4%, about 2-5%, about 2-6%, about 2-7%, about 2-8%, about 2-9%, about 2-10%, about 3-4%, about 3-5%, about 3-6%, about 3-7%, about 3-8%, about 3-9%, about 3-10%, about 4-5%, about 4-6%, about 4-7%, about 4-8%, about 4-9%, about 4-10%, about 5-6%, about 5-7%, about 5-8%, about 5-9%, about 5-10%, about 6-7%, about 6-8%, about 6-9%, about 6-10%, about 7-8%, about 7-9%, about 7-10%, about 8-9%, about 8-10%, or about 9-10%, or one of about 1%, about 2%, about 3%, about 4%, about 5%, about 6%, about 7%, about 8%, about 9%, or about 10%, compared to placebo with antiplatelet therapy.

In some embodiments of the disclosed methods, the risk of ischemic stroke decreases with the amount of milvexian administered in a dose-dependent manner.

In some aspects of the methods of the disclosure, the human patient to whom the regimen is administered does not suffer an ischemic stroke during the duration of the regimen.

In some embodiments, the duration of the regimen is from 4 weeks to 52 weeks, such as, for example, one of 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks.

In other embodiments, the duration of the regimen is one of at least 4 weeks, at least 6 weeks, at least 8 weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 34 weeks, at least 36 weeks, at least 38 weeks, at least 40 weeks, at least 42 weeks, at least 44 weeks, at least 46 weeks, at least 48 weeks, at least 50 weeks, or at least 52 weeks.

In some embodiments, the regimen is administered chronically, i.e., the duration of the regimen is the remainder of the patient's life.

In some embodiments, the duration of the regimen is one of: at least 0.5 year, at least 1 year, at least 2 years, at least 3 years, at least 4 years, at least 5 years, at least 6 years, at least 7 years, at least 8 years, at least 9 years, at least 10 years, at least 11 years, at least 12 years, at least 13 years, at least 14 years, at least 15 years, at least 16 years, at least 17 years, at least 18 years, at least 19 years, or at least 20 years.

In some aspects of the disclosed methods wherein the regimen is administered to a patient population, the percentage of patients in the population who experience an ischemic stroke within 90 days of beginning the regimen is about 10% or less, such as, for example, about 10% or less, about 9.9% or less, about 9.8% or less, about 9.7% or less, about 9.6% or less, about 9.5% or less, about 9.4% or less, about 9.3% or less, about 9.2% or less, about 9.1% or less, about 9% or less, about 8.9% or less, about 8.8% or less, about 8.7% or less, about 8.6% or less, about 8.5% or less, about 8.4% or less, about 8.3% or less, about 8.2% or less, about 8.1% or less, about 8% or less, about 7.9% or less, about 7.8% or less, about 7.7% or less, about 7.6% or less, about 7.5% or less, about 7.4% or less, about 7.3% or less, about 7.2% or less, about 7.1% or less, about 7% or less, about 6.9% or less, about 6.8% or less, about 6.7% or less, about 6.6% or less, about 6.5% or less, about 6.4% or less, about 6.3% or less, about 6.2% or less, about 6.1% or less, about 6% or less, about 5.9% or less, about 5.8% or less, about 5.7% or less, about 5.6% or less, about 5.5% or less, about 5.4% or less, about 5.3% or less, about 5.2% or less, about 5.1% or less, about 5% or less, about 4.9% or less, about 4.8% or less, about 4.7% or less, about 4.6% or less, about 4.5% or less, about 4.4% or less, about 4.3% or less, about 4.2% or less, about 4.1% or less, about 4.0% or less, about 3.9% or less, about 3.8% or less, about 3.7% or less, about 3.6% or less, about 3.5% or less, about 3.4% or less, about 3.3% or less, about 3.2% or less, about 3.1% or less, or about 3.0% or less.

In some embodiments of the methods of the disclosure, the Relative Risk of bleeding according to the Bleeding Academic Research Consortium (BARC) Type 3 or 5 criteria by the regimen is no greater than 3, such as, for example, no greater than one of: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with the standard of care.

In some embodiments of the methods of the disclosure, the Relative Risk of bleeding according to the Bleeding Academic Research Consortium (BARC) Type 3 or 5 criteria by the regimen is no greater than 3, such as, for example, no greater than one of: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the disclosed methods, the Relative Risk of serious bleeding according to the Bleeding Academic Research Consortium (BARC) Type 3 or 5 criteria is independent of the amount of milvexian administered.

In other embodiments of the disclosed methods, the human patient does not suffer serious bleeding according to the Bleeding Academic Research Consortium (BARC) Type 3 and 5 criteria.

In some embodiments of the methods of the disclosure, the Relative Risk of bleeding according to the Bleeding Academic Research Consortium (BARC) Type 2 criteria by the regimen is no greater than 2.6, such as, for example, no greater than one of: 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with standard of care.

In some embodiments of the methods of the disclosure, the Relative Risk of bleeding according to the Bleeding Academic Research Consortium (BARC) Type 2 criteria by the regimen is no greater than 2.6, such as, for example, no greater than one of: 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the disclosed methods, the Relative Risk of bleeding according to the Bleeding Academic Research Consortium (BARC) Type 2 criteria is independent of the amount of milvexian administered.

In other embodiments of the disclosed methods, the human patient does not suffer bleeding according to the Bleeding Academic Research Consortium (BARC) Type 2.

In some embodiments of the methods of the disclosure, the Relative Risk of bleeding according to the ISTH criteria (major bleeding or clinically-relevant non-major bleeding (CRNM)) by the regimen is no greater than 3, such as, for example, no greater than one of 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with standard of care.

In some embodiments of the methods of the disclosure, the Relative Risk of bleeding according to the ISTH criteria (major bleeding or clinically-relevant non-major bleeding (CRNM)) by the regimen is no greater than 3, such as, for example, no greater than one of 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some aspects of the disclosed methods wherein the regimen is administered to a patient population, the percentage of patients in the population who experience serious bleeding according to the Bleeding Academic Research Consortium (BARC) Type 3 or 5 criteria within 90 days is about 2% or less, such as, for example, one of about 2% or less, about 1.9% or less, about 1.8% or less, about 1.7% or less, about 1.6% or less, about 1.5% or less, about 1.4% or less, about 1.3% or less, about 1.2% or less, about 1.1% or less, about 1% or less, about 0.9% or less, about 0.8% or less, about 0.7% or less, about 0.6% or less, about 0.5% or less, about 0.4% or less, about 0.3% or less, about 0.2% or less, or about 0.1% or less.

In some aspects of the disclosed methods wherein the regimen is administered to a patient population, the percentage of patients in the population who experience bleeding according to the Bleeding Academic Research Consortium (BARC) Type 2 criteria within 90 days is about 3.5% or less, such as, for example, one of about 3.5% or less, about 3.4% or less, about 3.3% or less, about 3.2% or less, about 3.1% or less, about 3.0% or less, about 2.9% or less, about 2.8% or less, about 2.7% or less, about 2.6% or less, about 2.5% or less, about 2.4% or less, about 2.3% or less, about 2.2% or less, about 2.1% or less, about 2% or less, about 1.9% or less, about 1.8% or less, about 1.7% or less, about 1.6% or less, about 1.5% or less, about 1.4% or less, about 1.3% or less, about 1.2% or less, about 1.1% or less, about 1% or less, about 0.9% or less, about 0.8% or less, about 0.7% or less, about 0.6% or less, about 0.5% or less, about 0.4% or less, about 0.3% or less, about 0.2% or less, or about 0.1% or less.

In some aspects of the disclosed methods wherein the regimen is administered to a patient population, the percentage of patients in the population who experience bleeding according to the ISTH criteria (major bleeding or clinically-relevant non-major bleeding (CRNM)) criteria within 90 days is about 5% or less, such as, for example, one of about 5% or less, about 4.9% or less, about 4.8% or less, about 4.7% or less, about 4.6% or less, about 4.5% or less, about 4.4% or less, about 4.3% or less, about 4.2% or less, about 4.1% or less, about 4% or less, about 3.9% or less, about 3.8% or less, about 3.7% or less, about 3.6% or less, about 3.5% or less, about 3.4% or less, about 3.3% or less, about 3.2% or less, about 3.1% or less, about 3.0% or less, about 2.9% or less, about 2.8% or less, about 2.7% or less, about 2.6% or less, about 2.5% or less, about 2.4% or less, about 2.3% or less, about 2.2% or less, about 2.1% or less, about 2% or less, about 1.9% or less, about 1.8% or less, about 1.7% or less, about 1.6% or less, about 1.5% or less, about 1.4% or less, about 1.3% or less, about 1.2% or less, about 1.1% or less, about 1% or less, about 0.9% or less, about 0.8% or less, about 0.7% or less, about 0.6% or less, about 0.5% or less, about 0.4% or less, about 0.3% or less, about 0.2% or less, or about 0.1% or less.

In some aspects of the disclosed methods, administration of the regimen reduces the patient's FXI clotting activity.

In some embodiments, Factor XI (FXI) activity is measured using a modification of the activated partial thromboplastin time (aPTT) using Actin FS (Siemens Healthcare) on the Siemens BCS®XP analyzer. A 6-point calibration curve (˜5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS®XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FXI and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS®XP in saline.

In some embodiments of the methods of the disclosure, the administration of the regimen reduces the patient's FXI clotting activity, relative to baseline, in a dose-dependent manner.

In some embodiments of the methods of the disclosure, the administration of the regimen reduces the patient's FXI clotting activity by about 7% to about 70% relative to baseline.

In other aspects, in the methods of the disclosure, the administration of the regimen reduces the patient's FXI clotting activity by about 7% to about 43% relative to baseline.

In other aspects, in the methods of the disclosure, the administration of the regimen reduces the patient's FXI clotting activity by about 7% to about 20% relative to baseline.

In some embodiments, in the methods of the disclosure, the administration of the regimen reduces the patient's FXI clotting activity by about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, or about 68%, about 69%, or about 70%, relative to baseline.

In some embodiments, in the methods of the disclosure, the administration of the regimen reduces the patient's FXI clotting activity by about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, or about 43%, relative to baseline.

In other embodiments, in the methods of the disclosure, the administration of the regimen reduces the patient's FXI clotting activity by about 7%, about 8%, about 9%, about 10%, about 11%, about 12%, about 13%, about 14%, about 15%, about 16%, about 17%, about 18%, about 19%, or about 20%, relative to baseline.

In other embodiments, in the methods of the disclosure, the administration of the regimen reduces the patient's FXI clotting activity by an amount shown in Table 16 in Example 1.

In some aspects of the disclosed methods, administration of the regimen results in a dose-dependent prolongation of activated partial thromboplastin time (aPTT) relative to baseline.

In some aspects of the disclosed methods, administration of the regimen results in a prolongation of activated partial thromboplastin time (aPTT) ranging from about 27% to about 115% relative to baseline.

In some embodiments of the disclosed methods, the administration of the regimen results in a prolongation of activated partial thromboplastin time (aPTT) ranging from about 27% to about 115%, such as, for example, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87 about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, or about 115%, relative to baseline. As used here “baseline” refers to the patient's aPTT prior to any administration of the regimen.

In some aspects of the disclosed methods, administration of the regimen results in a prolongation of activated partial thromboplastin time (aPTT) ranging from about 27% to about 64% relative to baseline.

In some embodiments of the disclosed methods, the administration of the regimen results in a prolongation of activated partial thromboplastin time (aPTT) ranging from about 27% to about 64%, such as, for example, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, or about 64%, relative to baseline. As used here “baseline” refers to the patient's aPTT prior to any administration of the regimen.

In some embodiments of the disclosed methods, the administration of the regimen results in a prolongation of activated partial thromboplastin time (aPTT) in an amount shown in Table 15 in Example 1.

In some embodiments, aPTT is determined as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT).

In some aspects of the methods of the disclosure, the pharmaceutical composition comprising milvexian (or pharmaceutically acceptable salt or solvate thereof) is a solid oral pharmaceutical composition.

In some embodiments, the dose of milvexian, or pharmaceutically acceptable salt thereof, administered may be formulated as immediate release formulation.

In some embodiments, the dose of milvexian administered may be formulated as immediate release capsule formulation.

In some embodiments, the dose of milvexian administered may be formulated as an amorphous solid dispersion composition of milvexian in one or more polymers.

In some embodiments, the polymer in the amorphous solid dispersion is hypromellose acetate succinate (HPMCAS).

In some embodiments, the amorphous solid dispersion composition of milvexian in one or more polymers is prepared by spray drying.

In some embodiments, the amorphous solid dispersion composition of milvexian in one or more polymers comprises milvexian being molecularly dispersed in the one or more polymers.

In some embodiments of the methods of the disclosure, the solid oral pharmaceutical composition comprises a spray-dried amorphous solid dispersion (SDP) consisting essentially of milvexian free form and a pH-dependent enterosoluble polymer.

In some embodiments, the SDP is a spray-dried amorphous solid dispersion prepared according to the composition and method described in WO 2020210629. This SDP consists essentially of milvexian free form and hypromellose acetate succinate (HPMCAS-MG) in a weight ratio of 3:1 (milvexian: HPMCAS-MG).

In some embodiments of the methods of the disclosure, the SDP comprises the milvexian free form and the pH-dependent enterosoluble polymer in a weight ratio of 3:1 (milvexian:polymer).

In some embodiments of the methods of the disclosure, the pH-dependent enterosoluble polymer is cellulose acetate trimellitate (CAT), cellulose acetate phthalate (CAP), Hydroxypropyl methylcellulose phthalate (HPMCP), hydroxypropyl methylcellulose ES grade (HPMC ES), hydroxypropyl methyl cellulose acetate succinate (HPMC-AS) LF, LG, MF, MG or HF Grades such as Aqoat®, Polyvinyl acetate phthalate (PVAP) such as Sureteric® and Opadry® and Shellac resins such as SSB® Aquagold, or polyvinylpyrrolidone (PVP).

In some embodiments of the methods of the disclosure, the pH-dependent enterosoluble polymer is hydroxypropyl methyl cellulose-AS MG.

In some embodiments of the methods of the disclosure, the solid oral pharmaceutical composition further comprises a binder, such as, for example, microcrystalline cellulose (MCC), silicified microcrystalline cellulose (SMCC), or a combination thereof.

In some embodiments of the methods of the disclosure, the solid oral pharmaceutical composition further comprises a filler, such as, for example, lactose monohydrate.

In some embodiments of the methods of the disclosure, the solid oral pharmaceutical composition further comprises a disintegrant.

In some embodiments of the methods of the disclosure, the solid oral pharmaceutical composition further comprises a lubricant.

In some embodiments of the methods of the disclosure, the solid oral pharmaceutical composition is a tablet.

In some embodiments of the methods of the disclosure, the solid oral pharmaceutical composition is an immediate release tablet.

In some embodiments of the methods of the disclosure, the solid oral pharmaceutical composition is a direct compression tablet.

In some embodiments of the methods of the disclosure, the solid oral pharmaceutical composition is a roller compaction tablet.

In some embodiments of the methods of the disclosure, the solid oral pharmaceutical composition is a film-coated tablet.

In some embodiments of the methods of the disclosure, the film coating comprises polyvinyl alcohol, titanium dioxide, polyethylene glycol-polyvinyl alcohol graft copolymer, and talc.

In some embodiments of the methods of the disclosure, the film coating comprises polyethylene glycol-polyvinyl alcohol graft copolymer.

In some embodiments, the film coating comprises Opadry.

In some embodiments, the film coating comprises polyvinyl alcohol, iron oxide, macrogol (PEG) polyvinyl alcohol grafted copolymer, and talc.

In some embodiments, the film coating comprises OpadryQX 321A220063 Yellow, a film coating material that comprises polyvinyl alcohol, iron oxide, macrogol (PEG) polyvinyl alcohol grafted copolymer, and talc.

In some embodiments, the solid oral pharmaceutical composition is a tablet having the composition and/or properties shown in Table A:

TABLE A
Film-coated Tablet of Milvexian at 100 mg or 25 mg Strength

| | Amount | | |
|---|---|---|---|
| | | Amount | Amount |
| | | per dose | per dose |
| Component | wt. % | (25 mg) | (100 mg) |
| SDP^{b} | 22.22 | 33.33 | 133.3 |
| Silicified Microcrystalline cellulose | 43.07 | 64.6 | 258.4 |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 Yellow^{c} | 3.0 | 4.5 | 18.0 |
| | | 154.5 | 618 |
| Tablet hardness | | Average 60N | Average 140N |
| | | to 120N (Ph. | to 220N (Ph. |
| | | Eur. 2.9.8), | Eur. 2.9.8), |
| | | preferably 90N | preferably 180N |
| Tablet friability | | <0.5% | <0.5% |
| Disintegration time | | 13 | 18 |
| in water (seconds) | | | |

^{b}SDP: spray-dried amorphous solid dispersion prepared according to the composition and method described in WO 2020210629. SDP consists essentially of milvexian free form and hypromellose acetate succinate (HPMCAS-MG) in a weight ratio of 3:1 (milvexian:HPMCAS-MG).
^{c}OpadryQX 321A220063 Yellow: a film coating material comprises polyvinyl alcohol, iron oxide, macrogol (PEG) polyvinyl alcohol grafted copolymer, and talc.

In some embodiments in which the solid pharmaceutical composition is a tablet, the tablet has a disintegration time in water of less than 20 seconds.

In other embodiments in which the solid pharmaceutical composition is a tablet, the tablet has a disintegration time in water of less than 15 seconds.

In other embodiments in which the solid pharmaceutical composition is a tablet, the tablet has a disintegration time in water of less than 10 seconds.

In some embodiments of the methods of the disclosure, the solid pharmaceutical composition is a capsule.

In some aspects of the methods of the disclosure, the human patient to whom the pharmaceutical composition comprising milvexian (or a pharmaceutically acceptable salt or solvate thereof) is administered, is unable to swallow a tablet dosage form.

In some embodiments of the disclosed methods, the solid oral pharmaceutical composition is dispersed in an aqueous medium to form an aqueous dispersion for administration.

In some embodiments of the disclosed methods, the solid oral pharmaceutical composition is a tablet which is dispersed in an aqueous medium to form an aqueous dispersion for administration.

In some embodiments of the disclosed methods, the solid oral pharmaceutical composition is a tablet which is administered orally as an aqueous dispersion by dispersing the tablet in an aqueous medium in less than 1 minute.

In those embodiments in which the solid oral pharmaceutical composition is dispersed in an aqueous medium to form an aqueous dispersion, the aqueous medium is water, saline, phosphate buffer, vegetable juice, or a fruit juice, including, for example, apple sauce.

In those embodiments in which the solid oral pharmaceutical composition is a tablet which is dispersed in an aqueous medium to form an aqueous dispersion, the aqueous medium is water, saline, phosphate buffer, vegetable juice, or a fruit juice, including, for example, apple sauce.

In some embodiments of the disclosed methods in which the solid oral pharmaceutical composition is dispersed in an aqueous medium to form an aqueous dispersion for administration, the aqueous dispersion is administered to the human patient via a nasogastric tube or spoon.

In some embodiments of the disclosed methods in which the solid oral pharmaceutical composition is a tablet which is dispersed in an aqueous medium to form an aqueous dispersion for administration, the aqueous dispersion is administered to the human patient via a nasogastric tube or spoon.

In some embodiments of the disclosed methods, oral administration of the pharmaceutical composition comprising milvexian (or a pharmaceutically acceptable salt or solvate thereof) results in a milvexian plasma half-life ranging from about 13 hours to about 16 hours. In some embodiments of the disclosed methods, oral administration of the pharmaceutical composition comprising milvexian (or a pharmaceutically acceptable salt or solvate thereof) results in a milvexian plasma half-life ranging from about 13 hours to about 16 hours during repeat dosing. “Half-life (t_{1/2}),” as used herein, refers to apparent terminal elimination half-life having a mean value calculated using formula t_{1/2}=logₑ2/λz, where λz is the apparent terminal elimination rate-constant estimated by linear regression using the terminal log-linear phase of the logarithmic (log) transformed concentration vs time data. In some embodiments, half-life (t_{1/2}) has a mean value ranges from about 8 hours to about 16 hours. In some embodiments, half-life (t_{1/2}) has a mean value ranges from about 11 hours to about 16 hours. In a preferred embodiment, half-life (t_{1/2}) has a mean value ranges from about 13 hours to about 16 hours.

In some embodiments of the disclosed methods, administration of the pharmaceutical composition comprising milvexian (or a pharmaceutically acceptable salt or solvate thereof) to the human patient results in milvexian plasma concentrations reaching steady-state at about 3 days to 6 days. As used herein, the term “steady-state” refers to steady-state plasma concentrations as defined by regulatory agencies such as the U.S. Food & Drug Administration (FDA) and the European Medicines Agency (EMA).

In some embodiments of the disclosed methods, administration of the pharmaceutical composition comprising milvexian (or a pharmaceutically acceptable salt or solvate thereof) to the human patient results in milvexian plasma concentrations reaching steady-state in about 3 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 4 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 5 days.

In some embodiments of the disclosed methods, administration of the pharmaceutical composition comprising milvexian (or a pharmaceutically acceptable salt or solvate thereof) to the human patient results in milvexian plasma concentrations reaching steady-state in about 6 days.

In some embodiments of the disclosed methods, the pharmaceutical composition comprising milvexian (or a pharmaceutically acceptable salt or solvate thereof) is administered without regard to the timing of food intake.

In some aspects, the regimen used in the methods of the disclosure may be administered using separate dosage forms for milvexian and the components of the antiplatelet therapy, or using a combination dosage form containing both milvexian and the components of the antiplatelet therapy. These dosage forms further comprises a pharmaceutically acceptable excipient. If a combination dosage form is used in a method in which milvexian is administered twice per day, the invention includes using a combination dosage form for one of the daily administrations and using a dosage form containing milvexian without antiplatelet therapy as the second dosage form in the daily regimen. The invention also includes using a combination dosage form containing antiplatelet therapy and milvexian for both of the twice daily administrations.

“Combinations” mean for the purposes of the invention not only dosage forms which comprise all the components (so-called fixed dose combinations), and combination packs or kits which comprise the components separate from one another in a package together, optionally with instructions for use according to one of the methods of treatment or methods of reducing the risk of a disorder or injury disclosed herein, but also components administered simultaneously or sequentially as long as they are employed for the prophylaxis and/or treatment of the same disease. The combinations of the invention may also be used in the manufacture of a medicament.

The individual active ingredients of the combinations are known in the art. They can be employed in doses that, if used alone without the other elements of the claimed combination, are subtherapeutically effective doses.

Formulations of milvexian are described in U.S. Pat. No. 9,453,018, WO2021207659, WO2022081473, WO2020210629, which are hereby incorporated by reference in their entireties. Combination dosage forms comprising both milvexian and antiplatelet therapy can be made by following these examples for formulations of milvexian, well-known formulations of antiplatelet therapy, and the understanding of the person of ordinary skill in pharmaceutical formulation.

The methods disclosed herein may be used in combination with a further cardiovascular medication or medications. For example, the methods and combinations involving milvexian and antiplatelet therapy disclosed herein may be used with one or more of the following further cardiovascular medications: (1) angiotensin-converting enzyme (ACE) inhibitors, such as benazepril (Lotensin®), captopril (Capote®), enalapril (Vasotec®), fosinopril (Monopril®), lisinopril (Prinivil®, Zestril®), moexipril (Univasc®), perindopril (Aceon®), quinapril (Accupril®), ramipril (Altace®), and trandolapril (Mavik®); (2) angiotensin II (All) receptor antagonists, such as embusartan, losartan (Cozaar®), valsartan (Diovan®), irbesartan (Avapro®), candesartan (Atacand®), eprosartan (Teveten®) and telmisartan (Micardis®); (3) statins, such as atorvastatin (Lipitor®), rosuvastatin (Crestor®), and simvastatin (Zocor® and FloLipid®); (4) nicotinic acids, such as lovastatin (Advicor®), (5) cholesterol absorption inhibitors such as ezetimibe/simvastatin (Vytorin®); and (6) beta-adrenergic blocking agents such as acebutolol (Sectral®), atenolol (Tenormin®), betaxolol (Kerlone®), bisoprolol/hydrochlorothiazide (Ziac®), bisoprolol (Zebeta®), metoprolol (Lopressor®, Toprol® XL), nadolol (Corgard®), propranolol (Inderal®), and sotalol (Betapace®).

All usual administration forms are suitable for administering the regimen of the disclosure. Administration preferably takes place orally, lingually, sublingually, buccally, rectally, topically or parenterally (i.e. avoiding the intestinal tract, i.e. intravenous, intracardiac, intracutaneous, subcutaneous, transdermal, intraperitoneal or intramuscular), preferably orally for both milvexian and antiplatelet therapy.

In some aspects, the disclosure provides milvexian (or pharmaceutically acceptable salt or solvate thereof) for use in a methods disclosed herein.

In other aspects, the disclosure provides compositions comprising milvexian (or pharmaceutically acceptable salt or solvate thereof) for use in the methods disclosed herein.

The present invention includes pharmaceutical products, or dosage forms, which comprise, in addition to non-toxic, inert pharmaceutically suitable excipients and/or carriers, milvexian, antiplatelet therapy, or both milvexian and antiplatelet therapy. Processes for producing these pharmaceutical products are also encompassed. These pharmaceutical products may comprise, in addition to milvexian, antiplatelet therapy, or both milvexian and antiplatelet therapy, further active pharmaceutical ingredients such as the cardiovascular medications discussed herein for use with the combinations and methods of use of the present invention.

As used herein, the term “dosage form” refers to a physical manifestation containing milvexian and one or more pharmaceutically acceptable excipients that deliver a dose of milvexian to a patient. In some embodiments, the amount of milvexian that is in the dosage form is preferably from about 25 mg to about 100 mg. More preferably such dosage form contains about 25 mg, about 50 mg, about 75 mg or about 100 mg milvexian. Most preferably such dosage form contains about 25 mg milvexian. In some embodiments, the dosage form is preferably in unit dosage forms such as tablets, pills, capsules, powders, granules, sterile parenteral solutions or suspension; for oral parenteral, intranasal, sublingual administration. In some embodiments, the pharmaceutical product is prepared according to the compositions and methods described in WO2020210629.

As used herein, the term “pharmaceutical product” refers to a dosage form containing milvexian that is administered multiple times per day to a patient in an amount that provides for a decrease of about 27% to about 36% in the incidence rate of clinical ischemic stroke events for 90 days in the patient compared to the placebo group.

In some embodiments, the pharmaceutical product is a dosage form containing milvexian that is administered multiple times per day to a patient in an amount that provides a Relative Risk Reduction of about 27% to about 36% in the incidence of clinical ischemic stroke for 90 days, relative to placebo with the standard of care.

In some embodiments, the pharmaceutical product is a dosage form containing milvexian that is administered multiple times per day to a patient in an amount that provides a Relative Risk Reduction of about 27% to about 36% in the incidence of clinical ischemic stroke for 90 days compared to placebo with antiplatelet therapy.

In some embodiments, the amount of milvexian in the dosage form is from about 20 mg to less than 200 mg.

In some embodiments, the amount of milvexian in the dosage form is from about 25 mg to less than 200 mg.

In some embodiments, the amount of milvexian in the dosage form is from about 25 mg to about 100 mg.

In some embodiments, the amount of milvexian in the dosage form is from about 25 mg to about 50 mg.

In some embodiments, the amount of milvexian in the dosage form is from about 75 mg to about 100 mg.

In some embodiments, the amount of milvexian in the dosage form is from about 20 mg.

In some embodiments, the amount of milvexian in the dosage form is from about 25 mg.

In some embodiments, the amount of milvexian in the dosage form is from about 50 mg.

In some embodiments, the amount of milvexian in the dosage form is from about 75 mg.

In some embodiments, the amount of milvexian in the dosage form is from about 100 mg.

In some embodiments, the amount of milvexian in the dosage form is from about 125 mg.

In some embodiments, the amount of milvexian in the dosage form is from about 150 mg.

In some embodiments, the amount of milvexian in the dosage form is from about 175 mg.

In some embodiments, the dosage form is administered two times per day to a patient at risk for clinical ischemic stroke.

In an embodiment, the present disclosure provides a pharmaceutical product that is administered twice daily to a patient who has or has had an ischemic stroke or a transient ischemic attack.

In some embodiments, for any of the pharmaceutical products described herein, the amount of milvexian in the pharmaceutical product in each administration is in a range from about 25 mg to about 100 mg.

In some embodiments, for any of the pharmaceutical products described herein, the amount of milvexian in the pharmaceutical product in each administration is about 25 mg.

In some embodiments, for any of the pharmaceutical products described herein, wherein the pharmaceutical product causes an about 30% relative risk reduction (RRR) in the occurrence of clinical ischemic stroke events in the patient without increased bleeding versus the placebo group.

In an embodiment, this disclosure provides a method of preventing adverse cerebrovascular events or adverse cardiovascular events in a human patient with atherosclerosis, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof; wherein the pharmaceutical composition is administered twice daily.

In some embodiments, the antiplatelet therapy is a P2Y12 inhibitor.

In some embodiments, the P2Y12 inhibitor is clopidogrel, ticagrelor, ticlopidine, cangrelor, or prasugrel.

In some embodiments, the P2Y12 inhibitor is clopidogrel, ticagrelor, or prasugrel.

In some embodiments, the antiplatelet therapy is aspirin.

In some embodiments, the human patient is treated with aspirin and clopidogrel combination therapy from day 1 to day 21, followed by aspirin monotherapy for at least 90 days.

In some embodiments, the patient is treated with aspirin and/or clopidogrel without milvexian dose adjustment.

In some embodiments, the adverse cerebrovascular events or adverse cardiovascular events comprises one or more of stroke, heart attack, or death.

In some embodiments, the adverse cerebrovascular events or adverse cardiovascular events occur in an organ selected from heart, brain, limb, blood supply system, or vessel.

In some embodiments, the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.

In some embodiments, the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.

In some embodiments, the adverse cerebrovascular events or adverse cardiovascular events are selected from the group consisting of arrhythmogenic cardiomyopathy (ACM), nonfatal myocardial infarction, ischemic stroke, and combinations thereof.

In some embodiments, the adverse cerebrovascular events or adverse cardiovascular events comprises cardiovascular death.

In some embodiments, the adverse cerebrovascular events or adverse cardiovascular events comprises arrhythmogenic cardiomyopathy (ACM).

In an embodiment, this disclosure provides a method for preventing adverse cardiovascular events in a human patient diagnosed with atherosclerosis, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof,

In some embodiments, the pharmaceutical composition is administered twice daily; and

In some embodiments, the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

In some embodiments, the administration of the regimen results in a relative risk reduction (RRR) in the occurrence of symptomatic venous thromboembolism (VTE: (pulmonary embolism (PE), deep vein thrombosis (DVT) in the patient of at least 25%, without increased bleeding, versus a placebo group.

In some embodiments, the adverse cardiovascular event is one or more of CV death, MI, or ischemic stroke.

A method for reducing incidence rate of the one or more of adverse thrombotic event selected from new ischemic stroke, MI, or all-cause death in a human patient diagnosed with atherosclerosis, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

In some embodiments, the administration of the regimen results in a relative risk is 0.85 or less relative to the placebo.

A method for preventing ischemic stroke in a human patient diagnosed with atherosclerosis, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

In some embodiments, the administration of the regimen results in a relative risk reduction (RRR) in the occurrence of clinical ischemic stroke events in the patient is at least 25%, without increased bleeding, versus a placebo group.

In some embodiments, the clinical benefit of reducing the incidence rates of the clinical ischemic stroke by the regimen is maintained throughout a treatment period of 90 days.

In some embodiments, the administration of the regimen reduces the patient's FM clotting activity by about 7% to about 20% relative to baseline.

In some embodiments, the administration of the regimen results is a prolongation of activated partial thromboplastin time (aPTT) ranging from about 27% to about 64% relative to baseline.

In some embodiments, the administration does not result in a statistically significant increase in major bleeding complications.

In some embodiments, the pharmaceutical composition comprising milvexian (or pharmaceutically acceptable salt or solvate thereof) is a solid oral pharmaceutical composition. In some embodiments, the solid oral pharmaceutical composition is a tablet. In some embodiments, the tablet is an immediate release tablet. In some embodiments, the tablet has a disintegration time in water of less than 20 seconds.

In some embodiments, the administration results in a milvexian plasma half-life ranging from about 13 hours to about 16 hours. In some embodiments, the administration results in a milvexian plasma half-life ranging from about 13 hours to about 16 hours during repeat dosing.

In some embodiments, the administration results in milvexian plasma concentration reaching steady-state at about 3 days to 6 days.

In some embodiments, the pharmaceutical composition comprising milvexian (or pharmaceutically acceptable salt or solvate thereof) is administered without regard to the timing of food intake.

In some embodiments, the human patient is unable to swallow a tablet dosage form.

In some embodiments, the tablet is dispersed in an aqueous medium to form an aqueous dispersion. In some embodiments, the aqueous medium is water, saline, phosphate buffer, vegetable juice, or fruit juice, including, for example, apple sauce. In some embodiments, the aqueous dispersion is administered to the human patient via a nasogastric tube or spoon. In some embodiments, the pharmaceutical composition is administered orally as an aqueous dispersion by dispersing the oral tablet in an aqueous medium in less than 1 minute.

In some embodiments, for any of the pharmaceutical products described herein, wherein the clinical benefit of reducing the incidence rates of the clinical ischemic stroke by the pharmaceutical product is maintained throughout the treatment period of 90 days.

The disclosure is also directed to the following aspects:

Aspect 1. A method of preventing ischemic stroke in a human patient who has or has had an ischemic stroke or transient ischemic attack, the method comprising administering to the human patient a regimen comprising milvexian (or pharmaceutically acceptable salt or solvate thereof) in an amount of 25 mg once daily or in an amount of 12.5 mg to 200 mg twice daily; and the standard of care; wherein the regimen is effective in preventing an ischemic stroke in the human patient.

Aspect 2. A method of reducing the risk of ischemic stroke in a human patient who has or has had an ischemic stroke or transient ischemic attack, the method comprising administering to the human patient a regimen comprising milvexian (or pharmaceutically acceptable salt or solvate thereof) in an amount of 25 mg once daily or in an amount of 12.5 mg to 200 mg twice daily; and the standard of care; wherein the regimen is effective in reducing the risk of ischemic stroke in the human patient, relative to placebo with the standard of care.

Aspect 3. The method of aspect 1 or aspect 2, wherein the standard of care comprises antiplatelet therapy.

Aspect 4. The method of aspect 3, wherein the antiplatelet therapy comprises single antiplatelet therapy.

Aspect 5. The method of aspect 4, wherein the single antiplatelet therapy is aspirin.

Aspect 6. The method of aspect 5, wherein the aspirin is administered in an amount of 75-100 mg daily.

Aspect 7. The method of aspect 6, wherein the aspirin is administered in an amount of 75 mg daily.

Aspect 8. The method of aspect 6, wherein aspirin is administered in an amount of 81 mg daily.

Aspect 9. The method of aspect 6, wherein aspirin is administered in an amount of 100 mg daily.

Aspect 10. The method of aspect 4, wherein the single antiplatelet therapy is a P2Y12 inhibitor.

Aspect 11. The method of aspect 10, wherein the P2Y12 inhibitor is clopidogrel, ticagrelor, or prasugrel.

Aspect 12. The method of aspect 11, wherein the P2Y12 inhibitor is clopidogrel.

Aspect 13. The method of aspect 12, wherein the clopidogrel is administered in an amount of 75 mg daily.

Aspect 14. The method of aspect 4, wherein the single antiplatelet therapy is ticlopidine.

Aspect 15. The method of aspect 14, wherein the ticlopidine is administered in an amount of 250-500 mg daily.

Aspect 16. The method of aspect 3, wherein the antiplatelet therapy comprises dual antiplatelet therapy.

Aspect 17. The method of aspect 16, wherein the dual antiplatelet therapy is aspirin and a P2Y12 inhibitor.

Aspect 18. The method of aspect 17, wherein the aspirin is administered in an amount of 75-100 mg daily.

Aspect 19. The method of aspect 18, wherein the aspirin is administered in an amount of 75 mg daily.

Aspect 20. The method of aspect 18, wherein aspirin is administered in an amount of 81 mg daily.

Aspect 21. The method of aspect 18, wherein aspirin is administered in an amount of 100 mg daily.

Aspect 22. The method of any one of aspects 17 to 21, wherein the P2Y12 inhibitor is clopidogrel, ticagrelor, or prasugrel.

Aspect 23. The method of aspect 22, wherein the P2Y12 inhibitor is clopidogrel.

Aspect 24. The method of aspect 23, wherein the clopidogrel is administered in an amount of 75 mg daily.

Aspect 25. The method of aspect 3, wherein the antiplatelet therapy comprises Aggrenox, i.e., a combination of aspirin and dipyridamole (persantine).

Aspect 26. The method of any one of aspects 1-25, wherein the milvexian is administered in an amount of 25 mg once daily.

Aspect 27. The method of any one of aspects 1-25, wherein the milvexian is administered in an amount of 25 mg twice daily.

Aspect 28. The method of any one of aspects 1-25, wherein the milvexian is administered in an amount of 50 mg twice daily.

Aspect 29. The method of any one of aspects 1-25, wherein the milvexian is administered in an amount of 100 mg twice daily.

Aspect 30. The method of any one of aspects 1-25, wherein the milvexian is administered in an amount of 200 mg twice daily.

Aspect 31. The method of any one of aspects 1-25, wherein the milvexian is administered in an amount of less than 200 mg twice daily.

Aspect 32. The method of any one of aspects 1-31, wherein the risk of ischemic stroke is reduced compared to administering the placebo with standard of care.

Aspect 33. The method of any one of aspects 1-32, wherein the risk of ischemic stroke is reduced compared to administering placebo with antiplatelet therapy.

Aspect 34. The method of aspect 32 or aspect 33, wherein the relative risk is about 0.9-0.6.

Aspect 35. The method of aspect 32 or aspect 33, wherein the relative risk reduction is about 10-40%.

Aspect 36. The method of any one of the preceding aspects, wherein the risk of ischemic stroke decreases with the amount of milvexian administered in a dose-dependent manner.

Aspect 37. A method of preventing ischemic stroke in a human patient who has or has had an ischemic stroke or transient ischemic attack, the method comprising administering to the human patient a regimen comprising milvexian (or pharmaceutically acceptable salt or solvate thereof) in an amount 25 mg twice daily; and the standard of care; wherein the regimen is effective in preventing an ischemic stroke in the human patient.

Aspect 38. A method of reducing the risk of ischemic stroke in a human patient who has or has had an ischemic stroke or transient ischemic attack, the method comprising administering to the human patient a regimen comprising milvexian (or pharmaceutically acceptable salt or solvate thereof) in an amount of 25 mg twice daily; and the standard of care; wherein the regimen is effective in reducing the risk of ischemic stroke in the human patient, relative to placebo with the standard of care.

Aspect 39. The method of any one of aspects 1-38, wherein the relative risk of bleeding according to the Bleeding Academic Research Consortium (BARC) Type 3 and 5 criteria is no greater than 3, compared to placebo with the standard of care.

Aspect 40. The method of any one of aspects 1-39, wherein the relative risk of bleeding according to the Bleeding Academic Research Consortium (BARC) Type 3 and 5 criteria is no greater than 3, compared to placebo with antiplatelet therapy.

Aspect 41. The method of any one of aspects 1-40, wherein the risk of serious bleeding according to the Bleeding Academic Research Consortium (BARC) Type 3 and 5 criteria is independent of the amount of milvexian administered.

Aspect 42. The method of any one of aspects 1-41, wherein the relative risk of bleeding according to the Bleeding Academic Research Consortium (BARC) Type 2 criteria is no greater than 2.6, compared to placebo with the standard of care.

Aspect 43. The method of any one of aspects 1-42, wherein the relative risk of bleeding according to the Bleeding Academic Research Consortium (BARC) Type 2 criteria is no greater than 2.6, compared to placebo with antiplatelet therapy.

Aspect 44. The method of any one of aspects 1-43, wherein the risk of bleeding according to the Bleeding Academic Research Consortium (BARC) Type 2 criteria is independent of the amount of milvexian administered.

Aspect 45. The method of any one of aspects 1-44, wherein the relative risk of bleeding according to the ISTH criteria (major bleeding or clinically-relevant non-major bleeding (CRNM)) is no greater than 3, compared to placebo with the standard of care.

Aspect 46. The method of any one of aspects 1-45, wherein the relative risk of bleeding according to the ISTH criteria (major bleeding or clinically-relevant non-major bleeding (CRNM)) is no greater than 3, compared to placebo with antiplatelet therapy.

Aspect 47. A method of treating a human patient who has or has had an ischemic stroke or transient ischemic attack, comprising administering to the human patient a regimen comprising milvexian (or pharmaceutically acceptable salt or solvate thereof) in an amount of 25 mg once daily, or 12.5 mg to 200 mg twice daily and the standard of care; wherein the regimen is effective in reducing the risk of ischemic stroke in the human patient and wherein the human patient does not suffer an ischemic stroke during the duration of the regimen.

Aspect 48. A method of treating a human patient who has or has had an ischemic stroke or transient ischemic attack, comprising administering to the human patient a regimen comprising milvexian (or pharmaceutically acceptable salt or solvate thereof) in an amount 25 mg twice daily and the standard of care; wherein the regimen is effective in reducing the risk of ischemic stroke in the human patient and wherein the human patient does not suffer an ischemic stroke during the duration of the regimen.

Aspect 49. The method of aspect 47 or aspect 48, wherein the standard of care comprises antiplatelet therapy.

Aspect 50. The method of aspect 49, wherein the antiplatelet therapy comprises single antiplatelet therapy.

Aspect 51. The method of aspect 50, wherein the single antiplatelet therapy is aspirin.

Aspect 52. The method of aspect 51, wherein the aspirin is administered in an amount of 75-100 mg daily.

Aspect 53. The method of aspect 51, wherein the aspirin is administered in an amount of 75 mg daily.

Aspect 54. The method of aspect 51, wherein aspirin is administered in an amount of 81 mg daily.

Aspect 55. The method of aspect 51, wherein aspirin is administered in an amount of 100 mg daily.

Aspect 56. The method of aspect 50, wherein the single antiplatelet therapy is a P2Y12 inhibitor.

Aspect 57. The method of aspect 56, wherein the P2Y12 inhibitor is clopidogrel, ticagrelor, or prasugrel.

Aspect 58. The method of aspect 57, wherein the P2Y12 inhibitor is clopidogrel.

Aspect 59. The method of aspect 58, wherein the clopidogrel is administered in an amount of 75 mg daily.

Aspect 60. The method of aspect 50, wherein the single antiplatelet therapy is ticlopidine.

Aspect 61. The method of aspect 60, wherein the ticlopidine is administered in an amount of 250-500 mg daily.

Aspect 62. The method of aspect 49, wherein the antiplatelet therapy comprises dual antiplatelet therapy.

Aspect 63. The method of aspect 62, wherein the dual antiplatelet therapy is aspirin and a P2Y12 inhibitor.

Aspect 64. The method of aspect 63, wherein the aspirin is administered in an amount of 75-100 mg daily.

Aspect 65. The method of aspect 64, wherein the aspirin is administered in an amount of 75 mg daily.

Aspect 66. The method of aspect 64, wherein aspirin is administered in an amount of 81 mg daily.

Aspect 67. The method of aspect 64, wherein aspirin is administered in an amount of 100 mg daily.

Aspect 68. The method of any one of aspects 63 to 67, wherein the P2Y12 inhibitor is clopidogrel, ticagrelor, or prasugrel.

Aspect 69. The method of aspect 68, wherein the P2Y12 inhibitor is clopidogrel.

Aspect 70. The method of aspect 69, wherein the clopidogrel is administered in an amount of 75 mg daily.

Aspect 71. The method of aspect 49, wherein the antiplatelet therapy comprises Aggrenox, i.e., a combination of aspirin and dipyridamole (persantine).

Aspect 72. The method of any one of aspects 47, or 49-71, wherein the milvexian is administered in an amount of 25 mg once daily.

Aspect 73. The method of any one of aspects 47-71, wherein the milvexian is administered in an amount of 25 mg twice daily.

Aspect 74. The method of any one of aspects 47, or 49-71, wherein the milvexian is administered in an amount of 50 mg twice daily.

Aspect 75. The method of any one of aspects 47, or 49-71, wherein the milvexian is administered in an amount of 100 mg twice daily.

Aspect 76. The method of any one of aspects 47, or 49-71, wherein the milvexian is administered in an amount of less than 200 mg twice daily.

Aspect 77. The method of any one of aspects 47, or 49-71, wherein the milvexian is administered in an amount of 200 mg twice daily.

Aspect 78. The method of anyone of aspects 47-77, wherein the regimen is administered for at least 12 weeks.

Aspect 79. The method of anyone of aspects 47-77, wherein the regimen is administered for at least 24 weeks.

Aspect 80. The method of anyone of aspects 47-77, wherein the regimen is administered for at least 36 weeks.

Aspect 81. The method of anyone of aspects 47-77, wherein the regimen is administered for at least 48 weeks.

Aspect 82. The method of any one of aspects 47-81, wherein the relative risk of bleeding according to the Bleeding Academic Research Consortium (BARC) Type 3 and 5 criteria is no greater than 3, compared to placebo with the standard of care.

Aspect 83. The method of any one of aspects 47-81, wherein the relative risk of bleeding according to the Bleeding Academic Research Consortium (BARC) Type 3 and 5 criteria is no greater than 3, compared to placebo with antiplatelet therapy.

Aspect 84. The method of any one of aspects 47-81, wherein the risk of serious bleeding according to the Bleeding Academic Research Consortium (BARC) Type 3 and 5 criteria is independent of the amount of milvexian administered.

Aspect 85. The method of any one of aspects 47-84, wherein the relative risk of bleeding according to the Bleeding Academic Research Consortium (BARC) Type 2 criteria is no greater than 2.6, compared to placebo with the standard of care.

Aspect 86. The method of any one of aspects 47-85, wherein the relative risk of bleeding according to the Bleeding Academic Research Consortium (BARC) Type 2 criteria is no greater than 2.6, compared to placebo with antiplatelet therapy.

Aspect 87. The method of any one of aspects 47-86, wherein the risk of bleeding according to the Bleeding Academic Research Consortium (BARC) Type 2 criteria is independent of the amount of milvexian administered.

Aspect 88. The method of any one of aspects 47-87, wherein the relative risk of bleeding according to the ISTH criteria (major bleeding or clinically-relevant non-major bleeding (CRNM)) is no greater than 3, compared to placebo with the standard of care.

Aspect 89. The method of any one of aspects 47-88, wherein the relative risk of bleeding according to the ISTH criteria (major bleeding or clinically-relevant non-major bleeding (CRNM)) is no greater than 3, compared to placebo with antiplatelet therapy.

Aspect 90. The method of any one of aspects 47-88, wherein the human patient does not suffer bleeding according to the Bleeding Academic Research Consortium (BARC) Type 3 and 5 criteria.

Aspect 91. A method of treating a patient population wherein the patients have or have had an ischemic stroke or a transient ischemic attack, comprising administering to the patients in the population for a regimen comprising milvexian (or pharmaceutically acceptable salt or solvate thereof) in an amount of 25 mg once daily or in an amount of 12.5 mg to 200 mg twice daily; and the standard of care; wherein the percentage of patients in the population who experience an ischemic stroke within 90 days of beginning the regimen is less than 10%.

Aspect 92. A method of treating a patient population wherein the patients have or have had an ischemic stroke or a transient ischemic attack, comprising administering to the patients in the population for a regimen comprising milvexian (or pharmaceutically acceptable salt or solvate thereof) in an amount of 25 mg twice daily; and the standard of care; wherein the percentage of patients in the population who experience an ischemic stroke within 90 days of beginning the regimen is less than 10%.

Aspect 93. The method of aspect 91 or aspect 92, wherein the percentage of patients in the population who experience an ischemic stroke within 90 days of beginning the regimen is less than 4%.

Aspect 94. The method of any one of aspects 91-93, wherein the standard of care comprises antiplatelet therapy.

Aspect 95. The method of aspect 94, wherein the antiplatelet therapy is single antiplatelet therapy.

Aspect 96. The method of aspect 95, wherein the single antiplatelet therapy is aspirin.

Aspect 97. The method of aspect 96, wherein the aspirin is administered in an amount of 75-100 mg daily.

Aspect 98. The method of aspect 97, wherein the aspirin is administered in an amount of 75 mg daily.

Aspect 99. The method of aspect 97, wherein aspirin is administered in an amount of 81 mg daily.

Aspect 100. The method of aspect 97, wherein aspirin is administered in an amount of 100 mg daily.

Aspect 101. The method of aspect 95, wherein the single antiplatelet therapy is a P2Y12 inhibitor.

Aspect 102. The method of aspect 101, wherein the P2Y12 inhibitor is clopidogrel, ticagrelor, or prasugrel.

Aspect 103. The method of aspect 102, wherein the P2Y12 inhibitor is clopidogrel.

Aspect 104. The method of aspect 103, wherein the clopidogrel is administered in an amount of 75 mg daily.

Aspect 105. The method of aspect 95, wherein the single antiplatelet therapy is ticlopidine.

Aspect 106. The method of aspect 105, wherein the ticlopidine is administered in an amount of 250-500 mg daily.

Aspect 107. The method of aspect 94, wherein the antiplatelet therapy is dual antiplatelet therapy.

Aspect 108. The method of aspect 107, wherein the dual antiplatelet therapy is aspirin and a P2Y12 inhibitor.

Aspect 109. The method of aspect 108, wherein the aspirin is administered in an amount of 75-100 mg daily.

Aspect 110. The method of aspect 109, wherein the aspirin is administered in an amount of 75 mg daily.

Aspect 111. The method of aspect 109, wherein aspirin is administered in an amount of 81 mg daily.

Aspect 112. The method of aspect 109, wherein aspirin is administered in an amount of 100 mg daily.

Aspect 113. The method of any one of aspects 108 to 112, wherein the P2Y12 inhibitor is clopidogrel, ticagrelor, or prasugrel.

Aspect 114. The method of aspect 113, wherein the P2Y12 inhibitor is clopidogrel.

Aspect 115. The method of aspect 114, wherein the clopidogrel is administered in an amount of 75 mg daily.

Aspect 116. The method of aspect 94, wherein the antiplatelet therapy comprises Aggrenox, i.e., a combination of aspirin and dipyridamole (persantine).

Aspect 117. The method of any one of aspects 91, or 93-116, wherein the milvexian is administered in an amount of 25 mg once daily.

Aspect 118. The method of any one of aspects 91-116, wherein the milvexian is administered in an amount of 25 mg twice daily.

Aspect 119. The method of any one of aspects 91, or 93-116, wherein the milvexian is administered in an amount of 50 mg twice daily.

Aspect 120. The method of any one of aspects 91, or 93-116, wherein the milvexian is administered in an amount of 100 mg twice daily.

Aspect 121. The method of any one of aspects 91, or 93-116, wherein the milvexian is administered in an amount of less than 200 mg twice daily.

Aspect 122. The method of any one of aspects 91, or 93-116, wherein the milvexian is administered in an amount of 200 mg twice daily.

Aspect 123. The method of any one of aspects 91-122, wherein the percentage of patients in the population who experience serious bleeding according to the Bleeding Academic Research Consortium (BARC) Type 3 or 5 criteria within 90 days is about 2% or less.

Aspect 124. The method of any one of aspects 91-122, wherein the percentage of patients in the population who experience bleeding according to the Bleeding Academic Research Consortium (BARC) Type 2 criteria within 90 days is about 3.5% or less.

Aspect 125. The method of any one of aspects 91-122, wherein the percentage of patients in the population who experience bleeding according to the ISTH criteria (major bleeding or clinically-relevant non-major bleeding (CRNM)) criteria within 90 days is about 5% or less.

Aspect 126. A method of preventing ischemic stroke in an adult patient after an acute ischemic stroke or high risk transient ischemic attack, wherein the method comprises administering to the adult patient a regimen comprising milvexian (or pharmaceutically acceptable salt or solvate thereof) in an amount of 25 mg once daily or in an amount of 12.5 mg to 200 mg twice daily.

Aspect 127. A method of reducing the risk of ischemic stroke in an adult patient after an acute ischemic stroke or high risk transient ischemic attack, wherein the method comprises administering to the adult patient a regimen comprising milvexian (or pharmaceutically acceptable salt or solvate thereof) in an amount of 25 mg once daily or in an amount of 12.5 mg to 200 mg twice daily.

Aspect 128. A method of preventing ischemic stroke in an adult patient after an acute ischemic stroke or high risk transient ischemic attack, wherein the method comprises administering to the adult patient a regimen comprising milvexian (or pharmaceutically acceptable salt or solvate thereof) in an amount of 25 mg twice daily.

Aspect 129. A method of reducing the risk of ischemic stroke in an adult patient after an acute ischemic stroke or high risk transient ischemic attack, wherein the method comprises administering to the adult patient a regimen comprising milvexian (or pharmaceutically acceptable salt or solvate thereof) in an amount of 25 mg twice daily.

Aspect 130. The method of any one of aspects 126-129, further comprising administering to the adult patient the standard of care.

Aspect 131. The method of aspect 130, wherein the standard of care comprises antiplatelet therapy.

Aspect 132. The method of aspect 131, wherein the antiplatelet therapy comprises single antiplatelet therapy.

Aspect 133. The method of aspect 132, wherein the single antiplatelet therapy is aspirin.

Aspect 134. The method of aspect 133, wherein the aspirin is administered in an amount of 75-100 mg daily.

Aspect 135. The method of aspect 134, wherein the aspirin is administered in an amount of 75 mg daily.

Aspect 136. The method of aspect 134, wherein aspirin is administered in an amount of 81 mg daily.

Aspect 137. The method of aspect 134, wherein aspirin is administered in an amount of 100 mg daily.

Aspect 138. The method of aspect 132, wherein the single antiplatelet therapy is a P2Y12 inhibitor.

Aspect 139. The method of aspect 138, wherein the P2Y12 inhibitor is clopidogrel, ticagrelor, or prasugrel.

Aspect 140. The method of aspect 139, wherein the P2Y12 inhibitor is clopidogrel.

Aspect 141. The method of aspect 140, wherein the clopidogrel is administered in an amount of 75 mg daily.

Aspect 142. The method of aspect 132, wherein the single antiplatelet therapy is ticlopidine.

Aspect 143. The method of aspect 142, wherein the ticlopidine is administered in an amount of 250-500 mg daily.

Aspect 144. The method of aspect 131, wherein the antiplatelet therapy comprises dual antiplatelet therapy.

Aspect 145. The method of aspect 144, wherein the dual antiplatelet therapy is aspirin and a P2Y12 inhibitor.

Aspect 146. The method of aspect 145, wherein the aspirin is administered in an amount of 75-100 mg daily.

Aspect 147. The method of aspect 146, wherein the aspirin is administered in an amount of 75 mg daily.

Aspect 148. The method of aspect 146, wherein aspirin is administered in an amount of 81 mg daily.

Aspect 149. The method of aspect 146, wherein aspirin is administered in an amount of 100 mg daily.

Aspect 150. The method of any one of aspects 145 to 149, wherein the P2Y12 inhibitor is clopidogrel, ticagrelor, or prasugrel.

Aspect 151. The method of aspect 150, wherein the P2Y12 inhibitor is clopidogrel.

Aspect 152. The method of aspect 151, wherein the clopidogrel is administered in an amount of 75 mg daily.

Aspect 153. The method of aspect 131, wherein the antiplatelet therapy comprises Aggrenox, i.e., a combination of aspirin and dipyridamole (persantine).

Aspect 154. The method of any one of aspects 126, 127, or 130-153, wherein the milvexian is administered in an amount of 25 mg once daily.

Aspect 155. The method of any one of aspects 126-153, wherein the milvexian is administered in an amount of 25 mg twice daily.

Aspect 156. The method of any one of aspects 126, 127, or 130-153 wherein the milvexian is administered in an amount of 50 mg twice daily.

Aspect 157. The method of any one of aspects 126, 127, or 130-153, wherein the milvexian is administered in an amount of 100 mg twice daily.

Aspect 158. The method of any one of aspects 126, 127, or 130-153, wherein the milvexian is administered in an amount of 200 mg twice daily.

Aspect 159. The method of any one of aspects 126, 127, or 130-153, wherein the milvexian is administered in an amount of less than 200 mg twice daily.

Aspect 160. The method of any one of aspects 126-159, wherein the risk of ischemic stroke is reduced compared to administering placebo with the standard of care.

Aspect 161. The method of any one of aspects 126-160, wherein the risk of ischemic stroke is reduced compared to administering placebo with antiplatelet therapy.

Aspect 162. The method of aspect 160 or aspect 161, wherein the relative risk is about 0.9-0.6.

Aspect 163. The method of aspect 160 or aspect 161, wherein the relative risk reduction is about 10-40%.

Aspect 164. The method of any one of aspects 126-163, wherein the risk of ischemic stroke decreases with the amount of milvexian administered in a dose-dependent manner.

Aspect 165. The method of any one of aspects 126-164, wherein the relative risk of bleeding according to the Bleeding Academic Research Consortium (BARC) Type 3 and 5 criteria is no greater than 3, compared to placebo with the standard of care.

Aspect 166. The method of any one of aspects 126-165, wherein the relative risk of bleeding according to the Bleeding Academic Research Consortium (BARC) Type 3 and 5 criteria is no greater than 3, compared to placebo with antiplatelet therapy.

Aspect 167. The method of any one of aspects 126-166, wherein the risk of serious bleeding according to the Bleeding Academic Research Consortium (BARC) Type 3 and 5 criteria is independent of the amount of milvexian administered.

Aspect 168. The method of any one of aspects 126-167, wherein the relative risk of bleeding according to the Bleeding Academic Research Consortium (BARC) Type 2 criteria is no greater than 2.6, compared to placebo with the standard of care.

Aspect 169. The method of any one of aspects 126-167, wherein the relative risk of bleeding according to the Bleeding Academic Research Consortium (BARC) Type 2 criteria is no greater than 2.6, compared to placebo with antiplatelet therapy.

Aspect 170. The method of any one of aspects 126-169, wherein the risk of bleeding according to the Bleeding Academic Research Consortium (BARC) Type 2 criteria is independent of the amount of milvexian administered.

Aspect 171. The method of any one of aspects 126-170, wherein the relative risk of bleeding according to the ISTH criteria (major bleeding or clinically-relevant non-major bleeding (CRNM)) is no greater than 3, compared to placebo with the standard of care.

Aspect 172. The method of any one of aspects 126-171, wherein the relative risk of bleeding according to the ISTH criteria (major bleeding or clinically-relevant non-major bleeding (CRNM)) is no greater than 3, compared to placebo with antiplatelet therapy.

Aspect 173. A pharmaceutical product that is administered twice daily to a patient who has or has had an ischemic stroke or a transient ischemic attack.

Aspect 174. The pharmaceutical product of aspect 173, wherein the amount of milvexian in each administration is in a range from about 25 mg to about 100 mg.

Aspect 175. The pharmaceutical product of aspect 174, wherein the amount of milvexian in each administration is about 25 mg.

Aspect 176. The pharmaceutical product of any one of the aspects 173-175, wherein the pharmaceutical product causes an about 30% relative risk reduction (RRR) in the occurrence of clinical ischemic stroke events in the patient without increased bleeding versus the placebo group.

Aspect 177. The pharmaceutical product of any one of the aspects 173-176, wherein the clinical benefit of reducing the incidence rates of the clinical ischemic stroke by the pharmaceutical product is maintained throughout the treatment period of 90 days.

Aspect 178. The method of any one of aspects 1-172, wherein administration of the regimen reduces the patient's FXI clotting activity by about 7% to about 20% relative to baseline.

Aspect 179. The method of any one of aspects 1-172 or 178, wherein administration of the regimen results is a prolongation of activated partial thromboplastin time (aPTT) ranging from about 27% to about 64% relative to baseline.

Aspect 180. The method of any one of aspects 1-172 or 178-179, where the administration does not result in a statistically significant increase in major bleeding complications.

Aspect 181. The method of any one of aspects 1-172 or 178-180, wherein the milvexian (or pharmaceutically acceptable salt or solvate thereof) is administered in a pharmaceutical composition that is a solid oral pharmaceutical composition.

Aspect 182. The method of aspect 181, wherein the solid oral pharmaceutical composition is a tablet.

Aspect 183. The method of aspect 182, wherein the tablet is an immediate release tablet.

Aspect 184. The method of aspect 182 or aspect 183, wherein the tablet has a disintegration time in water of less than 20 seconds.

Aspect 185. The method of any one of aspects 1-172 or 178-184, wherein the administration results in a milvexian plasma half-life ranging from about 13 hours to about 16 hours.

Aspect 186. The method of any one of aspects 1-172 or 178-185, wherein the administration results in milvexian plasma concentration reaching steady-state at about 3 days to 6 days.

Aspect 187. The method of any one of aspects 1-172 or 178-186, wherein the milvexian (or pharmaceutically acceptable salt or solvate thereof) is administered without regard to the timing of food intake.

Aspect 188. The method of any one aspects 1-172 or 178-187, wherein the human patient is unable to swallow a tablet dosage form.

Aspect 189. The method of any one of aspects 182-188, wherein the tablet is dispersed in an aqueous medium to form an aqueous dispersion.

Aspect 190. The method of aspect 189, wherein the aqueous medium is water, saline, phosphate buffer, vegetable juice, or fruit juice, including, for example, apple sauce.

Aspect 191. The method of aspect 189 or aspect 190, wherein the aqueous dispersion is administered to the human patient via a nasogastric tube or spoon.

Aspect 192. The method of any one of aspects 182-191, wherein the milvexian is administered orally as an aqueous dispersion by dispersing the oral tablet in an aqueous medium in less than 1 minute.

Aspect 193. A method of preventing, or reducing the risk of, ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves at least one of the following efficacy outcomes: (i) a relative risk for ischemic stroke of about 0.6 to about 0.9 relative to placebo dosing; (ii) a numerical reduction of the absolute risk of ischemic stroke relative to a placebo dosing; (iii) numerically reduces the Kaplan-Meier rate for ischemic stroke compared to a placebo dosing; (iv) a numerical reduction in the percentage of patients with ischemic relative to a placebo dosing; or (v) a relative risk reduction for ischemic stroke of about 10% to about 40% relative to placebo dosing.

Aspect 194. A method of preventing, or reducing the risk of, ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), preferably wherein the method achieves a relative risk for ischemic stroke of about 0.6 to about 0.9 relative to placebo dosing.

Aspect 195. A method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), preferably wherein the method achieves a relative risk reduction for ischemic stroke of about 10% to about 40% relative to placebo dosing.

Aspect 196. The method of Aspect 195, wherein the method achieves a relative risk reduction for ischemic stroke of about 28% to about 32% relative to placebo dosing.

Aspect 197. The method of Aspect 195, wherein the method achieves a relative risk reduction for ischemic stroke of about 30% relative to placebo dosing.

Aspect 198. The method of any one of Aspects 193 to 196, wherein the method further achieves at least one of the following safety outcomes: (i) no fatal bleeding events comparable to placebo dosing; (ii) no intracranial hemorrhage events comparable to placebo dosing; or (iii) an incidence rate of major bleeding (BARC Type 3 and 5) comparable to placebo dosing.

Aspect 199. The method of any one of aspects 193 to 198, wherein administration of the regimen causes a reduction of FXI clotting activity in the patient of about 7% to about 20% relative to baseline.

Aspect 200. The method of any one of Aspects 193 to 198, wherein administration of the regimen causes a prolongation of activated partial thromboplastin time (aPTT) in the patient ranging from about 27% to about 64% relative to baseline.

Aspect 201. The method of any one of Aspects 193 to 196, wherein the milvexian (or pharmaceutically acceptable salt or solvate thereof) is administered in a pharmaceutical composition that is a solid oral pharmaceutical composition.

Aspect 202. The method of Aspect 201, wherein the solid oral pharmaceutical composition is a tablet.

Aspect 203. The method of Aspect 202, wherein the tablet is an immediate release tablet.

Aspect 204. The method of Aspect 202 or Aspect 203, wherein the tablet has a disintegration time in water of less than 20 seconds.

Aspect 205. The method of any one of Aspects 193 to 198, or Aspects 201 to 204, wherein the administration results in a milvexian plasma half-life ranging from about 13 hours to about 16 hours during repeat dosing.

Aspect 206. The method of any one of aspects 193 to 198, or Aspects 201 to 205, wherein the administration results in milvexian plasma concentration reaching steady-state at about 3 days to 6 days.

Aspect 207. The method of any one of aspects 193 to 196, or Aspects 201 to 205, wherein the milvexian (or pharmaceutically acceptable salt or solvate thereof) is administered without regard to the timing of food intake.

Aspect 208. The method of any one aspects 193 to 198, or Aspects 201 to 206, wherein the patient is unable to swallow a tablet dosage form.

Aspect 209. The method of any one of Aspects 193 to 198, wherein the tablet is dispersed in an aqueous medium to form an aqueous dispersion.

Aspect 210. The method of Aspect 209, wherein the aqueous medium is water, saline, phosphate buffer, vegetable juice, or fruit juice, including, for example, apple sauce.

Aspect 211. The method of Aspect 209 or Aspect 210, wherein the aqueous dispersion is administered to the human patient via a nasogastric tube or spoon.

Aspect 212. The method of any one of aspects 209 to 211, wherein the milvexian is administered orally as an aqueous dispersion by dispersing the oral tablet in an aqueous medium in less than 60 seconds.

Aspect 213. Milvexian for use in a method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves at least one of the following efficacy outcomes: (i) a relative risk for ischemic stroke of about 0.6 to about 0.9 relative to placebo dosing; (ii) a numerical reduction of the absolute risk of ischemic stroke relative to a placebo dosing; (iii) numerically reduces the Kaplan-Meier rate for ischemic stroke compared to a placebo dosing; (iv) a numerical reduction in the percentage of patients with ischemic relative to a placebo dosing; or (v) a relative risk reduction for ischemic stroke of about 10% to about 40% relative to placebo dosing.

Aspect 214. Milvexian for use in a method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), preferably wherein the method achieves a relative risk for ischemic stroke of about 0.6 to about 0.9 relative to placebo dosing.

Aspect 215. Milvexian for use in a method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), preferably wherein the method achieves a relative risk reduction for ischemic stroke of about 10% to about 40% relative to placebo dosing.

Aspect 216. The milvexian for use according to Aspect 215, wherein the method achieves a relative risk reduction for ischemic stroke of about 10% to about 40% relative to placebo dosing.

Aspect 217. The milvexian for use according to Aspect 215, wherein the method achieves a relative risk reduction for ischemic stroke of about 28% to about 32% relative to placebo dosing.

Aspect 218. The milvexian for use according to any one of Aspects 213-217, wherein the method further achieves at least one of the following safety outcomes: (i) no fatal bleeding events comparable to placebo dosing; (ii) no intracranial hemorrhage events comparable to placebo dosing; or (iii) an incidence rate of major bleeding (BARC Type 3 and 5) comparable to placebo dosing.

Aspect 219. The milvexian for use according to any one of Aspects 213-217, wherein administration of the regimen causes a reduction of FXI clotting activity in the patient of about 7% to about 20% relative to baseline.

Aspect 220. The milvexian for use according to any one of Aspects 213-217, wherein administration of the regimen causes a prolongation of activated partial thromboplastin time (aPTT) in the patient ranging from about 27% to about 64% relative to baseline.

Aspect 221. The milvexian for use according to any one of Aspects 213-217, wherein the milvexian (or pharmaceutically acceptable salt or solvate thereof) is administered in a pharmaceutical composition that is a solid oral pharmaceutical composition.

Aspect 222. The milvexian for use according to Aspect 221, wherein the solid oral pharmaceutical composition is a tablet.

Aspect 223. The method of Aspect 202, wherein the tablet is an immediate release tablet.

Aspect 224. The method of Aspect 202 or Aspect 203, wherein the tablet has a disintegration time in water of less than 20 seconds.

Aspect 225. The method of any one of Aspects 213-217, or Aspects 221 to 224, wherein the administration results in a milvexian plasma half-life ranging from about 13 hours to about 16 hours during repeat dosing.

Aspect 226. The method of any one of Aspects 213-217, or Aspects 221 to 225, wherein the administration results in milvexian plasma concentration reaching steady-state at about 3 days to 6 days.

Aspect 227. The method of any one of Aspects 213-217, or Aspects 221 to 226, wherein the milvexian (or pharmaceutically acceptable salt or solvate thereof) is administered without regard to the timing of food intake.

Aspect 228. The method of any one Aspects 213-217, or Aspects 221 to 227, wherein the patient is unable to swallow a tablet dosage form.

Aspect 229. The method of any one of Aspects 213-217, wherein the tablet is dispersed in an aqueous medium to form an aqueous dispersion.

Aspect 230. The method of Aspect 229, wherein the aqueous medium is water, saline, phosphate buffer, vegetable juice, or fruit juice, including, for example, apple sauce.

Aspect 231. The method of Aspect 229 or Aspect 230, wherein the aqueous dispersion is administered to the human patient via a nasogastric tube or spoon.

Aspect 232. The method of any one of aspects 229 to 231, wherein the milvexian is administered orally as an aqueous dispersion by dispersing the oral tablet in an aqueous medium in less than 60 seconds.

Aspect 233. The method of any one of aspects 193 to 232, wherein the antiplatelet therapy is single antiplatelet therapy.

Aspect 234. The method of aspect 233, wherein the single antiplatelet therapy is aspirin.

Aspect 235. The method of aspect 234, wherein the aspirin is administered in an amount of 75-100 mg daily.

Aspect 236. The method of aspect 235, wherein the aspirin is administered in an amount of 75 mg daily.

Aspect 237. The method of aspect 235, wherein aspirin is administered in an amount of 81 mg daily.

Aspect 238. The method of aspect 235, wherein aspirin is administered in an amount of 100 mg daily.

Aspect 239. The method of any one of aspects 193 to 232, wherein the single antiplatelet therapy is a P2Y12 inhibitor.

Aspect 240. The method of aspect 239, wherein the P2Y12 inhibitor is clopidogrel, ticagrelor, or prasugrel.

Aspect 241. The method of aspect 240, wherein the P2Y12 inhibitor is clopidogrel.

Aspect 242. The method of aspect 242, wherein the clopidogrel is administered in an amount of 75 mg daily.

Aspect 243. The method of any one of aspects 193 to 232, wherein the single antiplatelet therapy is ticlopidine.

Aspect 244. The method of aspect 243, wherein the ticlopidine is administered in an amount of 250-500 mg daily.

Aspect 245. The method of any one of aspects 193 to 232, wherein the antiplatelet therapy is dual antiplatelet therapy.

Aspect 246. The method of aspect 245, wherein the dual antiplatelet therapy is aspirin and a P2Y12 inhibitor.

Aspect 247. The method of aspect 246, wherein the aspirin is administered in an amount of 75-100 mg daily.

Aspect 248. The method of aspect 247, wherein the aspirin is administered in an amount of 75 mg daily.

Aspect 249. The method of aspect 247, wherein aspirin is administered in an amount of 81 mg daily.

Aspect 250. The method of aspect 247, wherein aspirin is administered in an amount of 100 mg daily.

Aspect 251. The method of any one of aspects 245 to 250, wherein the P2Y12 inhibitor is clopidogrel, ticagrelor, or prasugrel.

Aspect 252. The method of aspect 251, wherein the P2Y12 inhibitor is clopidogrel.

Aspect 253. The method of aspect 252, wherein the clopidogrel is administered in an amount of 75 mg daily.

Aspect 254. The method of any one of aspects 193 to 232, wherein the antiplatelet therapy comprises Aggrenox, i.e., a combination of aspirin and dipyridamole (persantine).

Aspect 255. The method of any one of aspects 193 to 254, wherein the pharmaceutical composition is a tablet having the composition and/or properties shown in Table A.

Aspect 256. The method of Aspect 255, wherein the tablet comprises spray-dried amorphous solid dispersion (SDP).

Aspect 257. The method of Aspect 255, wherein the tablet comprises about 22.0% w/w of spray-dried amorphous solid dispersion (SDP) by the total weight of the tablet.

Aspect 258. The method of Aspect 255, wherein the tablet comprises about 43.0% w/w of silicified microcrystalline cellulose by the total weight of the tablet.

Aspect 259. The method of Aspect 255, wherein the tablet comprises about 22.0% w/w of SDP and about 43.0% w/w of silicified microcrystalline cellulose by the total weight of the tablet.

Aspect 260. The method of any one of Aspects 255 to 259, wherein the SDP consists essentially of the milvexian free form and hypromellose acetate succinate MG grade (HPMCAS-MG) in a weight ratio of 3:1 (milvexian: HPMCAS-MG).

Aspect 261. The method of Aspect 255, wherein the tablet consists essentially of 25 mg of milveixian, HPMCAS-MG, silicified microcrystalline cellulose, Lactose monohydrate, croscarmellose sodium, and magnesium stearate.

Aspect 262. The method of Aspect 261, wherein the tablet further has a film coating comprises polyvinyl alcohol, iron oxide, macrogol (PEG) polyvinyl alcohol grafted copolymer, and talc.

Aspect 263. A method of preventing, or reducing the risk of, ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves at least one of the following efficacy outcomes: (i) a relative risk for ischemic stroke of about 0.6 to about 0.9 relative to placebo dosing; (ii) a numerical reduction of the absolute risk of ischemic stroke relative to a placebo dosing; (iii) numerically reduces the Kaplan-Meier rate for ischemic stroke compared to a placebo dosing; (iv) a numerical reduction in the percentage of patients with ischemic relative to a placebo dosing; or (v) a relative risk reduction for ischemic stroke of about 10% to about 40% relative to placebo dosing.

Aspect 264. A method of preventing, or reducing the risk of, ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves a relative risk for ischemic stroke of about 0.6 to about 0.9 relative to placebo dosing.

Aspect 265. A method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves a relative risk reduction for ischemic stroke of about 10% to about 40% relative to placebo dosing.

Aspect 266. The method of Aspect 265, wherein the method achieves a relative risk reduction for ischemic stroke of about 28% to about 32% relative to placebo dosing.

Aspect 267. The method of Aspect 265, wherein the method achieves a relative risk reduction for ischemic stroke of about 30% relative to placebo dosing.

Aspect 268. The method of any one of Aspects 263 to 266, wherein the method further achieves at least one of the following safety outcomes: (i) no fatal bleeding events comparable to placebo dosing; (ii) no intracranial hemorrhage events comparable to placebo dosing; or (iii) an incidence rate of major bleeding (BARC Type 3 and 5) comparable to placebo dosing.

Aspect 269. The method of any one of aspects 263 to 268, wherein administration of the regimen causes a reduction of FXI clotting activity in the patient of about 7% to about 20% relative to baseline.

Aspect 270. The method of any one of Aspects 263 to 268, wherein administration of the regimen causes a prolongation of activated partial thromboplastin time (aPTT) in the patient ranging from about 27% to about 64% relative to baseline.

Aspect 271. The method of any one of Aspects 263 to 266, wherein the milvexian (or pharmaceutically acceptable salt or solvate thereof) is administered in a pharmaceutical composition that is a solid oral pharmaceutical composition.

Aspect 272. The method of Aspect 271, wherein the solid oral pharmaceutical composition is a tablet.

Aspect 273. The method of Aspect 272, wherein the tablet is an immediate release tablet.

Aspect 274. The method of Aspect 272 or Aspect 273, wherein the tablet has a disintegration time in water of less than 20 seconds.

Aspect 275. The method of any one of Aspects 263 to 268, or Aspects 271 to 274, wherein the administration results in a milvexian plasma half-life ranging from about 13 hours to about 16 hours during repeat dosing.

Aspect 276. The method of any one of aspects 263 to 268, or Aspects 271 to 275, wherein the administration results in milvexian plasma concentration reaching steady-state at about 3 days to 6 days.

Aspect 277. The method of any one of aspects 263 to 266, or Aspects 271 to 275, wherein the milvexian (or pharmaceutically acceptable salt or solvate thereof) is administered without regard to the timing of food intake.

Aspect 278. The method of any one aspects 263 to 268, or Aspects 271 to 276, wherein the patient is unable to swallow a tablet dosage form.

Aspect 279. The method of any one of Aspects 263 to 268, wherein the tablet is dispersed in an aqueous medium to form an aqueous dispersion.

Aspect 280. The method of Aspect 279, wherein the aqueous medium is water, saline, phosphate buffer, vegetable juice, or fruit juice, including, for example, apple sauce.

Aspect 281. The method of Aspect 279 or Aspect 280, wherein the aqueous dispersion is administered to the human patient via a nasogastric tube or spoon.

Aspect 282. The method of any one of aspects 279 to 281, wherein the milvexian is administered orally as an aqueous dispersion by dispersing the oral tablet in an aqueous medium in less than 60 seconds.

Aspect 283. Milvexian for use in a method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves at least one of the following efficacy outcomes: (i) a relative risk for ischemic stroke of about 0.6 to about 0.9 relative to placebo dosing; (ii) a numerical reduction of the absolute risk of ischemic stroke relative to a placebo dosing; (iii) numerically reduces the Kaplan-Meier rate for ischemic stroke compared to a placebo dosing; (iv) a numerical reduction in the percentage of patients with ischemic relative to a placebo dosing; or (v) a relative risk reduction for ischemic stroke of about 10% to about 40% relative to placebo dosing.

Aspect 284. Milvexian for use in a method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves a relative risk for ischemic stroke of about 0.6 to about 0.9 relative to placebo dosing.

Aspect 285. Milvexian for use in a method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves a relative risk reduction for ischemic stroke of about 10% to about 40% relative to placebo dosing.

Aspect 286. The milvexian for use according to Aspect 285, wherein the method achieves a relative risk reduction for ischemic stroke of about 10% to about 40% relative to placebo dosing.

Aspect 287. The milvexian for use according to Aspect 285, wherein the method achieves a relative risk reduction for ischemic stroke of about 28% to about 32% relative to placebo dosing.

Aspect 288. The milvexian for use according to any one of Aspects 283-287, wherein the method further achieves at least one of the following safety outcomes: (i) no fatal bleeding events comparable to placebo dosing; (ii) no intracranial hemorrhage events comparable to placebo dosing; or (iii) an incidence rate of major bleeding (BARC Type 3 and 5) comparable to placebo dosing.

Aspect 289. The milvexian for use according to any one of Aspects 283-287, wherein administration of the regimen causes a reduction of FXI clotting activity in the patient of about 7% to about 20% relative to baseline.

Aspect 290. The milvexian for use according to any one of Aspects 283-287, wherein administration of the regimen causes a prolongation of activated partial thromboplastin time (aPTT) in the patient ranging from about 27% to about 64% relative to baseline.

Aspect 291. The milvexian for use according to any one of Aspects 283-287, wherein the milvexian (or pharmaceutically acceptable salt or solvate thereof) is administered in a pharmaceutical composition that is a solid oral pharmaceutical composition.

Aspect 292. The milvexian for use according to Aspect 291, wherein the solid oral pharmaceutical composition is a tablet.

Aspect 293. The method of Aspect 272, wherein the tablet is an immediate release tablet.

Aspect 294. The method of Aspect 272 or Aspect 273, wherein the tablet has a disintegration time in water of less than 20 seconds.

Aspect 295. The method of any one of Aspects 283-287, or Aspects 291 to 294, wherein the administration results in a milvexian plasma half-life ranging from about 13 hours to about 16 hours during repeat dosing.

Aspect 296. The method of any one of Aspects 283-287, or Aspects 291 to 295, wherein the administration results in milvexian plasma concentration reaching steady-state at about 3 days to 6 days.

Aspect 297. The method of any one of Aspects 283-287, or Aspects 291 to 296, wherein the milvexian (or pharmaceutically acceptable salt or solvate thereof) is administered without regard to the timing of food intake.

Aspect 298. The method of any one Aspects 283-287, or Aspects 291 to 297, wherein the patient is unable to swallow a tablet dosage form.

Aspect 299. The method of any one of Aspects 283-287, wherein the tablet is dispersed in an aqueous medium to form an aqueous dispersion.

Aspect 300. The method of Aspect 299, wherein the aqueous medium is water, saline, phosphate buffer, vegetable juice, or fruit juice, including, for example, apple sauce.

Aspect 301. The method of Aspect 299 or Aspect 300, wherein the aqueous dispersion is administered to the human patient via a nasogastric tube or spoon.

Aspect 302. The method of any one of aspects 299 to 301, wherein the milvexian is administered orally as an aqueous dispersion by dispersing the oral tablet in an aqueous medium in less than 60 seconds.

Aspect 303. The method of any one of aspects 263 to 302, wherein the antiplatelet therapy is single antiplatelet therapy.

Aspect 304. The method of aspect 303, wherein the single antiplatelet therapy is aspirin.

Aspect 305. The method of aspect 304, wherein the aspirin is administered in an amount of 75-100 mg daily.

Aspect 306. The method of aspect 305, wherein the aspirin is administered in an amount of 75 mg daily.

Aspect 307. The method of aspect 305, wherein aspirin is administered in an amount of 81 mg daily.

Aspect 308. The method of aspect 305, wherein aspirin is administered in an amount of 100 mg daily.

Aspect 309. The method of any one of aspects 263 to 302, wherein the single antiplatelet therapy is a P2Y12 inhibitor.

Aspect 310. The method of aspect 309, wherein the P2Y12 inhibitor is clopidogrel, ticagrelor, or prasugrel.

Aspect 311. The method of aspect 310, wherein the P2Y12 inhibitor is clopidogrel.

Aspect 312. The method of aspect 312, wherein the clopidogrel is administered in an amount of 75 mg daily.

Aspect 313. The method of any one of aspects 263 to 302, wherein the single antiplatelet therapy is ticlopidine.

Aspect 314. The method of aspect 313, wherein the ticlopidine is administered in an amount of 250-500 mg daily.

Aspect 315. The method of any one of aspects 263 to 302, wherein the antiplatelet therapy is dual antiplatelet therapy.

Aspect 316. The method of aspect 315, wherein the dual antiplatelet therapy is aspirin and a P2Y12 inhibitor.

Aspect 317. The method of aspect 316, wherein the aspirin is administered in an amount of 75-100 mg daily.

Aspect 318. The method of aspect 317, wherein the aspirin is administered in an amount of 75 mg daily.

Aspect 319. The method of aspect 317, wherein aspirin is administered in an amount of 81 mg daily.

Aspect 320. The method of aspect 317, wherein aspirin is administered in an amount of 100 mg daily.

Aspect 321. The method of any one of aspects 315 to 320, wherein the P2Y12 inhibitor is clopidogrel, ticagrelor, or prasugrel.

Aspect 322. The method of aspect 311, wherein the P2Y12 inhibitor is clopidogrel.

Aspect 323. The method of aspect 312, wherein the clopidogrel is administered in an amount of 75 mg daily.

Aspect 324. The method of any one of aspects 263 to 302, wherein the antiplatelet therapy comprises Aggrenox, i.e., a combination of aspirin and dipyridamole (persantine).

Aspect 325. The method of any one of aspects 263 to 314, wherein the pharmaceutical composition is a tablet having the composition and/or properties shown in Table A.

Aspect 326. The method of Aspect 325, wherein the tablet comprises spray-dried amorphous solid dispersion (SDP).

Aspect 327. The method of Aspect 325, wherein the tablet comprises about 22.0% w/w of spray-dried amorphous solid dispersion (SDP) by the total weight of the tablet.

Aspect 328. The method of Aspect 325, wherein the tablet comprises about 43.0% w/w of silicified microcrystalline cellulose by the total weight of the tablet.

Aspect 329. The method of Aspect 325, wherein the tablet comprises about 22.0% w/w of SDP and about 43.0% w/w of silicified microcrystalline cellulose by the total weight of the tablet.

Aspect 330. The method of any one of Aspects 325 to 329, wherein the SDP consists essentially of the milvexian free form and hypromellose acetate succinate MG grade (HPMCAS-MG) in a weight ratio of 3:1 (milvexian: HPMCAS-MG).

Aspect 331. The method of Aspect 325, wherein the tablet consists essentially of 25 mg of milveixian, HPMCAS-MG, silicified microcrystalline cellulose, Lactose monohydrate, croscarmellose sodium, and magnesium stearate.

Aspect 332. The method of Aspect 331, wherein the tablet further has a film coating comprises polyvinyl alcohol, iron oxide, macrogol (PEG) polyvinyl alcohol grafted copolymer, and talc.

Those skilled in the art will recognize, or be able to ascertain using no more than routine experimentation, many equivalents to the embodiments described herein.

All publications, patents and patent applications mentioned in this specification are herein incorporated by reference into the specification to the same extent as if each individual publication, patent or patent application was specifically and individually indicated to be incorporated herein by reference.

### EXAMPLES

### Example 1. Antithrombotic Treatment with Factor XIa Inhibition to Optimize Management of Acute Thromboembolic Events for Secondary Stroke Prevention (AXIOMATIC-SSP)

AXIOMATIC-SSP study is an international, phase IL randomized, double-blind, placebo-controlled, dose-ranging clinical trial examining the safety and efficacy of milvexian (an oral FXIa inhibitor) for the prevention of new ischemic stroke in patients receiving asprin and clopidrogrel after an acute ischemic stroke or high-risk TIA. The main objective is to determine the dose-response relationship of milvexian in participants with acute ischemic stroke or TIA who are treated with aspirin and clopidogrel. The main secondary objective is to assess the rate of major bleeding in this population compared with placebo.

### Study Population

Key inclusion criteria for patient participation are listed in Table 1.

TABLE 1
Key Inclusion Criteria
Participant and Target Disease Characteristics

| | |
|---|---|
| Ischemic stroke: a neurological deficit | TIA: acute onset neurological deficit |
| attributable to a non-lacunar acute brain | attributable to focal ischemia of the brain by |
| infarction detected by neuroimaging (CT or | history or examination, with complete |
| MRI) and relevant to the clinical symptoms | resolution of the deficit and no brain infarction |
| AND | on neuroimaging (CT or MRI) |
| NIHSS score ≤7 at time of randomization | AND |
| AND | ABCD2 score ≥6 or motor symptoms |
| Evidence of relevant intracranial or cervical | AND |
| arterial atherosclerotic plaque, ulceration, | Evidence of relevant intracranial or cervical |
| or thrombus in a feeding artery documented by | arterial atherosclerotic plaque, ulceration, or |
| imaging (either Doppler ultrasound or CTA or | thrombus in a feeding artery documented by |
| MRA or catheter angiography) | imaging (either Doppler ultrasound or CTA or |
| AND | MRA or catheter angiography) |
| mRS ≤3 before the index event (pre-morbid) | AND |
| | mRS ≤3 before the index event (pre-morbid) |

Additional criteria
Body habitus suitable for MRI and no contraindications to the performance of an MRI
No contraindications to clopidogrel or aspirin and suitable for treatment with aspirin 100
mg per day for ≥90 days
Participants who received thrombolytic therapy or mechanical thrombectomy (without
stenting) for the treatment of the index event could be enrolled if all conditions were met:
≥24 hours elapsed between end of IV thrombolytic use/thrombectomy and first
dose of study medication
Neuroimaging (either clinical imaging or study MRI) after
IV thrombolytic therapy/post-thrombectomy excluded any hemorrhagic transformation
NIHSS ≤7 at time of randomization
Post-thrombolytic therapy/post-thrombectomy INR ≤1.5 and aPTT ≤1.4 ×
ULN prior to study treatment administration
No contraindications were identified that, in the opinion of the investigator, would
preclude the start of study medication (eg, large infarct volume, procedure-related
bleeding)
All other study criteria were met
CT, computed tomography; MRI, magnetic resonance imaging; NIHSS, National Institutes of Health Stroke Scale; CTA, computed tomography angiography; MRA, magnetic resonance angiography; mRS, modified Rankin score; TIA, transient ischemic attack; IV, intravenous; INR, international normalized ratio; aPTT, activated partial thromboplastin time; ULN, upper limit of normal.

Individuals ≥40 years of age who have had an ischemic stroke or high-risk TIA were recruited and randomized within 48 hours of the onset of symptoms. Qualifying strokes were defined as symptomatic non-lacunar acute brain infarcts visible on neuroimaging (computed tomography [CT] or magnetic resonance imaging [MRI]) and relevant to the clinical symptoms. Participants were required to have a National Institutes of Health Stroke Scale (NIHSS) score of ≤7 at the time of randomization (scores range from 0-42, with higher scores indicating greater stroke severity). Petechial hemorrhagic transformation of the index infarct and cerebral microbleeds were permitted. Qualifying TIA was defined as acute onset of neurologic deficits attributable to brain ischemia with complete resolution of deficit and no brain infarction on neuroimaging and with an ABCD2 score ≥6 (scores range from 0-7, with higher scores indicating a greater risk of stroke) or motor symptoms. See Johnston S C, et al. Validation and refinement of scores to predict very early stroke risk after transient ischaemic attack. Lancet 2007; 369(9558):283-92. All participants were required to have visible intracranial or extracranial atherosclerotic plaque of any degree, but not complete occlusion on an imaging study, and a premorbid modified Rankin score (mRS) of ≤3.

Exclusion criteria for patient participation are summarized in Table 2.

TABLE 2
Key Exclusion Criteria for Patient Participation

Predicted inability to swallow study medication
Women who are pregnant or breastfeeding
Any condition that, in the opinion of the investigator, contraindicates anticoagulant therapy
or would have an unacceptable risk of bleeding such as a large infarct volume (per
investigator discretion) or uncontrolled hypertension
Hemorrhage, intracranial tumor, arteritis, large vessel dissection, AVM, or other pathology
that could account for index symptoms must be excluded by CT or MRI interpreted locally
Symptomatic carotid stenosis or intracranial artery stenosis for which endarterectomy or
angioplasty is planned within 90 days
Any condition for which chronic anticoagulation is indicated and expected to be initiated
(eg, NVAF, DVT, PE)
Requirement for continued use of DAPT for >21 days or non-aspirin antiplatelet therapy or
anticoagulant for another medical condition (eg, prophylaxis for venous thromboembolism)
History of hemorrhage into the brain, subarachnoid hemorrhage, subdural hematoma, or
spinal cord hemorrhage except CMB and minor hemorrhagic transformation of prior infarct
manifesting as scattered petechiae (HI1 according to Heidelberg classification)
History of clinically meaningful hepatic disease and/or clinically significant abnormal liver
function
Any of the following laboratory results outside of the ranges specified below prior to study
treatment administration, confirmed by repeat:
Hemoglobin <9 g/dL
Platelet count <100,000 mm³
aPTT >1.4 × ULN
INR >1.5
AST or ALT >3 × ULN
Intracranial tumor (except meningioma, which is permitted) or aneurysm >5 mm or AVM
(except treated aneurysm without history of intracranial bleed, which is permitted)
History of ESRD with eGFR <15 mL/min/1.73 m² or requiring dialysis
Planned use of anticoagulants, including warfarin or other vitamin K antagonists, oral
thrombin and factor Xa inhibitors, bivalirudin, hirudin, argatroban, unfractionated heparin,
and LMWH, with the exception of heparin or LMWH used to maintain patency of
indwelling catheters
Anticipated concomitant chronic (>14 days) use of systemic NSAIDs; NSAID use
(including COX-2 inhibitors) prior to randomization is allowed
Use of combined P-gp and strong CYP3A4 inhibitors or combined P-gp and strong
CYP3A4 inducers in the 7 days prior to randomization, or the need for ongoing treatment
with concomitant oral or intravenous therapy with combined P-gp and strong CYP3A4
inhibitors or combined P-gp and strong CYP3A4 inducers during the study
Planned concomitant use of omeprazole or esomeprazole after randomization for the
duration of clopidogrel treatment (eg, H2 blockers [except cimetidine] and other PPIs are
allowed)
History of or any of the following findings on 12-lead ECG prior to study drug
administration: atrial fibrillation, atrial flutter, complete heart block, or Mobitz II second-
degree heart block
Known SARS-CoV-2 infection within 4 weeks prior to screening
AVM, arteriovenous malformation; CT, computed tomography; MRI, magnetic resonance imaging; NVAF, non-valvular atrial fibrillation; DVT, deep vein thrombosis; PE, pulmonary embolism; DAPT, dual antiplatelet therapy; CMB, cerebral microbleed; HI1, hemorrhagic infarction type 1; aPTT, activated partial thromboplastin time; ULN, upper limit of normal; INR, international normalized ratio; AST, aspartate aminotransferase; ALT, alanine aminotransferase; ESRD, end-stage renal disease; eGFR, estimated glomerular filtration rate; LMWH, low molecular weight heparin; NSAID, non-steroidal anti-inflammatory drug; COX-2, cyclo-oxygenase 2; P-gp, P-glycoprotein; CYP3A4, cytochrome P450 3A4; PPI, proton pump inhibitor; ECG, electrocardiogram.

Potential participants were excluded if there was evidence of large vessel dissection, intracranial tumor (other than meningioma), or arteriovenous malformation that could explain the symptoms. Also excluded were those with a history of hemorrhage in the brain or spinal cord, and those with conditions that, in the opinion of the investigator, would contradict anticoagulant therapy. A requirement for dual antiplatelet therapy beyond 21 days or planned use of anticoagulants, with the exception of heparin or low molecular weight heparin for maintaining the patency of indwelling catheters, were exclusions. Known SARS-CoV-2 infection within 4 weeks prior to screening was also an exclusion.

### Study Treatment and Visits

Following informed consent, participants were randomized to milvexian or matching placebo plus open-label uncoated aspirin 100 mg and clopidogrel (loading dose of 300-600 mg, followed by 75 mg/day). Clopidogrel was administered for 21 days, while aspirin and milvexian or matching placebo were continued until day 90 (see FIG. 2). Randomization was initially to milvexian in QD doses of 25 mg and BID doses of 25 mg, 50 mg, 100 mg, and, after review of safety and efficacy data by the independent DMC, to 200 mg BID also. Despite the delayed start of randomization to the 200-mg BID dose, the study targeted at 2:1 randomization between placebo and each milvexian dose to occur by the end of enrollment.

Milvexian dosing was started within 6 hours of the baseline scan. In instances where the baseline MRI could not be performed within 48 hours of symptom onset, it could be acquired post-randomization, but no later than 72 hours from symptom onset. Study visits occurred at 21 and 90 days, with a telephone contact at 60 days. In approximately 450 patients, central laboratory tests were also obtained at 60 days. An end-of-study MRI brain scan was obtained at 90 days, with a telephone contact at 97 days. The NIHSS, the Montreal Cognitive Assessment (MoCA), and the Digit Symbol Substitution Test (DSST) were collected at the days 1, 21, and 90 visits and following any new clinical stroke.

### Study Objectives and Endpoints

The primary efficacy endpoint was the composite of incident ischemic stroke during the treatment period or new covert brain infarction detected by the comparison of 90-day and baseline MRIs. All images were read independently by 2 blinded neuroradiologists, and disagreements were resolved by consensus. A new infarct was determined if the 90-day MRI demonstrated a new lesion on the diffusion weighted imaging (DWI) sequence or the fluid attenuated inversion recovery (FLAIR) sequence consistent with an ischemic infarct. Clinically overt strokes were determined by site investigators following standardized definitions. Briefly, to qualify as an outcome event, a clinical diagnosis of stroke required a sustained increase in the NIHSS score of ≥3 points that was not explained by other factors, such as infection, or was confirmed by imaging as a new infarct. Covert brain infarction is defined as small ischemic cerebral lesions that are detected on Day 90 MRI in the absence of a symptomatic ischemic stroke event.

The secondary endpoint of major bleeding was determined by the occurrence of types 3 and 5 bleeding, according to the Bleeding Academic Research Consortium (BARC) classification system. Roxana Mehran, et al. Standardized Bleeding Definitions for Cardiovascular Clinical Trials, Circulation. 2011; 123:2736-2747. Secondary endpoints also included the volume and number of new infarcts detected by MRI imaging and the rate of clinical bleeding of any type with milvexian compared to placebo. Bleeding was additionally evaluated utilizing International Society on Thrombosis and Haemostasis (ISTH) and PLATelet inhibition and patient Outcomes (PLATO) criteria. Central laboratory samples were used to determine the dose-response relationship to PD markers as well as the PK of milvexian.

### Statistical Considerations

Sample size calculations for the primary endpoint were performed for detecting a dose-response effect, with generalized Multiple Comparisons and Modeling (gMCP-Mod) using simulations with the Dose-Finding package in R statistical analysis software. Bornkamp B, Pinheiro J, Bretz F. R: dose-finding package, ver 0.9-16. Comprehensive R archive network. Vienna, Austra: The R Foundation; 2018; CRAN.R-project. Comprehensive R archive network. Vienna, Austra: The R Foundation. Simulations of 2500 clinical trials were performed, assuming a true incidence for placebo of 15%, a plateau-shaped dose-response relationship with maximum relative risk reduction of 32% for milvexian 100 or 200 mg BID relative to placebo, and 25 mg QD as the lowest dose added to the ordered BID dose regimens. Bretz F, Pinheiro JC, Branson M. Combining multiple comparisons and modeling techniques in dose-response studies. Biometrics 2005; 61(3):738-48; Pinheiro J, Bornkamp B, Glimm E, et al. Model-based dose finding under model uncertainty using general parametric models. Stat Med 2014; 33(10):1646-61. Candidate models included an Eₘₐₓ model, a logistic model, and an exponential model.

Using these assumptions, a total of 2100 participants allocated in a 2:1:1:1:1:1 ratio to the placebo and milvexian 25 mg QD, 25 mg BID, 50 mg BID, 100 mg BID, and 200 mg BID groups would provide approximately 80% power to demonstrate a dose-response relationship, with a 1-sided type I error of 0.049. Accounting for up to 10% of participants with missing images at day 90, a sample size of approximately 2350 patients was targeted.

All randomized participants will be included in the analysis of the primary endpoint if they have either a symptomatic ischemic stroke event by day 90 or an evaluable MRI at the day 90 visit. Because of the COVID-19 pandemic, all day 90 visit MRIs will be included in the primary analysis, regardless of timing. All other efficacy analyses will be performed on a randomized (intent-to-treat, ITT) population up to day 90.

If a dose-response relationship exists, then fitted estimates for the incidence of the primary endpoint will be calculated in each of the treatment groups using the weighted average of fitted models, with weights determined from the Akaike information criteria (AIC) for each model. The observed incidence of the primary endpoint will be summarized by treatment group using the fitted model from the gMCP-Mod procedure. The gMCP-Mod will incorporate the event proportion at each dose level, and then the logit scale will be used during the rest of the gMCP-Mod procedure.

The population for the primary efficacy endpoint analysis include all randomized participants who have (i) a primary endpoint event (a new ischemic stroke during the study period or a new covert brain infarction detected by MRI on Day 90) or (ii) an evaluable MRI image on Day 90.

The population for safety analysis includes all participants who received at least one dose of study medication. This is the primary population used in safety analysis and is analyzed according to the treatment assigned at randomization.

To assess the robustness of the primary efficacy analysis, additional sensitivity analyses will be carried out using the following approaches:
A sensitivity analysis will repeat the primary analysis, but include MRIs obtained up to day 106.
The primary analysis will be repeated using all participants randomized, counting any event-free participants without a day 90 MRI as having no event (analysis #1) or as having an event (analysis #2).
The primary analysis will be repeated excluding participants with a protocol deviation meeting predefined criteria having the potential to affect interpretation of major safety or efficacy endpoint analyses, if ≥5% of participants have such a deviation.

Key subgroup analyses will be performed for demographic variables, index event type (ischemic stroke or TIA), time from onset to first dose, baseline NIHSS score, prior ischemic stroke or TIA, hypertension, thrombolysis/thrombectomy, prior antiplatelet therapy, and prior statin therapy.

For other events, incidence within each treatment group and the associated relative risk reduction between each milvexian arm and placebo will be presented. NIHSS, mRS, MoCA, and DSST (subtest of WAIS-IV) will be summarized at baseline, on days 21 and 90, and at the time of a new stroke event.

Analysis of all safety data will be performed on participants treated with ≥1 dose of double-blind drug. The proportion of participants with major bleeding events (BARC types 3 and 5), as well as BARC-, ISTH-, and PLATO-defined criteria, will be summarized by treatment group. Additional summaries will include characteristics of cerebral microbleeds, hemorrhagic transformation of ischemic stroke, and asymptomatic intracranial bleeding. Adverse event and laboratory data, as well as PK and PD data, will be summarized.

### Results

### (i) Participants

The Phase II AXIOMATIC-SSP study randomized 2,366 participants ≥40 years of age with nonlacunar, noncardioembolic ischemic stroke (NIHSS score ≤7) or high-risk TIA within 48 hours of sign or symptom onset. The trial has been conducted at 367 sites in 27 countries. Eligible participants were randomized to receive 1 of 5 doses of double blind milvexian (25, 50, 100, 200 mg twice daily, 25 mg once daily) or placebo. A 16-fold dose range (25 mg to 400 mg total daily dose) of milvexian was compared to placebo on the background of 21 days of aspirin and clopidogrel (DAPT) followed by 69 days of aspirin monotherapy (See FIG. 2).

All participants received background treatment with open-label ASA and clopidogrel for 21 days, followed by open-label ASA from Day 22 to 90. All of the 2366 randomized subjects were included in the ITT analysis set. As shown in Table 3, in the ITT analysis set, 859 (36.3%) of participants were female, and 1884 (79.6%) participants were white. The mean (SD) of age of the participants was 69.8 (10.9). As shown in Table 6, in the ITT analysis set, Of the 2366 subjects, 1675 (74.7%) subjects received at least 1 dose of study drug, therefore in the safety analysis set. In the ITT analysis set, 2272 (96.0%) participants completed the study, and 94 (4.0%/) participants prematurely discontinued the study. In the safety analysis set, 1252 (74.7%) subjects completed the study treatment, and 423 (25.3%) participants prematurely discontinued the study treatment. Demographic and baseline characteristics were balanced among the groups. Patients were well-treated with other therapies for management of glycemia and cardiovascular risks (See Table 1 and Table 2). Baseline characteristics were balanced in the milvexian compared to placebo groups and directly comparable as shown in Table 3 and Table 5 below. The baseline characteristics of the participants are summarized in the Tables 3-6 below.

TABLE 3
Basic Characteristics

| | | | Milvexian | | | | | | | | | | | |
|---|---|---|---|---|---|---|---|---|---|---|---|---|---|---|
| | | | QD | | BID | | | | | | | | | |
| | Placebo | | 25 mg | | 25 mg | | 50 mg | | 100 mg | | 200 mg | | Combined | |
| Analysis set: | 691 | | 328 | | 318 | | 328 | | 310 | | 351 | | 1675^{a} | |
| ITT (N) | | | | | | | | | | | | | | |
| Age, years (%) | | | | | | | | | | | | | | |
| Mean (SD) | 69.1 | (10.6) | 70.9 | (10.7) | 70.1 | (11.3) | 69.3 | (10.7) | 69.7 | (10.6) | 69.5 | (11.1) | 69.8 | (10.9) |
| <65 years (%) | 208 | (30.1) | 90 | (27.4) | 97 | (30.5) | 108 | (32.9) | 92 | (29.7) | 106 | (30.2) | 30.6% | |
| >=65 and <75 | 255 | (36.9) | 110 | (33.5) | 97 | (30.5) | 109 | (33.2) | 107 | (34.5) | 121 | (34.5) | 33.3% | |
| years (%) | | | | | | | | | | | | | | |
| >=75 years (%) | 228 | (33.0) | 128 | (39.0) | 124 | (39.0) | 111 | (33.8) | 111 | (35.8) | 124 | (35.3) | 36.2% | |
| Gender (%) | 254 | (36.8) | 109 | (33.2) | 118 | (37.1) | 121 | (36.9) | 112 | (36.1) | 128 | (36.5) | 605 | (36.1) |
| Female Sex | | | | | | | | | | | | | | |
| Race (%) | | | | | | | | | | | | | | |
| White | 549 | (79.5) | 257 | (78.4) | 246 | (77.4) | 260 | (79.3) | 251 | (81.0) | 288 | (82.1) | 1335 | (79.7) |
| Black | 15 | (2.2) | 5 | (1.5) | 6 | (1.9) | 8 | (2.4) | 3 | (1.0) | 5 | (1.4) | 33 | (2.0) |
| Asian | 35 | (5.1) | 12 | (3.7) | 21 | (6.6) | 13 | (4.0) | 11 | (3.5) | 17 | (4.8) | 74 | (4.4) |
| Native | 0 | | 1 | (0.3) | 1 | (0.3) | 0 | | 0 | | 0 | | 2 | (0.1) |
| Hawaiin or | | | | | | | | | | | | | | |
| other PI | | | | | | | | | | | | | | |
| Asian Indian | 2 | (0.3) | 1 | (0.3) | 2 | (0.6) | 1 | (0.3) | 0 | | 0 | | 4 | (0.2) |
| Chinese | 0 | | 0 | | 0 | | 0 | | 1 | (0.3) | 0 | | 1 | (0.1) |
| Japanese | 84 | (12.2) | 46 | (14.0) | 40 | (12.6) | 41 | (12.5) | 39 | (12.6) | 40 | (11.4) | 206 | (12.3) |
| Malay | 1 | (0.1) | 0 | | 0 | | 0 | | 0 | | 0 | | 0 | |
| Asian Other | 0 | | 0 | | 0 | | 1 | (0.3) | 0 | | 0 | | 1 | (0.1) |
| Other | 5 | (0.7) | 6 | (1.8) | 2 | (0.6) | 4 | (1.2) | 5 | (1.6) | 1 | (0.3) | 19 | (1.1) |

^{a}The 22 subjects in the discontinued 50 QD arm and 18 subjects in the discontinued 100 QD arm are included in the combined milveixan set (total 40 subjects).
SD, standard deviation.

TABLE 4
Basic Characteristics-Index Event by Treatment Analysis Set

| | | | Milvexian | | | | | | | | | | | |
|---|---|---|---|---|---|---|---|---|---|---|---|---|---|---|
| | | | QD | | BID | | | | | | | | | |
| | Placebo | | 25 mg | | 25 mg | | 50 mg | | 100 mg | | 200 mg | | Combined | |
| Analysis set: | 691 | | 328 | | 318 | | 328 | | 310 | | 351 | | 1675^{a} | |
| ITT (N) | | | | | | | | | | | | | | |
| Qualifying event | | | | | | | | | | | | | | |
| N (%) | | | | | | | | | | | | | | |
| Ischemic Stroke | 526 | (76.1) | 238 | (72.6) | 240 | (75.5) | 251 | (76.5) | 229 | (73.9) | 278 | (79.2) | 1264 | (75.5) |
| TIA | 163 | (23.6) | 89 | (27.1) | 77 | (24.2) | 77 | (23.5) | 81 | (26.1) | 73 | (20.8) | 408 | (24.4) |
| NIHSS for stroke | | | | | | | | | | | | | | |
| Qualifying events | | | | | | | | | | | | | | |
| N (%) | | | | | | | | | | | | | | |
| 0 | 124 | (23.6) | 47 | (19.7) | 61 | (25.4) | 57 | (22.7) | 47 | (20.5) | 75 | (26.3) | 293 | (23.3) |
| 1-5 | 376 | (71.5) | 185 | (77.7) | 167 | (69.6) | 182 | (72.5) | 171 | (74.7) | 187 | (67.3) | 912 | (72.2 |
| 6-7 | 17 | (3.2) | 5 | (2.1) | 8 | (3.3) | 9 | (3.6) | 6 | (2.6) | 13 | (4.7) | 41 | (3.2) |
| >7 | 4 | (0.8) | 0 | | 0 | | 0 | | 0 | | 0 | | 0 | |
| | 5 | (1.0) | 1 | (0.4) | 4 | (1.7) | 3 | (1.2) | 3 | (1.2) | 5 | (1.8) | 18 | (1.4) |
| ABCD2 Score for TIA | | | | | | | | | | | | | | |
| Qualifying Event N (%) | | | | | | | | | | | | | | |
| >=6 | 84 | (51.5) | 45 | (50.6) | 47 | (61.0) | 39 | (50.6) | 41 | (50.6) | 40 | (54.8) | 219 | (53.7) |
| <6 with Motor | 72 | (44.2) | 39 | (43.8) | 28 | (36.4) | 35 | (45.5) | 38 | (46.9) | 31 | (42.5) | 174 | (42.6) |
| Symptoms | | | | | | | | | | | | | | |
| <6 no Motor | 0 | | 1 | (1.1) | 0 | | 0 | | 0 | | 0 | | 2 | (0.5) |
| Symptoms | | | | | | | | | | | | | | |
| Not Reported | 7 | (4.3) | 4 | (4.5) | 2 | (2.6) | 3 | (3.9) | 5 | (2.5) | 2 | (2.7) | 13 | (3.2) |
| Time From symptom | | | | | | | | | | | | | | |
| onset to 1^{st} dose N (%) | | | | | | | | | | | | | | |
| <12 hrs | 21 | (3.0) | 15 | (4.6) | 15 | (4.6) | 10 | (3.0) | 8 | (2.6) | 9 | (2.6) | 59 | (3.5) |
| 12-24 hrs | 99 | (14.3) | 47 | (14.3) | 38 | (11.9) | 43 | (13.1) | 37 | (11.9) | 44 | (12.5) | 214 | (12.8) |
| >24-48 hrs | 539 | (78) | 253 | (77.1) | 244 | (76.7) | 262 | (79.9) | 248 | (80.0) | 282 | (80.3) | 1320 | (78.8) |
| >48 hrs | 21 | (3.0) | 9 | (2.7) | 14 | (4.4) | 8 | (2.4) | 13 | (4.2) | 9 | (2.6) | 54 | (3.2) |
| Not Reported | 11 | (1.6) | 4 | (1.2) | 7 | (2.2) | 5 | (1.5) | 4 | (1.3) | 7 | (2.0) | 28 | (1.7) |

^{a}The 22 subjects in the discontinued 50 QD arm and 18 subjects in the discontinued 100 QD arm are included in the combined milveixan set (total 40 subjects).

TABLE 5
Baseline Conditions Summary by Treatment ITT Analysis Set

| | | | Milvexian | | | | | | | | | | | |
|---|---|---|---|---|---|---|---|---|---|---|---|---|---|---|
| | | | QD | | BID | | | | | | | | | |
| | Placebo | | 25 mg | | 25 mg | | 50 mg | | 100 mg | | 200 mg | | Combined | |
| Analysis set: | 691 | | 328 | | 318 | | 328 | | 310 | | 351 | | 1675^{a} | |
| ITT (N) | | | | | | | | | | | | | | |
| Hypertension | 549 | (79.5) | 255 | (77.7) | 240 | (75.5) | 251 | (76.5) | 242 | (78.1) | 266 | (75.8) | 1286 | (76.8) |
| (%) | | | | | | | | | | | | | | |
| Diabetes | 222 | (32.1) | 103 | (31.4) | 102 | (32.1) | 87 | (26.5) | 95 | (30.6) | 106 | (30.2) | 510 | (30.4) |
| Mellitus (%) | | | | | | | | | | | | | | |
| Hypercholest | 398 | (57.6) | 192 | (58.5) | 176 | (55.3) | 195 | (59.5) | 193 | (62.3) | 226 | (64.4) | 1009 | (60.2) |
| erolemia (%) | | | | | | | | | | | | | | |
| Smoking (%) | 374 | (54.1) | 174 | (53.0) | 149 | (46.9) | 164 | (50.0) | 166 | (53.5) | 188 | (53.6) | 862 | (51.5) |
| Median Blood | | | | | | | | | | | | | | |
| Pressure (mmHg) | | | | | | | | | | | | | | |
| Systolic | 145 | | 143 | | 146 | | 144 | | 145 | | 142 | | 144 | |
| Diastolic | 80 | | 80 | | 80 | | 80 | | 80 | | 80 | | 80 | |
| Prior Event (%) | | | | | | | | | | | | | | |
| Stroke | 91 | (13.2) | 44 | (13.4) | 52 | (16.4) | 38 | (11.6) | 51 | (16.5) | 48 | (13.7) | 240 | (14.3) |
| TIA | 60 | (8.7) | 32 | (9.8) | 31 | (9.7) | 20 | (6.1) | 30 | (9.7) | 29 | (8.3) | 145 | (8.7) |
| Prior Vascular | | | | | | | | | | | | | | |
| Disease (%) | | | | | | | | | | | | | | |
| CAD | 84 | (12.2) | 33 | (10.1) | 34 | (10.7) | 36 | (11.0) | 38 | (12.3) | 40 | (11.4) | 184 | (11.0) |
| PAD | 42 | (6.1) | 14 | (4.3) | 17 | (5.3) | 10 | (3.0) | 19 | (6.1) | 6 | (1.7) | 66 | (3.9) |

^{a}The 22 subjects in the discontinued 50 QD arm and 18 subjects in the discontinued 100 QD arm are included in the combined milveixan set (total 40 subjects).

TABLE 6
Baseline Conditions Summary by Treatment ITT Analysis Set

| | | | Milvexian | | | | | | | | | | | |
|---|---|---|---|---|---|---|---|---|---|---|---|---|---|---|
| | | | QD | | BID | | | | | | | | | |
| | Placebo | | 25 mg | | 25 mg | | 50 mg | | 100 mg | | 200 mg | | Combined | |
| Analysis set: | 691 | | 328 | | 318 | | 328 | | 310 | | 351 | | 1675^{a} | |
| ITT (N) | | | | | | | | | | | | | | |
| Completed | 529 | (76.6) | 260 | (79.3) | 242 | (76.1) | 251 | (76.5) | 230 | (74.2) | 240 | (68.4) | 1252 | (74.7) |
| Treatment | | | | | | | | | | | | | | |
| Not | 162 | (23.4) | 68 | (20.7) | 76 | (23.9) | 77 | (23.5) | 80 | (25.8) | 111 | (31.6) | 423 | (25.3) |
| Completed | | | | | | | | | | | | | | |
| Treatment | | | | | | | | | | | | | | |
| Completed | 666 | (96.4) | 319 | (97.3) | 302 | (95.0) | 316 | (96.3) | 294 | (94.8) | 336 | (95.7) | 2272 | (96.0) |
| Study | | | | | | | | | | | | | | |
| Not | 25 | (3.6) | 9 | (2.7) | 16 | (5.0) | 12 | (3.7) | 16 | (5.2) | 15 | (4.3) | 94 | (4.0) |
| Completed | | | | | | | | | | | | | | |
| Study | | | | | | | | | | | | | | |
| Death | 5 | (0.7) | 3 | (0.9) | 3 | (0.9) | 3 | (0.9) | 5 | (1.6) | 4 | (1.1) | 23 | (1.0) |
| Lost to | 2 | (0.3) | 0 | | 0 | | 2 | (0.6) | 0 | | 0 | | 4 | (0.2) |
| Follow-up | | | | | | | | | | | | | | |
| Subject | 17 | (2.5) | 6 | (1.8) | 13 | (4.1) | 7 | (2.1) | 11 | (3.5) | 11 | (3.1) | 66 | (2.8) |
| withdrew | | | | | | | | | | | | | | |
| Consent | | | | | | | | | | | | | | |

| | | | | | | | | | |
|---|---|---|---|---|---|---|---|---|---|
| Other | 1 | (0.1) | 0 | 0 | 0 | 0 | 0 | 1 | (<0.1) |

^{a}The 22 subjects in the discontinued 50 QD arm and 18 subjects in the discontinued 100 QD arm are included in the combined milveixan set (total 40 subjects).

### (ii) Efficacy: Dose Responses

The primary efficacy outcome was to assess dose response for the composite of new clinical ischemic stroke during treatment and new covert brain infarction detected by MRI on Day 90 in a modified ITT population (those participants with either a clinical ischemic stroke or a Day 90 MRI). While the incidence rate of the composite outcome was numerically lower at the 50 mg and 100 mg twice daily doses, there was no apparent trend in dose-response (placebo 16.6%, 25 mg once daily 16.2%, 25 mg twice daily 18.5%, 50 mg twice daily 14.1%, 100 mg twice daily 14.7%, 200 mg twice daily 16.4%, (% is the incidence rate, n/N %). However, milvexian numerically reduced the rate of clinical ischemic stroke (excluding covert brain infarction) in the ITT population at all doses except 200 mg twice daily, with doses from 25 to 100 mg twice daily showing approximately 30% relative risk reduction (RRR) versus placebo (placebo 5.5%, 25 mg once daily 4.6%, 25 mg twice daily 3.8%, 50 mg twice daily 4.0%, 100 mg twice daily 3.5%, 200 mg twice daily 7.7%). A similar trend was observed within 21 days, the intended treatment period with DAPT.

In the AXIOMATIC-TKR study, the milvexian dose of 25 mg twice daily showed comparable efficacy to enoxaparin with a favorable safety profile. In the AXIOMATIC-SSP study the 25 mg twice daily milvexian dose was numerically better than placebo for preventing clinical ischemic strokes (HR 0.69, 95% CI 0.36-1.30) with no further improvements in efficacy observed at the higher doses.

The primary endpoint was numerically lower at the 50 mg and 100 mg BD doses but there was no trend in dose-response. Milvexian numerically reduced the incident rates of clinical ischemic stroke events (acute IS and excluding covert brain infarction) in the ITT population at all milvexian doses except 200 mg twice daily (see Table 7 below). Milvexian is effective at reducing the clinical ischemic strokes in patients after an ischemic stroke or TIA at doses from 25 mg BID to 100 mg BID and these doses demonstrated about 30% relative risk reduction (RRR) in the occurrence of ischemic stroke in the treatment groups versus the placebo groups (See FIG. 4).

TABLE 7
Summary of Clinical Ischemic Stroke and Undetermined Stroke-All Randomized Participants

| Symptomatic Stroke | Placebo | | 25 QD | | 25 BID | | 50 BID | | 100 BID | | 200 BID | |
|---|---|---|---|---|---|---|---|---|---|---|---|---|
| Events | N = 691 | | N = 328 | | N = 318 | | N = 328 | | N = 310 | | N = 351 | |
| Subjects With Event n | 38 | (5.5) | 15 | (4.6) | 12 | (3.8) | 13 | (4.0) | 11 | (3.5) | 27 | (7.7) |
| Incidence Rate (n/N %) | | | | | | | | | | | | |
| Subjects With | 38 | (5.5) | 15 | (4.6) | 12 | (3.8) | 13 | (4.0) | 11 | (3.5) | 27 | (7.7) |
| Ischemic Stroke n | | | | | | | | | | | | |
| Incidence Rate (n/N %) | | | | | | | | | | | | |
| Subjects With | 0 | (0) | 0 | (0) | 0 | (0) | 0 | (0) | 0 | (0) | 0 | (0) |
| Undetermined Stroke n | | | | | | | | | | | | |
| Incidence Rate (n/N %) | | | | | | | | | | | | |
| Subjects With | 0 | (0) | 0 | (0) | 0 | (0) | 0 | (0) | 0 | (0) | 0 | (0) |

| | | | | | | |
|---|---|---|---|---|---|---|
| Hemorragic Stroke n | | | | | | |
| Incidence Rate (n/N %) | | | | | | |
| (95% CI) (1) | (3.8, 7.2) | (2.3, 6.8) | (1.7, 5.9) | (1.9, 6.1) | (1.5, 5.6) | (4.9, 10.5) |
| Relative Risk (RR) | | 0.83 | 0.69 | 0.72 | 0.65 | 1.40 |
| 95% CI for RR(2) | | (0.46, 1.49) | (0.36, 1.30) | (0.39, 1.33) | (0.33, 1.25) | (0.87, 2.25) |

Clinical events are included up to Day 21
(1) Wald 95% CI within group
(2)95% CIs for RR are constructed using Wald Confidence Limits
Program Source: BMS_GBS\CV010\OZA75318\Biostatistics\Production\Tables\CSR\rt-ef-clinstk.sas

(iii) Efficacy: Bleeding Risk

The primary safety (bleeding) outcome was BARC Type 3 and 5 bleeding which is shown in Table 8. There were no fatal bleedings in any treatment arms. Further, there was no intracranial hemorrhage with milvexian. The incidence rate (n/N %) of bleeding endpoint was low (0.6% to 1.6%) with no fatal bleeding. The incidence rate (%) of major bleeding (BARC Type 3 and 5) for milvexian 25 mg QD and BID doses was the same as placebo (0.6%), while a moderate increase in bleeding rates was observed in milvexian arm of 50 mg BD. (See FIG. 3).

The incidence rate (n/N %) of major bleeding (BARC 3 and 5, the main safety endpoint) was low overall (placebo 0.6%, 25 mg once daily 0.6%, 25 mg twice daily 0.6%, 50 mg twice daily 1.5%, 100 mg twice daily 1.6%, 200 mg twice daily 1.5%) with no fatal bleeding. The rate of major bleeding for milvexian 25 mg once daily and twice daily doses was similar to placebo, while a moderate increase was observed in milvexian dose arms of 50 mg twice daily and above (See Table 8).

TABLE 8
Summary of Major Bleeding (Composite of BARC Type 3 and
5) without Asymptonmatic Intracranial hemorrhage (ICH)

| | Placebo | | 25 QD | | 25 BID | | 50 BID | | 100 BID | | 200 BID | |
|---|---|---|---|---|---|---|---|---|---|---|---|---|
| | N = 682 | | N = 325 | | N = 313 | | N = 325 | | N = 306 | | N = 344 | |
| Composite Event n | 4 | (0.6) | 2 | (0.6) | 2 | (0.6) | 5 | (1.5) | 5 | (1.6) | 5 | (1.5) |
| Incidence Rate (n/N %) | | | | | | | | | | | | |
| Subjects With Composite | | | | | | | | | | | | |
| of BARC Type 3 and 5 | | | | | | | | | | | | |
| Bleeding N | | | | | | | | | | | | |
| Incidence Rate (n/N %) | | | | | | | | | | | | |
| BARC Type 3 | 4 | (0.6) | 2 | (0.6) | 2 | (0.6) | 5 | (1.5) | 5 | (1.6) | 5 | (1.5) |
| BARC Type 5 | 0 | (0) | 0 | (0) | 0 | (0) | 0 | (0) | 0 | (0) | 0 | (0) |

| | | | | | | |
|---|---|---|---|---|---|---|
| (95% CI) (1) | (0.5, 1.5) | (0.1, 2.2) | (0.1, 2.2) | (0.5, 3.6) | (0.5, 3.8) | (0.5, 3.4) |
| Relative Risk (RR) vs | | 1.05 | 1.09 | 2.62 | 2.79 | 2.48 |
| placebo (2) | | | | | | |
| 95% CI for RR vs | | (0.14, 5.89) | (0.15, 6.11) | (0.64, 11.75) | (0.68, 12.47) | (0.60, 11.09) |
| placebo (2) | | | | | | |

(1) Clopper Pearson Exact CI within group
(2) 95% CIs for Risk difference and RR are constructed using Exact Confidence Limits.
Bleeding events associated with on-treatment AEs or asymptomatic ICH detected by Day 90 MRI are included.
Program Source: BMS_GBS\CV010\OZA75318\Biostatistics\Production\Tables\CSR\rt-bl-maja.sas

TABLE 9
Bleeding by BARC Type (All Treated Participants):

| | | | Milvexian | | | | | | | | | |
|---|---|---|---|---|---|---|---|---|---|---|---|---|
| | | | QD regimen | | BID regimen | | | | | | | |
| | Placebo | | 25 mg | | 25 mg | | 50 mg | | 100 mg | | 200 mg | |
| | (n = 682) | | (n = 325) | | (n = 313) | | (n = 325) | | (n = 306) | | (n = 344) | |
| All BARC Types, n | 54 | (7.9) | 35 | (10.8) | 27 | (8.6) | 40 | (12.3) | 40 | (13.1) | 35 | (10.2) |
| (%) | | | | | | | | | | | | |
| Type 1: Not | 41 | (6.0) | 26 | (8.0) | 16 | (5.1) | 28 | (8.6) | 25 | (8.2) | 22 | (6.4) |
| actionable | | | | | | | | | | | | |
| Type 2: Requiring | 9 | (1.3) | 7 | (2.2) | 9 | (2.9) | 7 | (2.2) | 10 | (3.3) | 8 | (2.3) |
| assessment/treatment | | | | | | | | | | | | |
| Type 3a: Hgb drop | 2 | (0.3) | 1 | (0.3) | 1 | (0.3) | 1 | (0.3) | 2 | (0.7) | 3 | (0.9) |
| 3-5 g/dL or | | | | | | | | | | | | |
| transfusion | | | | | | | | | | | | |

| | | | | | | | | | | | |
|---|---|---|---|---|---|---|---|---|---|---|---|
| Type 3b: Hgb drop ≥5 | 0 | 1 | (0.3) | 1 | (0.3) | 1 | (0.3) | 3 | (1.0) | 1 | (0.3) |

| | | | | | | | | | |
|---|---|---|---|---|---|---|---|---|---|
| g/dL or requiring | | | | | | | | | |
| surgical intervention | | | | | | | | | |
| Type 3c: | 2 | (0.3) | 0 | 0 | 3 | (0.9) | 0 | 1 | (0.3) |

| | | | | | | |
|---|---|---|---|---|---|---|
| Intracranial | | | | | | |
| hemorrhage | | | | | | |
| including | | | | | | |
| symptomatic HT | | | | | | |
| Type 4: CABG- | 0 | 0 | 0 | 0 | 0 | 0 |
| related | | | | | | |
| Type 5: Fatal | 0 | 0 | 0 | 0 | 0 | 0 |

Hgb, hemoglobin; HT, hemorrhagic transformation; CABG, coronary artery bypass grafting.

As Table 9 above illustrates, milvexian had a similar rate of intracranial bleeds to placebo. No fatal bleeding and no increase in severe bleeding (e.g., symptomatic intracranial hemorrhage) with milvexian versus placebo was observed, despite all patients receiving background antiplatelet therapy. The on-study symptomatic intracranial bleed events occurred in placebo (n=2; 0.3); milvexian 50 mg BID (n=3, 0.9%); and milvexian 200 mg BID (n=1, 0.3%). There were no intracranial bleed events in milvexian dose groups of 25 mg QD, 25 mg BID and 100 mg BID. There was no dose-response observed with respect to this type of bleeding. Overall, there was no fatal bleeding and no increase in ICH (BARC-3c), the most detrimental bleeding to patients. In addition, there was no increase in BARC-3 bleeding in the 25 mg QD and 25 mg BID study arms.

Early stroke recurrence after ischemic stroke or transient ischemic attack (TIA) remains a significant risk despite advances in secondary prevention. Due to bleeding concerns, there are currently no anticoagulants used when a patient suffers a non-cardioembolic ischemic stroke and these data indicate that milvexian could provide an added benefit for stroke patients in clinical practice.

The overall AE and SAE profile for milvexian appeared to be similar to placebo, with the exception of the 200 mg twice daily dose.

Milvexian represents an antithrombotic therapy with a favorable bleeding and tolerability profile, offering a treatment option for patients who are currently untreated or undertreated due to bleeding concerns.

### (iv) Efficacy: Secondary Endpoints

Event rates of the composite of new ischemic non-fatal stroke, non-fatal myocardial infarction, or all-cause death.

Location, number, and volume of new FLAIR+DWI lesions.

National Institutes of Health Stroke Scale (NIHSS), Modified Rankin Scale (mRS), Montreal Cognitive Assessment (MoCA), and Digit Symbol Substitution Test (subtest of WAIS-IV) were collected at baseline (at randomization for NIHSS), on Days 21 and 90, and at the time of a new stroke event. MoCA and DSST (subset of WAIS-IV) were read by a third-party central laboratory blinded to treatment.

Descriptive statistics and corresponding change from baseline (where applicable) assessed by the National Institutes of Health Stroke Score (NIHSS), modified Rankin Scale (mRS), Montreal Cognitive Assessment (MoCA), and Digit Symbol Substitution Test (subtest of WAIS-IV) were summarized by treatment group.

### Baseline Characteristics

Overall, most index events (75.7%) were ischemic stroke, with an NIHSS ≤5 in approximately 96% of randomized subjects.

The baseline disease characteristics were balanced among the treatment groups, with the exception to NIHSS score 6 to 7 in the 200 mg BID arm (4.7%) which was higher than in the other groups (range: 2.1% to 4.0%). The NIHSS score was only increased to 7 with the latest protocol (version 06) in order to broaden the study population. Randomization to the 200 mg BID arm did not occur until safety data in this population was collected and a subsequent DMC assessment could be performed due to safety precautions. In the same protocol revision, the NIHSS score inclusion criterion was increased from ≤5 to ≤7. Thus, more subjects with NIHSS score 6 or 7 were assigned to the highest dose group due to the staggered opening of that treatment arm.

Baseline stroke imaging evaluations (MRI) were performed in almost all (99.6%; 1668/1675) subjects randomized to milvexian and almost all placebo subjects (99.4%; 687/691). At Day 90, a study MRI was performed in 90.7% (1520/1675) of all subjects randomized to milvexian and 89.6% (619/691) placebo subjects. Overall, approximately 4.5% of Day 90 MRIs were acquired out of the specified window of 106 days with double-blind treatment being restricted to 96 days.

Baseline MRI characteristics by treatment are summarized below:

24.7% of subjects in the placebo arm vs 23.0% in the combined milvexian arms did not have a recent ischemic lesion detectable on the DWI sequence consistent with the proportion of TIA patients randomized in the study

7.9% of subjects in the placebo arm vs 9.3% in the combined milvexian arms had lacunar infarcts detected by MRI.

The median number of recent ischemic lesions at baseline was 2.0 across all study treatment arms.

The median volume of recent ischemic lesions at baseline were 1.6 mL and 1.4 mL for the placebo and all milvexian treatment arms, respectively (Table 14.2.5).

A quarter of subjects (25.0%) had chronic ischemic infarcts.

There was evidence of chronic hemorrhages in 27.4% of subjects.

A quarter of subjects (25.8%) had microbleeds (≤10 mm), and 0.9% to 2.7% of subjects had macrobleeds >10 mm.

White matter abnormalities were detected in 93.7% of across all treatment arms.

The above MRI characteristics were well balanced between placebo and milvexian treatment arms.

Composite of New Ischemic Stroke, Myocardial Infarction, and all-Cause Mortality

The rate of the composite event of new ischemic stroke, ML, and all-cause death was lower in milvexian than placebo at dose levels of 25 mg QD to 100 mg BID. See Table 10 below. The relative risk of this composite event in milvexian compared with placebo ranged from 0.78 to 0.85 over the 25 mg QD to 100 mg BID range, but there was no apparent dose-response trend.

In contrast, the milvexian 200 mg BID dose group had a numerically higher rate of this composite endpoint than placebo (respectively: 9.4% and 6.1%; relative risk: 1.55), with a higher incidence of ischemic stroke (respectively: 7.7% and 5.5%), myocardial infarction (respectively: 0.9%/and 0.3%) and all cause death (respectively: 1.1% and 0.7%).

TABLE 10
Descriptive Summary of Event Rate of Composite of New Ischemic
Stroke, MI and All Cause Death - All Randomized Subjects

| New Ischemic | | | | | | | | | | | | |
|---|---|---|---|---|---|---|---|---|---|---|---|---|
| Stroke, MI And | Placebo | | 25 QD | | 25 BID | | 50 BID | | 100 BID | | 200 BID | |
| All Cause Death | N = 691 | | N = 328 | | N = 318 | | N = 328 | | N = 310 | | N = 351 | |
| Subjects With | 42 | (6.1) | 17 | (5.2) | 15 | (4.7) | 16 | (4.9) | 16 | (5.2) | 33 | (9.4) |
| Event (%) | | | | | | | | | | | | |
| Ischemic Stroke (%) | 38 | (5.5) | 15 | (4.6) | 12 | (3.8) | 13 | (4.0) | 11 | (3.5) | 27 | (7.7) |
| Undetermined | 0 | | 0 | | 0 | | 0 | | 0 | | 0 | |
| Stroke (%) | | | | | | | | | | | | |
| Myocardial | 2 | (0.3) | 1 | (0.3) | 2 | (0.6) | 1 | (0.3) | 2 | (0.6) | 3 | (0.9) |
| Infarction (%) | | | | | | | | | | | | |
| All Cause Death (%) | 5 | (0.7) | 3 | (0.9) | 3 | (0.9) | 2 | (0.6) | 5 | (1.6) | 4 | (1.1) |

| | | | | | | |
|---|---|---|---|---|---|---|
| (95% CI) (1) | (4.3, 7.9) | (2.8, 7.6) | (2.4, 7.0) | (2.5, 7.2) | (2.7, 7.6) | (6.3, 12.5) |
| Relative Risk (RR) | | 0.85 | 0.78 | 0.80 | 0.85 | 1.55 |
| 95% CI For RR (2) | | (0.49, 1.47) | (0.44, 1.38) | (0.46, 1.41) | (0.49, 1.49) | (1.00, 2.40) |

Note:
Clinical events and deaths are included up to Day 90. The randomization of subjects to the 50 mg QD (N = 22) and 100 mg QD (N = 18) treatment arms was terminated with the implementation of revised protocol 05.
(1) Wald 95% CI within group
(2) 95% CIs for RR are constructed using Wald Confidence Limits

### Location, Number, and Volume of New FLAIR+DWI Lesions

Quantitative and vascular territory information regarding incident infarcts detected by MRI at Day 90 is summarized in Tables 11 and 12 below. At baseline, recent ischemic lesions were detected and quantified using the DWI sequence. Incident infarcts (defined as new imaging lesions compared to the baseline MRI which may be associated with a symptomatic ischemic stroke or be covert in nature) were detected at Day 90 on the FLAIR sequence and categorized as DWI positive or DWI negative based on whether they could also be detected on the DWI sequence. DWI positive lesions at Day 90 were likely associated with infarctions occurring late during the follow-up period (approximately from Day 30 to Day 90) while DWI negative lesions were more likely related to earlier infarctions (approximately occurring by Day 30). The vascular territory of incident infarcts was also assessed at Day 90. Key results are as follows:

The rate of incident infarcts detected by MRI at Day 90 was numerically lower in milvexian than placebo at dose levels of 50 BID to 200 BID with no apparent dose response.

A majority of the incident infarcts were a single lesion on the MRI, and less than 3% of subjects had multiple incident infarcts across different treatment groups without an apparent relationship to dosing.

The mean volume of incident infarcts was small (0.9 to 3.0 mL) and numerically lower in milvexian than placebo at all dose levels except in the 25 mg BID arm.

Among subjects with incident infarcts detected by MRI at Day 90, a higher proportion had DWI positive lesions (range: 8.7% to 12.6%) than DWI negative lesions (range: 3.3% to 6.0%).

The mean volume of DWI positive incident infarcts was numerically lower in milvexian than placebo at all dose levels. The mean volume of DWI negative incident infarcts was numerically lower in milvexian than placebo at dose levels of 25 QD and 50 BID to 200 BID.

A number of incident infarcts were located in a new vascular territory as the index stroke. The most common new vascular location for incident infarcts was the middle cerebral artery (MCA).

TABLE 11
Descriptive Summary of Number and Volume of Incident Infarcts
at Day 90 - All Randomized Subjects with Evaluable MRI

| | Placebo | | 25 QD | | 25 BID | | 50 BID | | 100 BID | | 200 BID | |
|---|---|---|---|---|---|---|---|---|---|---|---|---|
| | N = 618 | | N = 305 | | N = 285 | | N = 306 | | N = 275 | | N = 311 | |
| NUMBER OF | 82 | (13.3) | 40 | (13.1) | 49 | (17.2) | 36 | (11.8) | 35 | (12.7) | 35 | (11.3) |
| SUBJECTS WITH | | | | | | | | | | | | |
| INCIDENT | | | | | | | | | | | | |
| INFARCTS (NEW | | | | | | | | | | | | |
| DWI+ OR DWI− | | | | | | | | | | | | |
| LESIONS) (%) | | | | | | | | | | | | |
| NUMBER OF | | | | | | | | | | | | |
| INCIDENT | | | | | | | | | | | | |
| INFARCTS (NEW | | | | | | | | | | | | |
| DWI+ OR DWI− | | | | | | | | | | | | |
| LESIONS) (%) | | | | | | | | | | | | |
| >0 | 82 | (13.3) | 40 | (13.1) | 49 | (17.2) | 36 | (11.8) | 35 | (12.7) | 35 | (11.3) |
| 1 | 57 | (9.2) | 26 | (8.5) | 36 | (12.6) | 26 | (8.5) | 24 | (8.7) | 23 | (7.4) |
| 2 | 11 | (1.8) | 8 | (2.6) | 5 | (1.8) | 5 | (1.6) | 3 | (1.1) | 6 | (1.9) |
| 3 | 8 | (1.3) | 5 | (1.6) | 4 | (1.4) | 1 | (0.3) | 2 | (0.7) | 2 | (0.6) |
| 4 | 1 | (0.2) | 1 | (0.3) | 2 | (0.7) | 3 | (1.0) | 2 | (0.7) | 2 | (0.6) |
| 5 | 1 | (0.2) | 0 | | 0 | | 0 | | 1 | (0.4) | 2 | (0.6) |
| >5 | 4 | (0.6) | 0 | | 2 | (0.7) | 1 | (0.3) | 3 | (1.1) | 0 | |
| VOLUME OF | | | | | | | | | | | | |
| INCIDENT | | | | | | | | | | | | |
| INFARCTS (NEW | | | | | | | | | | | | |
| DWI+ OR DWI− | | | | | | | | | | | | |
| LESIONS) (ML) | | | | | | | | | | | | |
| N | 82 | | 40 | | 49 | | 36 | | 35 | | 35 | |
| MEAN | 2.3492 | | 0.8976 | | 2.9902 | | 1.2682 | | 1.4727 | | 1.2711 | |
| MEDIAN | 0.2640 | | 0.2570 | | 0.2170 | | 0.3670 | | 0.2810 | | 0.3480 | |
| MIN, MAX | 0.003, 101.927 | | 0.004, 10.978 | | 0.013, 50.785 | | 0.020, 15.289 | | 0.031, 18.382 | | 0.013, 22.438 | |
| Q1, Q3 | 0.0880, 0.9050 | | 0.1195, 0.8675 | | 0.0750, 1.1160 | | 0.1670, 0.9385 | | 0.1010, 0.5230 | | 0.1010, 0.5190 | |
| SD | 11.40906 | | 1.96342 | | 8.28993 | | 2.72452 | | 3.78795 | | 4.05035 | |
| NEW DWI+ | 59 | (9.5) | 32 | (10.5) | 36 | (12.6) | 28 | (9.2) | 26 | (9.5) | 27 | (8.7) |
| LESION (%) | | | | | | | | | | | | |
| NUMBER OF | | | | | | | | | | | | |
| DWI+ LESIONS (%) | | | | | | | | | | | | |
| >0 | 59 | (9.5) | 32 | (10.5) | 36 | (12.6) | 28 | (9.2) | 26 | (9.5) | 27 | (8.7) |
| 1 | 40 | (6.5) | 23 | (7.5) | 28 | (9.8) | 20 | (6.5) | 19 | (6.9) | 19 | (6.1) |
| 2 | 7 | (1.1) | 4 | (1.3) | 2 | (0.7) | 4 | (1.3) | 0 | | 4 | (1.3) |
| 3 | 7 | (1.1) | 4 | (1.3) | 3 | (1.1) | 0 | | 2 | (0.7) | 1 | (0.3) |
| 4 | 0 | | 1 | (0.3) | 0 | | 3 | (1.0) | 2 | (0.7) | 2 | (0.6) |
| 5 | 1 | (0.2) | 0 | | 0 | | 0 | | 1 | (0.4) | 1 | (0.3) |
| >5 | 4 | (0.6) | 0 | | 2 | (0.7) | 1 | (0.3) | 2 | (0.7) | 0 | |
| VOLUME OF | | | | | | | | | | | | |
| DWI+ LESIONS (ML) | | | | | | | | | | | | |
| N | 59 | | 32 | | 36 | | 28 | | 26 | | 27 | |
| MEAN | 2.6237 | | 0.7842 | | 2.4308 | | 1.3004 | | 1.5223 | | 1.4997 | |
| MEDIAN | 0.2120 | | 0.2110 | | 0.1120 | | 0.2900 | | 0.2660 | | 0.2020 | |
| MIN, MAX | 0.003, 101.927 | | 0.004, 10.978 | | 0.013, 21.300 | | 0.020, 15.289 | | 0.010, 18.382 | | 0.013, 22.438 | |
| Q1, Q3 | 0.0620, 0.7560 | | 0.1085, 0.7535 | | 0.0420, 1.0985 | | 0.1075, 0.7250 | | 0.0570, 0.5230 | | 0.0660, 0.5190 | |
| SD | 13.35078 | | 1.95167 | | 5.16920 | | 3.07822 | | 4.06927 | | 4.60256 | |
| NEW DWI− | 27 | (4.4) | 11 | (3.6) | 17 | (6.0) | 10 | (3.3) | 10 | (3.6) | 11 | (3.5) |
| LESION (%) | | | | | | | | | | | | |
| NUMBER OF | | | | | | | | | | | | |
| DWI− LESIONS (%) | | | | | | | | | | | | |
| >0 | 27 | (4.4) | 11 | (3.6) | 17 | (6.0) | 10 | (3.3) | 10 | (3.6) | 11 | (3.5) |
| 1 | 22 | (3.6) | 8 | (2.6) | 13 | (4.6) | 9 | (2.9) | 7 | (2.5) | 9 | (2.9) |
| 2 | 2 | (0.3) | 3 | (1.0) | 2 | (0.7) | 1 | (0.3) | 2 | (0.7) | 1 | (0.3) |
| 3 | 3 | (0.5) | 0 | | 1 | (0.4) | 0 | | 0 | | 0 | |
| 4 | 0 | | 0 | | 1 | (0.4) | 0 | | 0 | | 0 | |
| 5 | 0 | | 0 | | 0 | | 0 | | 0 | | 1 | (0.3) |
| >5 | 0 | | 0 | | 0 | | 0 | | 1 | (0.4) | 0 | |
| VOLUME OF DWI− | | | | | | | | | | | | |
| LESIONS (ML) | | | | | | | | | | | | |

| | | | | | | |
|---|---|---|---|---|---|---|
| N | 27 | 11 | 17 | 10 | 10 | 11 |
| MEAN | 1.4013 | 0.9826 | 3.4711 | 0.9243 | 1.1966 | 0.3635 |
| MEDIAN | 0.3210 | 0.5610 | 0.3910 | 0.7605 | 0.2635 | 0.2720 |
| MIN, MAX | 0.004, 12.485 | 0.035, 3.617 | 0.040, 50.785 | 0.154, 2.628 | 0.048, 9.402 | 0.048, 1.213 |
| Q1, Q3 | 0.0880, 1.1510 | 0.2240, 1.3050 | 0.2170, 0.8980 | 0.3380, 1.2570 | 0.1710, 0.4610 | 0.1050, 0.5100 |
| SD | 2.75062 | 1.22923 | 12.19826 | 0.77448 | 2.89095 | 0.34648 |

Note:
Number and volume of incident infarcts considered are only for patients having incident infarct. The randomization of subjects to the 50 mg QD (N = 22) and 100 mg QD (N = 18) treatment arms was terminated with the implementation of revised protocol 05.

TABLE 12
Descriptive Summary of Vascular Territory of Incident Infarcts at Day
90 Compared with Baseline - All Randomized Subjects with Evaluable MRI

| | Placebo | | 25 QD | | 25 BID | | 50 BID | | 100 BID | | 200 BID | |
|---|---|---|---|---|---|---|---|---|---|---|---|---|
| VASCULAR TERRITORY | N = 618 | | N = 305 | | N = 285 | | N = 306 | | N = 275 | | N = 311 | |
| PCA | 13 | (2.1) | 8 | (2.6) | 4 | (1.4) | 6 | (2.0) | 7 | (2.5) | 7 | (2.5) |
| MCA | 29 | (4.7) | 15 | (4.9) | 14 | (4.9) | 13 | (4.2) | 13 | (4.7) | 13 | (4.7) |
| ACA | 6 | (1.0) | 1 | (0.3) | 3 | (1.1) | 4 | (1.3) | 0 | | 0 | |
| VA/BA | 0 | | 0 | | 0 | | 0 | | 0 | | 0 | |
| LENTICULO-STRIATE | 11 | (1.8) | 6 | (2.0) | 6 | (2.1) | 4 | (1.3) | 4 | (1.5) | 4 | (1.5) |
| ARTERIES | | | | | | | | | | | | |

| | | | | | | | | | | |
|---|---|---|---|---|---|---|---|---|---|---|
| AChA | 2 | (0.3) | 0 | 0 | 1 | (0.3) | 1 | (0.4) | 1 | (0.4) |

| | | | | | | | | | | | |
|---|---|---|---|---|---|---|---|---|---|---|---|
| SCA | 2 | (0.3) | 2 | (0.7) | 2 | (0.7) | 0 | 1 | (0.4) | 1 | (0.4) |
| PICA | 6 | (1.0) | 1 | (0.3) | 3 | (1.1) | 0 | 3 | (1.1) | 3 | (1.1) |

| AICA | 1 | (0.2) | 0 | | 0 | | 0 | | 0 | 0 |
|---|---|---|---|---|---|---|---|---|---|---|
| BASILAR - THALAMUS | 3 | (0.5) | 1 | (0.3) | 2 | (0.7) | 1 | (0.3) | 0 | 0 |

| | | | | | | | |
|---|---|---|---|---|---|---|---|
| BASILAR - MIDBRAIN | 0 | 1 | (0.3) | 0 | 0 | 0 | 0 |

| | | | | | | | | |
|---|---|---|---|---|---|---|---|---|
| BASILAR - PONS | 3 | (0.5) | 0 | 0 | 1 | (0.3) | 0 | 0 |

| | | | | | | |
|---|---|---|---|---|---|---|
| BASILAR - MEDULLA | 0 | 0 | 0 | 0 | 0 | 0 |

Note:
The randomization of subjects to the 50 mg QD (N = 22) and 100 mg QD (N = 18) treatment arms was terminated with the implementation of revised protocol 05.

NIHSS, mRS, MoCA, and DSST

For functional outcome measures, NIHSS and mRS scores at the time of a new stroke event were lower in milvexian treated subjects compared with placebo.

For cognitive outcome measures, MoCA, and DSST scores showed an overall improvement post baseline across different treatment arms including placebo.

Descriptive summaries for mRS and NIHSS are shown in Tables 13 and 14, respectively. MoCA and DSST were also assessed. In all four measures, the mean change from baseline results for Day 21 and Day 90 assessments were similar among all analyzed milvexian dose levels (except the 50 and 100 mg QD groups, which were not of sufficient size to compare) and also in placebo. There was an overall improvement of scores for stroke severity (NIHSS) and cognitive function (DSST and MoCA) in subjects for Day 21 and Day 90 assessments across all treatment groups including placebo:

The median scores for NIHSS and mRS at the time of a new stroke event were numerically lower in the milvexian arms compared with placebo.

All subjects regardless of treatment assignment showed an overall improvement in cognitive measures over the course of the study:

Mean MoCA score of 22 points at baseline increased by an average 2 points to Day 21 and Day 90 in all groups.

Mean DSST score of 31 to 37 points across all arms at baseline increased by an average of 5 to 7 points from baseline to Day 21 and an additional 1 to 2 points until Day 90. This improvement in the DSST measure did not appear to be treatment or dose dependent.

TABLE 13
Descriptive Summary of mRS for Subjects at the Time
of First Recurrent Stroke - All Randomized Subjects

| | Visit Group | | | | | | | | |
|---|---|---|---|---|---|---|---|---|---|
| | Value | | | | | | Change from Baseline | | |
| | N | Mean | SD | Min | Median | Max | Mean | SD | Median |
| BASELINE | | | | | | | | | |
| Placebo | 688 | 0.5 | 0.87 | 0 | 0.0 | 4 | | | |
| 25 QD | 326 | 0.6 | 0.95 | 0 | 0.0 | 3 | | | |
| 25 BID | 314 | 0.6 | 0.96 | 0 | 0.0 | 3 | | | |
| 50 BID | 328 | 0.5 | 0.86 | 0 | 0.0 | 4 | | | |
| 100 BID | 309 | 0.5 | 0.89 | 0 | 0.0 | 5 | | | |
| 200 BID | 349 | 0.6 | 0.95 | 0 | 0.0 | 3 | | | |
| FIRST | | | | | | | | | |
| RECURRENT | | | | | | | | | |
| STROKE | | | | | | | | | |
| Placebo | 26 | 3.0 | 1.60 | 0 | 4.0 | 5 | 2.2 | 1.67 | 2.0 |
| 25 QD | 13 | 2.7 | 1.03 | 1 | 3.0 | 5 | 1.9 | 1.19 | 2.0 |
| 25 BID | 10 | 2.6 | 1.35 | 0 | 3.0 | 4 | 2.1 | 1.45 | 2.0 |
| 50 BID | 11 | 2.3 | 1.62 | 0 | 2.0 | 4 | 1.7 | 1.62 | 1.0 |
| 100 BID | 8 | 2.9 | 1.64 | 0 | 3.0 | 5 | 2.4 | 1.92 | 2.5 |
| 200 BID | 19 | 1.8 | 1.72 | 0 | 1.0 | 5 | 1.4 | 2.01 | 1.0 |

Note:
Recurrent stroke includes Ischemic Stroke and Undetermined Stroke. The randomization of subjects to the 50 mg QD (N = 22) and 100 mg QD (N = 18) treatment arms was terminated with the implementation of revised protocol 05.

TABLE 14
Descriptive Summary of NIHSS for Subjects at the Time
of First Recurrent Stroke - All Randomized Subjects

| | Visit Group | | | | | | | | |
|---|---|---|---|---|---|---|---|---|---|
| | Value | | | | | | Change from Baseline | | |
| | N | Mean | SD | Min | Median | Max | Mean | SD | Median |
| BASELINE | | | | | | | | | |
| Placebo | 684 | 1.6 | 1.87 | 0 | 1.0 | 18 | | | |
| 25 QD | 324 | 1.7 | 1.68 | 0 | 1.0 | 7 | | | |
| 25 BID | 311 | 1.6 | 1.73 | 0 | 1.0 | 7 | | | |
| 50 BID | 326 | 1.6 | 1.79 | 0 | 1.0 | 7 | | | |
| 100 BID | 305 | 1.8 | 1.81 | 0 | 1.0 | 7 | | | |
| 200 BID | 348 | 1.6 | 1.79 | 0 | 1.0 | 7 | | | |
| FIRST | | | | | | | | | |
| RECURRENT | | | | | | | | | |
| STROKE | | | | | | | | | |
| Placebo | 29 | 6.2 | 5.01 | 0 | 6.0 | 18 | 4.2 | 4.28 | 4.0 |
| 25 QD | 14 | 4.6 | 6.79 | 0 | 3.5 | 27 | 2.6 | 7.19 | 2.0 |
| 25 BID | 11 | 5.4 | 3.17 | 0 | 5.0 | 9 | 2.6 | 2.62 | 3.0 |
| 50 BID | 11 | 4.6 | 4.30 | 0 | 3.0 | 12 | 1.6 | 2.42 | 1.0 |
| 100 BID | 10 | 7.1 | 4.68 | 2 | 6.5 | 15 | 5.2 | 5.67 | 2.5 |
| 200 BID | 20 | 4.4 | 4.6 | 0 | 2.5 | 18 | 2.8 | 4.30 | 1.0 |

Note:
Recurrent stroke includes Ischemic Stroke and Undetermined Stroke. The randomization of subjects to the 50 mg QD (N = 22) and 100 mg QD (N = 18) treatment arms was terminated with the implementation of revised protocol 05.

### (v) Efficacy: Pharmacodynamics

One objective of the study was to assess the dose-response of milvexian on pharmacodynamic (PD) biomarkers, with an endpoint of % change from baseline in aPTT and Factor XI clotting activity during treatment, measured using descriptive statistics and corresponding percent change from baseline.

A subset of the ITT Population that includes subjects with at least one PD endpoint (total 1995 subjects) assessed following the first dose of milvexian. Percent (%) change from baseline for aPTT and Factor XI clotting activity are summarized by treatment group and nominal time points. In addition, Exposure-Response relationship (E-R) analyses was conducted to explore the relationship of milvexian exposure to the percent (%) change from baseline in aPTT and Factor XI clotting activity using data collected from all treated subjects (total 2334 subjects). Dose-dependent increases in aPTT and dose-dependent decreases in the Factor XI clotting activity were observed for the milvexian treated groups.

The summary statistics for Activated Partial Thromboplastin Time (aPTT) and percent change from baseline for the pharmacodynamic population is as in the Table 15 below:

TABLE 15
Summary Statistics for Activated Partial Thromboplastin Time (aPTT)
and percent change from baseline for the pharmacodynamic population

| Visit | | | | | | | | | | |
|---|---|---|---|---|---|---|---|---|---|---|
| Lab Test: aPTT (seconds) | | | | | | | | | | |
| Time Point | | | | | | | | | | |
| Value | | | | | | Percent Change from Baseline | | | | |
| Treatment Group | | | | | | | | | | |
| N | Mean | SE | Min | Median | Max | Mean | SE | Min | Median | Max |

BASELINE Pre-dose

| | | | | | | |
|---|---|---|---|---|---|---|
| placebo | 645 | 26.46 | 0.14 | 14.7 | 25.90 | 55.9 |
| 25 mg BID | 284 | 27.19 | 0.36 | 19.1 | 26.25 | 105.7 |
| 50 mg BID | 309 | 27.20 | 0.36 | 15.0 | 26.00 | 97.3 |

DAY 1 4 h

| | | | | | | | | | | | |
|---|---|---|---|---|---|---|---|---|---|---|---|
| placebo | 575 | 27.56 | 0.37 | 18.7 | 25.90 | 121.4 | 5.18 | 1.35 | −55.3 | 0.4 | 383.7 |
| 25 mg BID | 260 | 35.06 | 0.5 | 19.9 | 34.65 | 92.1 | 31.05 | 1.79 | −34.4 | 27.06 | 121.4 |
| 50 mg BID | 276 | 41.21 | 0.71 | 22.2 | 41.65 | 94.8 | 55.31 | 2.58 | −56.1 | 55.33 | 218.2 |

DAY 1 12 h

| | | | | | | | | | | | |
|---|---|---|---|---|---|---|---|---|---|---|---|
| placebo | 570 | 27.20 | 0.56 | 14.6 | 25.90 | 319.5 | 3.21 | 1.67 | −55.8 | −0.17 | 839.7 |
| 25 mg BID | 246 | 35.27 | 0.40 | 23.8 | 34.30 | 64.7 | 32.67 | 1.57 | −37.2 | 30.41 | 141.2 |
| 50 mg BID | 267 | 43.23 | 0.53 | 21.9 | 43.20 | 86.8 | 62.29 | 2.00 | −37.6 | 60.80 | 233.8 |

DAY 1 24 h

| | | | | | | | | | | | |
|---|---|---|---|---|---|---|---|---|---|---|---|
| placebo | 582 | 26.95 | 0.48 | 14.7 | 25.75 | 251.9 | 3.35 | 1.91 | −55.5 | −1.22 | 847.0 |
| 25 mg BID | 259 | 39.68 | 0.59 | 25.7 | 38.60 | 121.3 | 49.41 | 2.40 | −67.5 | 44.40 | 354.3 |
| 50 mg BID | 269 | 49.38 | 0.604 | 26.5 | 48.60 | 105.1 | 85.17 | 2.24 | −43.2 | 81.89 | 229.5 |

DAY 21 anytime

| | | | | | | | | | | | |
|---|---|---|---|---|---|---|---|---|---|---|---|
| placebo | 559 | 25.98 | 0.14 | 14.7 | 25.50 | 46.9 | −0.78 | 0.59 | −56.1 | −1.27 | 97.4 |
| 25 mg BID | 237 | 45.20 | 0.79 | 25.9 | 43.80 | 138.1 | 69.60 | 3.28 | −66.7 | 64.26 | 425.1 |
| 50 mg BID | 268 | 57.27 | 1.13 | 23.6 | 55.10 | 292.3 | 115.62 | 4.40 | −52.7 | 112.6 | 1003.0 |

DAY 90 anytime

| | | | | | | | | | | | |
|---|---|---|---|---|---|---|---|---|---|---|---|
| placebo | 503 | 26.77 | 0.19 | 15.2 | 26.00 | 74.0 | 2.47 | 0.77 | −54.0 | 0.83 | 184.6 |
| 25 mg BID | 226 | 42.15 | 0.73 | 23.2 | 41.85 | 125.3 | 58.30 | 3.20 | −55.5 | 54.37 | 452.0 |
| 50 mg BID | 241 | 52.00 | 0.870 | 22.1 | 52.60 | 100.2 | 97.32 | 3.60 | −32.5 | 101.18 | 332.0 |

The FXI clotting activity observed in this study was as follows:

TABLE 16
Summary Statistics for Factor XI Clotting Activity Measurements
and Percent Change from Baseline - Pharmacodynamic Population

| Visit | | | | | | | | | | |
|---|---|---|---|---|---|---|---|---|---|---|
| Lab Test: FXI Clotting Activity (%) | | | | | | | | | | |
| Time Point | | | | | | | | | | |
| Value | | | | | | Percent Change | | | | |
| Treatment Group | | | | | | | | | | |
| N | Mean | SE | Min | Median | Max | Mean | SE | Min | Median | Max |

BASELINE Pre-dose

| | | | | | | |
|---|---|---|---|---|---|---|
| placebo | 645 | 113.0 | 0.93 | 10.0 | 113.0 | 150.0 |
| 25 mg BID | 281 | 112.9 | 1.27 | 37.0 | 112.0 | 150.0 |
| 50 mg BID | 300 | 113.1 | 1.30 | 35.0 | 112.5 | 150.0 |

DAY 1 4 h

| | | | | | | | | | | | |
|---|---|---|---|---|---|---|---|---|---|---|---|
| placebo | 569 | 111.6 | 1.00 | 10.0 | 113.0 | 150.0 | 2.19 | 2.78 | −89.5 | −0.93 | 1150 |
| 25 mg BID | 254 | 103.3 | 1.43 | 10.0 | 100.0 | 150.0 | −7.21 | 1.24 | −89.5 | −8.48 | 186.5 |
| 50 mg BID | 264 | 95.5 | 1.57 | 15.0 | 95.0 | 150.0 | −16.1 | 1.22 | −85.8 | −16.7 | 162.7 |

DAY 1 12 h

| | | | | | | | | | | | |
|---|---|---|---|---|---|---|---|---|---|---|---|
| placebo | 570 | 111.2 | 0.95 | 30.0 | 111.0 | 150.0 | 4.62 | 3.39 | −80.0 | −0.86 | 1170 |
| 25 mg BID | 241 | 103.1 | 1.41 | 36.0 | 101.0 | 150.0 | −6.89 | 1.34 | −63.3 | −7.63 | 202.7 |
| 50 mg BID | 258 | 91.9 | 1.36 | 40.0 | 92.0 | 148.0 | −17.8 | 0.84 | −61.7 | −17.02 | 65.7 |

DAY 1 24 h

| | | | | | | | | | | | |
|---|---|---|---|---|---|---|---|---|---|---|---|
| placebo | 581 | 114.2 | 0.92 | 33.0 | 115.0 | 150.0 | 5.01 | 2.55 | −71.1 | 0.88 | 1190 |
| 25 mg BID | 253 | 100.9 | 1.27 | 58.0 | 98.0 | 150.0 | −9.69 | 1.13 | −38.3 | −11.30 | 159.5 |
| 50 mg BID | 262 | 81.0 | 1.54 | 10.0 | 81.0 | 150.0 | −27.79 | 1.08 | −92.6 | −27.13 | 28.6 |

DAY 21 anytime

| | | | | | | | | | | | |
|---|---|---|---|---|---|---|---|---|---|---|---|
| placebo | 554 | 117.5 | 1.43 | 10.0 | 117.0 | 711 | 8.73 | 3.21 | −92.3 | 2.27 | 1340 |
| 25 mg BID | 233 | 90.52 | 1.38 | 44.0 | 88.0 | 148.0 | −18.1 | 1.24 | −67.2 | −19.10 | 121.6 |
| 50 mg BID | 258 | 65.84 | 1.55 | 12.0 | 64.0 | 148.0 | −40.8 | 1.41 | −89.5 | −42.62 | 98.0 |

DAY 90 anytime

| | | | | | | | | | | | |
|---|---|---|---|---|---|---|---|---|---|---|---|
| placebo | 503 | 112.0 | 0.98 | 10.0 | 111.0 | 150.0 | 4.48 | 3.46 | −91.6 | −1.04 | 1320 |
| 25 mg BID | 223 | 91.05 | 1.54 | 29.0 | 89.0 | 150.0 | −17.7 | 1.90 | −72.1 | −20.16 | 305.4 |
| 50 mg BID | 228 | 71.31 | 1.88 | 10.0 | 69.5 | 148.0 | −37.2 | 1.66 | −92.2 | −38.88 | 117.6 |

Nominal time points were used for PD biomarker data analysis. For post-baseline time points, N represents subjects with a baseline and a post-baseline value for that time point. Values greater than the upper limit of quantification were set to the upper limit values for summary statistics. Baseline=Non-missing result with a collection date-time less than or equal to the date-time of the first active dose of study medication in each treatment group at time point “BASELINE−PRE DOSE”. The randomization of subjects to the 50 mg QD (N=22) and 100 mg QD (N=18) treatment arms was terminated with the implementation of revised protocol 05.

### Example 2. Bioavailability Study of 25 mg and 100 mg Film Coated DC Tablet Against 25 mg and 100 mg Comparative SDP Oral Capsule in Healthy Participants and Results

The first Phase 1 trial is an open-label, randomized, crossover study to evaluate the relative oral bioavailability, pharmacokinetics, and food effect after single dose (for Part 1, Part 3, and Part 4) or multiple-dose (for Part 2). Part 1 of this first Phase 1 study is to evaluate the relative bioavailability and food effect of a single dose of 200 mg milvexian administered as film coated DC tablet compared with 100 mg comparative SDP oral capsule under fasting and fed conditions. Part 2 of this first Phase 1 study is to characterize the pharmacokinetic (PK) of multiple twice daily administered doses for 5 days of milvexian administered as 200 mg milvexian administered as film coated DC tablet and comparative SDP oral capsules at 25 mg or 200 mg. The 100 mg and 25 mg capsule formulations (see Table 17 below) are described in WO 2020210629 of which the capsule comprises MCC and lactose anhydrous DC in a weight ratio of 1:1 binder (MCC) to filler (lactose anhydrous). The composition and physical properties for the 25 mg and 100 mg film coated DC tablets are provided in the Table A above.

Blood samples were drawn at predetermined time points following drug administration as specified in the clinical study protocols. Concentration of the samples are measured using a validated analytical method (Liquid Chromatography with Tandem Mass Spectroscopy). Individual subject pharmacokinetic parameters (e.g. Cmax, AUCₗₐₛₜ, and AUC_{∞}) are derived by non-compartmental methods using Phoenix™ WinNonlin® (version 8.1, Pharsight, A Certara™ Company, L.P., Princeton, NJ, USA) software from the time-concentration profiles.

TABLE 17
SDP Capsule Formulation Composition of 100 mg and 25 mg

| | 100 mg SDP | 25 mg SDP |
|---|---|---|
| Component | capsule | capsule |
| Intragranular | | |
| SDP of 75% | 133.3 | 33.33 |
| Milvexian:HPMCAS* | | |
| Microcrystalline cellulose | 167.7 | 41.925 |
| (MCC) | | |
| Lactose anhydrous DC | 167.7 | 41.925 |
| Weight ratio of MCC to lactose | 1:1 | 1:1 |
| anhydrous | | |
| Croscarmellose sodium | 12.5 | 3.125 |
| Colloidal silicon dioxide | 2.50 | 0.625 |
| Magnesium stearate | 1.25 | 0.3125 |
| Total Intragranular | 485 | 121 |
| Extragranular | | |
| Croscarmellose sodium | 12.5 | 3.125 |
| Magnesium stearate | 2.5 | 0.625 |
| Total | 500 | 125 |

*SDP prepared according to the composition and method described in WO 2020210629

The treatment regimens for Part 1 and Part 2 are summarized in Table 18 below.

TABLE 18
Treatment Regimen used in Part 1 and Part 2

| Treatment | Formulation | Dose | Food |
|---|---|---|---|
| A (Part 1A) | 100 mg film-coated DC* | 200 mg (2 × 100 mg) | Fasted |
| | oral tablet | | |
| C (Part 1A) | 100 mg SDP oral capsule | 200 mg (2 × 100 mg) | Fasted |
| D (Part 1B) | 100 mg film-coated DC* | 200 mg (2 × 100 mg) | Fed |
| | oral tablet | | |
| F (Part 1B) | 100 mg SDP oral capsule | 200 mg (2 × 100 mg) | Fed |
| G (Part 2A) | 100 mg film-coated DC* | 200 mg (2 × 100 mg) | BID for |
| | oral tablet | | 5 days |
| H (Part 2A) | 100 mg SDP oral capsule | 200 mg (2 × 100 mg) | BID for |
| | | | 5 days |
| I (Part 2B) | 25 mg film-coated DC* | 25 mg (1 × 25 mg) | BID for |
| | oral tablet | | 5 days |
| J (Part 2B) | 25 mg SDP oral capsule | 25 mg (1 × 25 mg) | BID for |
| | | | 5 days |

*DC: direct compression
**RC: roller compaction

The absolute Bioavailability for the 100 mg SDP capsule comparative formulation is 52% for fasted and 72% for fed conditions for a 200 mg dose.

In Part 1 of single dose administration regimen, 100 mg film-coated DC tablet shows about 9.0% to about 11% as compared to the oral SDP capsule at 200 mg dose. 100 mg film-coated DC* oral tablet has lower food effects. To achieve better patient compliance, it is preferred to have milveixan being administered with or without food. A drug formulation with small food effects provide better patient compliance.

In Part 2 of multiple dose BID administration regimen, at 2×100 mg film-coated DC tablet shows about 5-7% lower bioavailability, as compared to the 2×100 mg SDP oral capsule. The 25 mg of the film-coated DC tablet (DC tablet) shows about 11-13% lower bioavailability, as compared to the 25 mg of SDP oral capsule) (See FIGS. 8A-8D for milvexian dosing curves as a function of time after BID administration).

In a second Phase 1 trial, Part 1 was an open-label, randomized, 3-way crossover study in healthy participants to evaluate the relative oral bioavailability, pharmacokinetics, and food effect of a single oral dose of 200 mg milvexian as 2×100 mg SDP DC tablet compared to 200 mg of 2×100 mg SDP granule capsule under fasting conditions and to assess the effect of food on the bioavailability of milvexian after a single dose of 200 mg milvexian as 2×100 mg SDP DC tablet. Part 2 was an open-label, randomized, 2-way crossover Study in healthy participants to evaluate the PK and relative bioavailability of a single oral dose of 50 mg milvexian as 2×25 mg SDP tablet compared to 50 mg milvexian as 2×25 mg SDP granule capsule in healthy participants under fasting conditions.

In the pooled analysis of PK (AUC_{inf} and Cₘₐₓ) data generated under the two phase 1 trials under fed and fasting conditions for 2×100 mg and 1×25 mg SDP DC tablets and 25 mg and 100 mg SDP granule capsule formulations, the results demonstrated that the tablet exhibited smaller inter-participant PK variability in healthy participants than those of capsule

The results of clinical studies demonstrated that, for tablets with similar dissolution rates, AUC_{∞} (also known as AUC_{inf}) of the 2×100 mg film-coated DC* oral tablet coated tablet relative to the 2×100 mg granule capsule formulations, met bioequivalence criteria.

1. A method of preventing, or reducing the risk of, ischemic stroke in a human patient who has or has had an ischemic stroke or transient ischemic attack, the method comprising administering to the human patient a regimen comprising milvexian (or pharmaceutically acceptable salt or solvate thereof) in an amount of 25 mg once daily or in an amount of 12.5 mg to 200 mg twice daily; and the standard of care; wherein the regimen is effective in preventing an ischemic stroke in the human patient.

2. (canceled)

3. A method of treating a human patient who has or has had an ischemic stroke or transient ischemic attack, comprising administering to the human patient a regimen comprising milvexian (or pharmaceutically acceptable salt or solvate thereof) in an amount of 25 mg once daily, or 12.5 mg to 200 mg twice daily and the standard of care; wherein the regimen is effective in reducing the risk of ischemic stroke in the human patient and wherein the human patient does not suffer an ischemic stroke during the duration of the regimen.

4. (canceled)

5. A method of treating a patient population wherein the patients have or have had an ischemic stroke or a transient ischemic attack, comprising administering to the patients in the population for a regimen comprising milvexian (or pharmaceutically acceptable salt or solvate thereof) in an amount of 25 mg once daily or in an amount of 12.5 mg to 200 mg twice daily; and the standard of care; wherein the percentage of patients in the population who experience an ischemic stroke within 90 days of beginning the regimen is less than 10%.

6. (canceled)

7. The method of claim 1 wherein the standard of care comprises antiplatelet therapy.

8. The method of claim 7, wherein the antiplatelet therapy comprises single antiplatelet therapy.

9. The method of claim 8, wherein the single antiplatelet therapy is aspirin.

10. The method of claim 8, wherein the single antiplatelet therapy is a P2Y12 inhibitor selected from the group consisting of clopidogrel, ticagrelor, ticlopidine, cangrelor, prasugrel, clopidogrel in an amount of 75 mg daily, and ticlopidine administered in an amount of 250-500 mg daily.

11. The method of claim 7, wherein the antiplatelet therapy comprises dual antiplatelet therapy.

12. The method of claim 11, wherein the dual antiplatelet therapy is aspirin and a P2Y12 inhibitor selected from the group consisting of clopidogrel, ticagrelor, ticlopidine, cangrelor, prasugrel, and clopidogrel administered in an amount of 75 mg daily.

13. The method of claim 7, wherein the antiplatelet therapy comprises Aggrenox, i.e., a combination of aspirin and dipyridamole (persantine).

14. The method of claim 1, wherein the milvexian is administered in an amount selected from the group consisting of 25 mg once daily, 25 mg twice daily, 50 mg twice daily, 100 mg twice daily, 200 mg twice daily, and less than 200 mg twice daily.

15. The method of claim 14, wherein the milvexian is administered in an amount of 25 mg twice daily.

16. The method of claim 1, wherein the risk of ischemic stroke is reduced compared to administering the placebo with antiplatelet therapy.

17. The method of claim 16, wherein the risk of ischemic stroke when administered the regimen divided by the risk of ischemic stroke when administered placebo with the standard of care (i.e., the relative risk) is about 0.6-about 0.9.

18. The method of claim 16, wherein the relative risk reduction is about 10-about 40%.

19. The method of claim 1 wherein the risk of ischemic stroke decreases with the amount of milvexian administered in a dose-dependent manner.

20. The method of claim 1, wherein the relative risk of bleeding according to the Bleeding Academic Research Consortium (BARC) Type 3 and 5 criteria is no greater than 3, compared to placebo with antiplatelet therapy.

21. The method of claim 1, wherein the relative risk of bleeding according to the Bleeding Academic Research Consortium (BARC) Type 2 criteria is no greater than 2.6, compared to placebo with antiplatelet therapy.

22. The method of claim 1, wherein the relative risk of bleeding according to the ISTH criteria (major bleeding or clinically-relevant non-major bleeding (CRNM)) is no greater than 3, compared to placebo with antiplatelet therapy.

23. The method of claim 1, wherein the human patient does not suffer bleeding according to the Bleeding Academic Research Consortium (BARC) Type 3 and 5 criteria.

24. (canceled)

25. (canceled)

26. (canceled)

27. (canceled)

28. The method of claim 1, where the administration does not result in a statistically significant increase in major bleeding complications.

29. The method of claim 1, wherein the milvexian (or pharmaceutically acceptable salt or solvate thereof) is administered in a pharmaceutical composition that is a tablet, or an immediate release tablet.

30. The method of claim 29, wherein the tablet has a disintegration time in water of less than 20 seconds.

31. The method of claim 1, wherein the administration results in a milvexian plasma half-life ranging from about 13 hours to about 16 hours during repeat dosing.

32. The method of claim 1, wherein the administration results in milvexian plasma concentration reaching steady-state at about 3 days to 6 days.

33. The method of claim 1, wherein the milvexian (or pharmaceutically acceptable salt or solvate thereof) is administered without regard to the timing of food intake.

34. A method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves a relative risk for ischemic stroke of about 0.6 to about 0.9 relative to placebo dosing.

35. A method of reducing the risk of ischemic stroke in a human patient who has or has had an ischemic stroke or transient ischemic attack, the method comprising administering to the human patient a regimen comprising (i) a pharmaceutical composition comprising milvexian (or pharmaceutically acceptable salt or solvate thereof) in an amount of 25 mg twice daily and (ii) a single antiplatelet therapy (SAPT) or dual antiplatelet therapy (DAPT), wherein the method achieves a relative risk reduction for ischemic stroke of about 10% to about 40% relative to placebo dosing.

36. The method of claim 35, wherein the method achieves a relative risk reduction for ischemic stroke of about 28% to about 32% relative to placebo dosing.

37. The method of claim 35, wherein the method achieves a relative risk reduction for ischemic stroke of about 30%.

38. The method of claim 34, wherein the method further achieves at least one of the following safety outcomes: (i) no fatal bleeding events comparable to placebo dosing; (ii) no intracranial hemorrhage events comparable to placebo dosing; or (iii) an incidence rate of major bleeding (BARC Type 3 and 5) comparable to placebo dosing.

39. (canceled)

40. (canceled)

41. (canceled)

42. (canceled)

43. (canceled)
